(19) United States
(12) Patent Application Publication
(10) Pub. No.: US 2009/0075909 A1
(43) Pub. Date: Mar. 19, 2009

(54) Derivatives Of Pentose Monosaccharides As Anti-Inflammatory Compounds

(75) Inventors: Viswajanani Jitendra Sattigeri, Haryana (IN); Sudershan K. Arora, Pune (IN); Mohammad Salman, Princeton, NJ (US); Venkata P. Palle, Haryana (IN); Muthukaman Nagarajan, Haryana (IN); Rajkumar Shirumalla, Delhi (IN); Gurpreet Kaur Aulakh, Chandigarh (IN); Abhijit Ray, Delhi (IN)

Correspondence Address: RANBAXY INC., 600 COLLEGE ROAD EAST, SUITE 2100, PRINCETON, NJ, 08540, US

(73) Assignee: RANBAXY LABORATORIES LIMITED INC., Gurgaon, Haryana (IN)

(21) Appl. No.: 11/722,573

(22) PCT Filed: Dec. 22, 2005
(86) PCT No.: PCT/IB2005/003871

§ 371 Date: May 15, 2008

Related U.S. Application Data
(60) Provisional application No. 60/638,098, filed on Dec. 22, 2004.

(51) Int. Cl. A61K 31/7008; C07H 15/04; A61P 29/00; C07H 15/12

(52) U.S. Cl. 514/25; 536/17.9; 536/17.4

(57) ABSTRACT

The present invention relates to monosaccharide derivatives as anti-inflammatory agents. The compounds of this invention can be useful for inhibition and prevention of inflammation and associated pathologies, including inflammatory and autoimmune diseases, for example, bronchial asthma, rheumatoid arthritis, type I diabetes, multiple sclerosis, allograft rejection or psoriasis. The present invention also relates to pharmacological compositions containing these monosaccharide derivatives, as well as methods of treating bronchial asthma, chronic obstructive pulmonary disease, rheumatoid arthritis, multiple sclerosis, type I diabetes, psoriasis, allograft rejection, and other inflammatory and/or auto immune disorders.

### FIELD OF THE INVENTION

The present invention relates to monosaccharide derivatives as anti-inflammatory agents. The compounds of this invention can be useful for inhibition and prevention of inflammation and associated pathologies, including inflammatory and autoimmune diseases, for example, bronchial asthma, rheumatoid arthritis, type I diabetes, multiple sclerosis, allograft rejection psoriasis, inflammatory bowel disease, ulcerative colitis, acne, atherosclerosis, cancer, pruritis or allergic rhinitis. The present invention also relates to pharmacological compositions containing these monosaccharide derivatives, as well as methods of treating bronchial asthma, chronic obstructive pulmonary disease, rheumatoid arthritis, multiple sclerosis, type I diabetes, psoriasis, allograft rejection, inflammatory bowel disease, Ulcerative colitis, acne, atherosclerosis, cancer, pruritis, allergic rhinitis and other inflammatory and/or autoimmune disorders.

### BACKGROUND OF THE INVENTION

Inflammation is a key defense mechanism of the body that is activated as a result of tissue injury. The inflammatory process is self-containing, however, under certain pathophysiological conditions inflammatory process tends to perpetuate itself giving rise to chronic inflammatory diseases, for example, bronchial asthma, rheumatoid arthritis and other diseases.

Although the exact cellular and molecular basis of most chronic inflammatory disease remain unclear, it has become apparent that several inflammatory cells act in concert towards initiation and perpetuation of an inflammatory response by releasing a wide range of chemokine, cytokine, proteolytic enzymes and other bioactive molecules. For example, mast cells primed by lymphocytes interact with environmental allergens and release mediators, for example, histamine, prostaglandin, leukotrienes etc (Clin. Exp. Allergy, 32:1682, 2002) to initiate an early inflammatory response. This is followed by a delayed inflammatory response due to release of cytokines (IL-4, IL-5, IL-6, IL-8, IL-13, GM-CSF and TNFalpha), chemokines and proteolytic enzymes (chymase, trytase)(Chest, 112:523, 1997; Lancet, 350:59, 1997) that not only bring about tissue damage, but attract other inflammatory cells and initiate tissue fibrosis, and the cycle continues. Eosinophils infiltrate inflamed tissue following allergen-mast cell interaction in bronchial asthma and allergic rhinitis. Emerging evidence indicates that mast cells also interact with bacterial endotoxins, which leads to generation of cytokines, such as TNFalpha, and encourage neutrophil influx into the site of inflammation (Br. J. Pharmacol., 123:31, 1998; Br. J. Pharmacol., 128:700, 1999; Br. J. Pharmacol., 136:111, 2002; J. Clin. Invest., 109:1351, 2002). Involvement of mast cells in the inflammatory response of chronic obstructive pulmonary disease (New Eng. J. Med., 347:1040, 2002; Thorax, 57:649, 2002), inflammatory bowel disease (Gut, 45:Suppl II6, 1999), and rheumatoid arthritis (Science, 297, 1626, 2002), as well as pathologies with prominent neutrophilic inflammation, has been proposed.

U.S. Pat. No. 6,329,344 discloses several monosaccharide derivatives as cell adhesion inhibitors. It generally relates to a group of novel substituted pentose and hexose monosaccharide derivatives, which exhibit cell adhesion inhibitory and anti-inflammatory activities. U.S. Pat. No. 6,590,085 discloses several monosaccharide derivatives as inhibitors of cell adhesion and cell adhesion mediated pathologies, including inflammatory and autoimmune diseases. U.S. Pat. No. 5,637,570 discloses disubstituted and trisubstituted derivatives of 2,3:4,6-O-isopropylidene-α-L-xylo-2-hexylofuranosonic acid having anti-cancer, anti-inflammatory and anti-poliferative activity. U.S. Pat. No. 5,298,494 discloses derivatives of monosaccharides, which exhibit anti-proliferative and/or anti-inflammatory activity and are useful for treating mammals having inflammatory disorders and/or autoimmune disorders. U.S. Pat. No. 5,367,062 discloses derivatives of disubstituted and deoxydisubstituted α,D-lyxofuranosides, which exhibit significant anti-inflammatory and antiproliferative activity, and are useful for treating inflammatory and/or autoimmune disorders. U.S. Pat. No. 5,360,794 discloses deoxydisubstituted or dideoxy disubstituted derivatives of α-D-mannofuranoside and β-L-gulofuranosides, which exhibit anti-inflammatory and antiproliferative activity. U.S. Pat. No. 4,996,195 discloses derivatives of α,D-glucofuranose and α,D-allofuranose for treating animals and mammals with inflammatory and/or autoimmune disorders. U.S. Pat. No. 5,010,058 discloses derivatives of 1,2-O-iso-propylidene-α-D-glucofuranose for treating animals and mammals with inflammatory and/or autoimmune disorders. U.S. Application No. 2002/0173632 discloses furanose and amino furanose compounds for rheumatoid, arthritis, immunomodulatory diseases inflammatory and proliferative diseases.

U.S. Application No. 2004/0023900 discloses derivatives of monosaccharides as cell adhesion inhibitors. U.S. Application No. 2004/0029820 discloses derivatives of monosaccharides as cell adhesion inhibitors. PCT Publication No. WO 93/13117 and U.S. Pat. No. 5,360,792 disclose 5- or 6-deoxy hexose monosaccharides having a saturated nitrogen containing heterocycle as anti-proliferative and anti-inflammatory compounds. PCT Publication No. WO 94/28910 discloses 5,6-dideoxy-5-amino derivatives of idose and 6-deoxy-6-amino derivatives of glucose, which exhibit immunomodulatory, anti-inflammatory and anti-proliferative activity. PCT Publication No. WO 94/11381 discloses derivatives of pentose monosaccharides as anti-proliferative and anti-inflammatory compound.

In view of the above, there remains a need for novel monosaccharide derivatives having anti-inflammatory activity.

### SUMMARY OF THE INVENTION

Generally provided herein are compounds having a structure of Formula I,

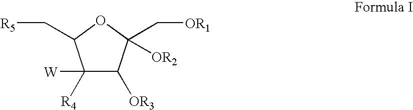

wherein
W can be hydrogen or alkyl;
R₁ can be alkyl, alkenyl, alkynyl, cycloalkyl, aryl, heterocyclyl, heteroaryl, heterocyclylalkyl, heteroarylalkyl, aralkyl or —(CH₂)ₙO(C═O)NHRₓ,
wherein n can be an integer 2-10, and
Rₓ can be alkyl, alkenyl, alkynyl, cycloalkyl, aryl, aralkyl, heteroaryl, heterocyclyl, heterocyclylalkyl or heteroarylalkyl;
   R₂ and R₃ together can form a five-membered acetal, wherein the carbon joining the two oxygen atoms can be substituted with Rₗ and Rₘ,
wherein Rₗ and Rₘ can be hydrogen, alkyl, alkenyl, alkynyl, cycloalkyl, aryl or aralkyl; Rₗ and Rₘ together can join to form a cyclic ring; or Rₗ and Rₘ together can join to form an oxo,
   wherein the ring optionally contains one or more heteroatoms selected from O, N or S, and the ring optionally can be substituted with one or more of alkyl, alkenyl, alkynyl, acyl, substituted amino, cycloalkyl, carboxy, oxo, hydroxy, alkoxy, aryloxy, halogen, aryl, aralkyl, heteroaryl, heterocyclyl, heteroarylalkyl, heterocyclylalkyl, or —C(═O)QR₇,
      wherein Q can be O or NH, and R₇ can be alkyl, alkenyl, alkynyl, aryl, aralkyl, cycloalkyl, or heteroarylalkyl; or when Q is NH, R₇ also can be heteroaryl, heterocyclyl or heterocyclylalkyl; or
         R₂ and R₃, instead of forming an acetal, optionally and independently can be lower (C₁-C₄)-alkyl, (CH₂)ₖ-aryl, —C(═R_{y})NHRₓ or acyl,
wherein k can be an integer from 1-4,
R_{y} can be O or S, and
Rₓ, can be alkyl, alkenyl, alkynyl, cycloalkyl, aryl, aralkyl, heteroaryl, heterocyclyl, heterocyclylalkyl or heteroarylalkyl; and
   R₅ can be hydrogen, alkyl, cycloalkyl, heteroaryl, heterocyclyl, —NRₚRⱼ; or OR_{z}; or when R₄ is OH, OR_{c} or H, then R₅ can be —NHC(═O)ORₛ, —NHYR_{d}, —NHC(=T)NRₜRₓ, or —(CH₂)_{w}(C═O)NRₐR_{b} (wherein w, RₐR_{b} and R_{c} same as defined earlier);
Rₚ and Rⱼ independently can be hydrogen, alkyl, cycloalkyl, aryl, heteroaryl, heterocyclyl, heteroarylalkyl, heterocyclylalkyl, or aralkyl, or Rₚ and Rⱼ together can join to form a cyclic ring, which optionally can be benzofused, containing 0-4 heteroatom selected from 0-4 heteroatoms selected from O, S or N wherein the ring can be substituted with one or more of alkyl, alkenyl, alkynyl, amino, substituted amino, cycloalkyl, carboxy, oxo, hydroxy, alkoxy, aryloxy, halogen, aryl, aralkyl, heteroaryl, heterocyclyl, heteroarylalkyl or heterocyclylalkyl;
wherein R_{z}, can be alkyl, alkenyl, alkynyl, cycloalkyl, aryl, aralkyl, heteroaryl, heterocyclyl, heteroarylalkyl, heterocyclylalkyl, acyl or —C(═O)NR_{f}R_{q},
   wherein R_{f} and R_{q} each independently can be hydrogen, alkyl, alkenyl, alkynyl, cycloalkyl, aryl, aralkyl, heterocyclyl, heteroaryl, heteroarylalkyl, heterocyclylalkyl or S(O)₂R₆; or R_{f} and R_{q} together can form a ring,
      wherein R₆ can be alkyl, alkenyl, alkynyl, cycloalkyl, aryl, aralkyl, heteroaryl, heterocyclyl, heterocyclylalkyl, heteroarylalkyl or substituted amino;
Rₛ, can be alkyl, alkenyl, alkynyl, cycloalkyl, aryl, aralkyl, heterocyclylalkyl or heteroarylalkyl,
Y can be —C(═O), —C(═S) or SO₂),
R_{d} can be alkyl, alkenyl, alkynyl, cycloalkyl, aryl, aralkyl, heteroaryl, heterocyclyl, heteroarylalkyl or heterocyclylalkyl,
T can be O, S, —N(CN), —N(NO₂), or —CH(NO₂),
Rₜ can be H, OH or Rₓ,
Rₓ, can be alkyl, alkenyl, alkynyl, cycloalkyl, aryl, aralkyl, heteroaryl, heterocyclyl, heterocyclylalkyl or heteroarylalkyl,
w can be 1-4, and
Rₐ and R_{b} each independently can be hydrogen or R_{d}, or Rₐ and R_{b}, together with the nitrogen atom carrying them, can be the N-terminus of an amino acid or di-tetrapeptide,
   wherein R_{f} and R_{q} each independently can be hydrogen, alkyl, alkenyl, alkynyl, cycloalkyl, aryl, aralkyl, heterocyclyl, heteroaryl, heteroarylalkyl, heterocyclylalkyl or S(O)₂R₆; or R_{f} and R_{q} together can form a ring,
      wherein R₆ can be allyl, alkenyl, alkynyl, cycloalkyl, aryl, aralkyl, heteroaryl, heterocyclyl, heterocyclylalkyl, heteroarylalkyl or substituted amino;
         and wherein when W is hydrogen, then R₄ can be hydrogen, OR_{c}, —NHC(═O)ORₛ, —NHYR_{d}, —NHC(=T)NRₜRₓ, or —(CH₂)_{w}(C═O)NRₐR_{b},
wherein R_{c} can be hydrogen, alkyl, cycloalkyl, aryl, heteroaryl, heterocyclyl, acyl or —C(═O)NR_{f}R_{q},
Rₛ can be alkyl, alkenyl, alkynyl, cycloalkyl, aryl, aralkyl, heterocyclylalkyl or heteroarylalkyl,
Y can be —C(═O), —C(═S) or SO₂,
R_{d} can be alkyl, alkenyl, alkynyl, cycloalkyl, aryl, aralkyl, heteroaryl, heterocyclyl, heteroarylalkyl or heterocyclylalkyl,
T can be O, S, —N(CN), —N(NO₂), or —CH(NO₂),
Rₜ can be H, OH or Rₓ,
Rₓ, can be alkyl, alkenyl, alkynyl, cycloalkyl, aryl, aralkyl, heteroaryl, heterocyclyl, heterocyclylalkyl or heteroarylalkyl,
w can be 1-4, and
Rₐ and R_{b} each independently can be hydrogen or R_{d}, or Rₐ and R_{b}, together with the nitrogen atom carrying them, can be the N-terminus of an amino acid or di-tetrapeptide,
   wherein R_{f} and R_{q} each independently can be hydrogen, alkyl, alkenyl, alkynyl, cycloalkyl, aryl, aralkyl, heterocyclyl, heteroaryl, heteroarylalkyl, heterocyclylalkyl or S(O)₂R₆; or R_{f} and R_{q} together can form a ring,
      wherein R₆ can be alkyl, alkenyl, alkynyl, cycloalkyl, aryl, aralkyl, heteroaryl, heterocyclyl, heterocyclylalkyl, heteroarylalkyl or substituted amino; or
         when W is alkyl, then R₄ can be —OR_{z},
wherein R_{z}, can be alkyl, alkenyl, alkynyl, cycloalkyl, aryl, aralkyl, heteroaryl, heterocyclyl, heteroarylalkyl, heterocyclylalkyl, acyl or —C(═O)NR_{f}R_{q},
   wherein R_{f} and R_{q} each independently can be hydrogen, alkyl, alkenyl, alkynyl, cycloalkyl, aryl, aralkyl, heterocyclyl, heteroaryl, heteroarylalkyl, heterocyclylalkyl or S(O)₂R₆; or R_{f} and R_{q} together can form a ring,
      wherein R₆ can be alkyl, alkenyl, alkynyl, cycloalkyl, aryl, aralkyl, heteroaryl, heterocyclyl, heterocyclylalkyl, heteroarylalkyl or substituted amino.

In one embodiment, Rₗ and Rₘ can join to form a cyclic ring and the cyclic ring optionally can contain one or more heteroatoms selected from O, N or S. In another embodiment, Rₗ and Rₘ can join to form a cyclic ring and the cyclic ring optionally can be substituted with one or more of alkyl, alkenyl, alkynyl, acyl, substituted amino, cycloalkyl, carboxy, oxo, hydroxy, alkoxy, aryloxy, halogen, aryl, aralkyl, heteroaryl, heterocyclyl, heteroarylalkyl, heterocyclylalkyl, or —C(═O)QR₇, wherein when Q is O or NH, and R₇ can be alkyl, alkenyl, alkynyl, aryl, aralkyl, cycloalkyl, or heteroarylalkyl; or when Q is NH, R₇ also can be heteroaryl, heterocyclyl or heterocyclylalkyl. In one embodiment, Rₚ and Rⱼ together can join to form a (5-8)-membered cyclic ring, which optionally can be benzofused.

In another embodiment, when R₅ is OR_{z}, and R₄ is OR_{c}, then R_{c} and R_{z} can be joined together to form a six-membered acetal, wherein the carbon joining the oxygen atoms can be substituted with Rₗ and Rₘ, wherein Rₗ and Rₘ can be hydrogen, alkyl, alkenyl, alkynyl, cycloalkyl, aryl or aralkyl; Rₗ and Rₘ together can join to form a cyclic ring or Rₗ and Rₘ together can join to form an oxo,
wherein the ring optionally can contain one or more heteroatoms selected from O, N or S, and the ring optionally can be substituted with one or more of alkyl, alkenyl, alkynyl, acyl, substituted amino, cycloalkyl, carboxy, oxo, hydroxy, alkoxy, aryloxy, halogen, aryl, aralkyl, heteroaryl, heterocyclyl, heteroarylalkyl, heterocyclylalkyl, or —C(═O)QR₇,
   wherein Q can be O or NH, and R₇ can be alkyl, alkenyl, alkynyl, aryl, aralkyl, cycloalkyl, or heteroarylalkyl; or when Q is NH, R₇ also can be heteroaryl, heterocyclyl or heterocyclylalkyl.

The present invention also encompasses illustrative compounds, including:
1-O-decyl-2,3-O-isopropylidene-4-O-{[(phenyl-sulfonyl)-amino]-carbonyl}-6-deoxy-α-L-sorbofuranoside (Compound No. 1);
1-O-Dodecyl-2,3-O-isopropylidene-4-O-{[(4-chloro-phenyl)-sulfonylamino]-carbonyl}-6-deoxy-6-(1-azepanyl)-α-L-sorbofuranoside (Compound No. 2);
1-O-Dodecyl-2,3-O-isopropylidene-4-O-{[phenyl-sulfonylamino]-carbonyl}-6-deoxy-6-(1-azepanyl)-α-L-sorbofuranoside (Compound No. 3);
1-O-Dodecyl-2,3-O-isopropylidene-4-O-{[(4-methyl-phenyl)-sulfonylamino]-carbonyl}-6-deoxy-6-(1-azepanyl)-α-L-sorbofuranoside (Compound No. 4);
1-O-Dodecyl-2,3-O-isopropylidene-4-O-{[(4-methyl-phenyl)-sulfonylamino]-carbonyl}-6-deoxy-6-(4-morpholinyl)-α-L-sorbofuranoside (Compound No. 5);
1-O-Dodecyl-2,3-O-isopropylidene-4-O-{[(4-chloro-phenyl)-sulfonylamino]-carbonyl}-6-deoxy-6-(4-morpholinyl)-α-L-sorbofuranoside (Compound No. 6);
1-O-Dodecyl-2,3-O-isopropylidene-4-O-{[(phenyl-sulfonyl)-amino]-carbonyl}-6-deoxy-6-(4-morpholinyl)-α-L-sorbofuranoside (Compound No. 7);
1-O-Dodecyl-2,3-O-isopropylidene-4-O-{[(phenylsulfonyl)-amino]-carbonyl}-6-deoxy-6-(1-pyrrolidinyl)-α-L-sorbofuranoside (Compound No. 8);
1-O-Dodecyl-2,3-O-isopropylidene-4-O-{[(4-methyl-phenyl)-sulfonylamino]-carbonyl}-6-deoxy-α-L-sorbofuranoside (Compound No. 9);
Hydrochloride salt of 1-O-Dodecyl-2,3-O-isopropylidene-4-O-{[(4-methyl-phenyl)-sulfonylamino]-carbonyl}-6-deoxy-6-(1-pyrrolidinyl)-α-L-sorbofuranoside (Compound No. 10);
Hydrochloride salt of 1-O-Dodecyl-2,3-O-isopropylidene-4-O-{[(4-chloro-phenyl)-sulfonylamino]-carbonyl}-6-deoxy-6-(1-pyrrolidinyl)-α-L-sorbofuranoside (Compound No. 11);
1-O-Heptyl-2,3-O-isopropylidene-4-O-{[4-(2-methoxy-2-oxo-ethyl)-phenyl]-amino}-carbonyl-6-deoxy-6-(1-azepanyl)-α-L-sorbofuranoside (Compound No. 12);
1-O-Heptyl-2,3-O-isopropylidene-4-O-{[4-(2-methoxy-2-oxo-ethyl)-phenyl]-amino}-carbonyl-6-deoxy-6-(4-morpholinyl)-α-L-sorbofuranoside (Compound No. 13);
Hydrochloride salt of 1-O-heptyl-2,3-O-isopropylidene-4-O-{[4-(2-methoxy-2-oxo-ethyl)-phenyl]-amino}-carbonyl-6-deoxy-6-(1-azepanyl)-α-L-sorbofuranoside (Compound No. 14);
1-O-Dodecyl-2,3-O-isopropylidene-4-O-{[4-(2-methoxy-2-oxo-ethyl)-phenyl]-amino}-carbonyl-6-deoxy-6-(1-azepanyl)-α-L-sorbofuranoside (Compound No. 15);
1-O-Dodecyl-2,3-O-isopropylidene-4-O-{[4-(2-methoxy-2-oxo-ethyl)-phenyl]-amino}-carbonyl-6-deoxy-6-(4-morpholinyl)-α-L-sorbofuranoside (Compound No. 16);
1-O-Dodecyl-2,3-O-isopropylidene-4-O-{[4-(2-methoxy-2-oxo-ethyl)-phenyl]-amino}-carbonyl-6-deoxy-6-(1-pyrrolidinyl)-α-L-sorbofuranoside (Compound No. 17);
1-O-Dodecyl-2,3-O-isopropylidene-4-O-{[4-(2-methoxy-2-oxo-ethyl)-phenyl]-amino}-carbonyl-6-deoxy-6-(1-piperidinyl)-α-L-sorbofuranoside (Compound No. 18);
1-O-Heptyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 19);
1-O-Decyl-2,3-O-isopropylidene-4-O-{[(4-[2-hydroxy-2-oxo-ethyl]-phenyl)-amino]-carbonyl}-6-deoxy-6-(1-azepanyl)-α-L-sorbofuranoside (Compound No. 20);
Tris salt of 1-O-Heptyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 21);
Tris salt of-1-O-decyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 22);
1-O-Dodecyl-2,3-O-isopropylidene-4-O-{[4-(2-hydroxy-2-oxo-ethyl)-phenyl]-amino}-carbonyl-6-deoxy-6-(1-piperidinyl)-α-L-sorbofuranoside (Compound No. 23);
1-O-Dodecyl-2,3-O-isopropylidene-4-O-{[4-(2-hydroxy-2-oxo-ethyl)-phenyl]-amino}-carbonyl-6-deoxy-6-(4-morpholinyl)-α-L-sorbofuranoside (Compound No. 24);
1-O-Dodecyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 25);
1-O-Dodecyl-2,3-O-isopropylidene-4-O-{[4-(2-hydroxy-2-oxo-ethyl)-phenyl]-amino}-carbonyl-6-deoxy-6-(1-pyrrolidinyl)-α-L-sorbofuranoside (Compound No. 26);
1-O-Dodecyl-2,3-O-isopropylidene-4-O-[4-(2-hydroxy-2-oxo-phenyl)-amino]-carbonyl-6-deoxy-6-(1-azepanyl)-α-L-sorbofuranoside (Compound No. 27);
Tris salt of 1-O-Decyl-2,3-O-isopropylidene-4-O-[(4-{2-hydroxy-2-oxo-ethyl}-phenyl)-amino]-carbonyl-6-deoxy-6-[2-(1-pyrrolidinyl)-ethyl]-amino-α-L-sorbofuranoside (Compound No. 28);
Tris salt of 1-O-Decyl-2,3-O-isopropylidene-4-O-[{4-(2-hydroxy-2-oxo-ethyl)-phenyl}-amino]-carbonyl-6-deoxy-6-[2-(1-piperidinyl)-ethyl]amino-α-L-sorbofuranoside. (Compound No. 29); Tris salt of 1-O-Decyl-2,3-O-isopropylidene-4-O-{[4-(2-hydroxy-2-oxo-ethyl)-phenyl]-amino}-carbonyl-6-deoxy-6-[2-(4-morpholinyl)-ethyl]-amino-α-L-sorbofuranoside (Compound No. 30);
Tris salt of 1-O-Decyl-2,3-O-isopropylidene-4-O-{[4-(2-hydroxy-2-oxo-ethyl)-phenyl]-amino}-carbonyl-6-deoxy-6-[2-(1-cycloheptyl-amino)-ethyl]-amino-α-L-sorbofuranoside (Compound No. 31);
(4ξ)-1-O-Dodecyl-2,3-O-isopropylidene-4,6-dideoxy-4-[{[(4-fluoro-phenyl)-amino]carbonyl}-amino]-α-L-erythro-hex-2-ulofuranoside (Compound No. 32);
(4ξ)-1-O-Dodecyl-2,3-O-isopropylidene-4,6-dideoxy-4-{[(butyl-amino)-carbonyl]-amino}-α-L-erythro-hex-2-ulofuranoside (Compound No. 33);
(4ξ)-1-O-Dodecyl-2,3-O-isopropylidene-4,6-dideoxy-4-{[(4-fluoro-phenyl)-sulfonyl]-amino}-α-L-erythro-hex-2-ulofuranoside (Compound No. 34);
(4ξ)-1-O-Dodecyl-2,3-O-isopropylidene-4,6-dideoxy-4-{[(4-fluoro-phenyl)-carbonyl]-amino}-α-L-erythro-hex-2-ulofuranoside (Compound No. 35);
(4ξ)-1-O-Dodecyl-2,3-O-isopropylidene-4,6-dideoxy-4-[{[(2-phenylethyl)-amino]-thiocarbonyl}-amino]-α-L-erythro-hex-2-ulofuranoside (Compound No. 36);
(4ξ)-1-O-Dodecyl-2,3-O-isopropylidene-4,6-dideoxy-4-[{[(4-[2-hydroxy-2-oxo-ethyl]-phenyl)-amino]-carbonyl}-amino]-α-L-erythro-hex-2-ulofuranoside (Compound No. 37);
(4ξ)-1-O-Dodecyl-2,3-O-isopropylidene-4,6-dideoxy-4-{[3-(1,3-benzodionol-5-yl)-propanoyl]-amino}-α-L-erythro-hex-2-ulofuranoside (Compound No. 38);
1-O-Dodecyl-2,3-O-isopropylidene-6-deoxy-6-{[(4-fluorophenyl)-amino]-carbonyl}-amino-α-L-sorbofuranoside (Compound No. 39);
1-O-Dodecyl-2,3-O-isopropylidene-6-deoxy-6-[(4-fluorophenyl)-sulfonyl]-amino-α-L-sorbofuranoside (Compound No. 40);
1-O-Dodecyl-2,3-O-isopropylidene-6-deoxy-6-(ethylsulfonyl)-amino-α-L-sorbofuranoside (Compound No. 41);
1-O-Dodecyl-2,3-O-isopropylidene-6-deoxy-6-{(4-fluoro-phenyl)-carbonyl}-amino-α-L-sorbofuranoside (Compound No. 42);
1-O-Dodecyl-2,3-O-isopropylidene-6-deoxy-6-{[3-(1,3-benzodioxol-5-yl)-propanoyl]-amino}-α-L-sorbofuranoside (Compound No. 43);
1-O-Dodecyl-2,3-O-isopropylidene-6-deoxy-6-{[{(4-[2-hydroxy-2-oxo-ethyl]-phenyl)-amino}-carbonyl]-amino}-α-L-sorbofuranoside (Compound No. 44);
1-O-Dodecyl-2,3-O-isopropylidene-6-deoxy-6-{[(butyl-amino)-carbonyl]amino}-α-L-sorbofuranoside (Compound No. 45);
1-O-Dodecyl-2,3-O-isopropylidene-6-deoxy-6-({[(4-fluoro-phenyl)-amino]-thiocarbonyl}-amino)-α-L-sorbofuranoside (Compound No. 46);
Hydrochloride salt of 1-O-Dodecyl-2,3-O-isopropylidene-6-deoxy-6-{1-[4-({4-ethoxy-3-[5-{1-methyl-3-propyl-7-oxo-1,6-dihydro-pyrazolo[4,3-d]-pyrimidinyl}]-phenyl}-sulfonyl)-piperazinyl]}-α-L-sorbofuranoside (Compound No. 47);
1-O-[6-{(4-Nitro-phenyl)-amino-carbonyloxy}-hexyl]-2,3;4,6-di-O-isopropylidene-α-L-sorbofuranoside (Compound No. 48);
1-O-[6-{(4-Chloro-phenyl)-amino-carbonyloxy}-hexyl]-2,3;4,6-di-O-isopropylidene-α-L-sorbofuranoside (Compound No. 49);
1-O-[6-{(4-Methoxy-phenyl-amino-carbonyloxy)-hexyl]-2,3;4,6-di-O-isopropylidene-α-L-sorbofuranoside (Compound No. 50);
1-O-{6-[(4-Methyl-phenyl)-amino-carbonyloxy}-hexyl]-2,3;4,6-di-O-isopropylidene-α-L-sorbofuranoside (Compound No. 51);
(4ξ)-1-O-Dodecyl-2,3-O-isopropylidene-4-C-methyl-6-deoxy-α-L-erythro-hex-2-ulofuranoside (Compound No. 52);

(4ξ)-1-O-Dodecyl-2,3-O-isopropylidene-4-C-methyl-4-O-{[(phenylsulfonyl)-amino]-carbonyl}-6-deoxy-α-L-erythro-hex-2-ulofuranoside (Compound No. 53);
(4ξ)-1-O-Dodecyl-2,3-O-isopropylidene-4-C-methyl-4-O-[{[(4-methyl-phenyl)-sulfonyl]-amino}-carbonyl]-6-deoxy-α-L-erythro-hex-2-ulofuranoside (Compound No. 54);
(4ξ)-1-O-Dodecyl-2,3-O-isopropylidene-4-C-methyl-4-O-[{[(4-chloro-phenyl)-sulfonyl]-amino}-carbonyl]-6-deoxy-α-L-erythro-hex-2-ulofuranoside (Compound No. 55);
(4ξ)-1-O-Dodecyl-2,3-O-isopropylidene-4-C-methyl-4-O-{[{2,5-dichloro-phenyl)-sulfonyl}-amino]-carbonyl}-6-deoxy-α-L-erythro-hex-2-ulofuranoside (Compound No. 56);
(4ξ)-1-O-Dodecyl-2,3-O-isopropylidene-4-C-methyl-4-O-[{[(2-methyl-phenyl)-sulfonyl]-amino}-carbonyl]-6-deoxy-α-L-erythro-hex-2-ulofuranoside (Compound No. 57);
(4ξ)-1-O-Dodecyl-2,3-O-isopropylidene-4-C-heptyl-4-O-[2-(1-piperidinyl)-ethyl]-6-deoxy-α-L-erythro-hex-2-ulofuranoside (Compound No. 58);
(4ξ)-1-O-Dodecyl-2,3-O-isopropylidene-4-C-heptyl-4-O-[2-(1-azepanyl)-ethyl]-6-deoxy-α-L-erythro-hex-2-ulofuranoside (Compound No. 59);
(4ξ)-1-O-Dodecyl-2,3-O-isopropylidene-4-C-heptyl-4-O-[2-(1-morpholinyl)-ethyl]-6-deoxy-α-L-erythro-hex-2-ulofuranoside (Compound No. 60);
(4ξ)-1-O-Dodecyl-2,3-O-isopropylidene-4-C-heptyl-4-O-[2-(1-pyrrolidinyl)-ethyl]-6-deoxy-α-L-erythro-hex-2-ulofuranoside (Compound No. 61);
(4ξ)-1-O-Dodecyl-2,3-O-isopropylidene-4-C-heptyl-4-O-heptyl-6-deoxy-α-L-erythro-hex-2-ulofuranoside (Compound No. 62);
(4ξ)-1-O-Dodecyl-2,3-O-isopropylidene-4-C-heptyl-4-O-[2-(1-dimethylamino)-propyl]-6-deoxy-α-L-erythro-hex-2-ulofuranoside (Compound No. 63);
(4ξ)-1-O-Dodecyl-2,3-O-isopropylidene-4-C-methyl-4-O-[{[(2-azepanyl)-sulfonyl]-amino}-carbonyl]-6-deoxy-α-L-erythro-hex-2-ulofuranoside (Compound No. 64);
1-O-Heptyl-2,3-O-isopropylidene-6-O-{[(4-methyl-phenyl)-amino]-carbonyl}-α-L-sorbofuranoside (Compound No. 65);
1-O-Dodecyl-2,3-O-isopropylidene-6-deoxy-6-[({[2-(carboxymethyl)phenyl]amino}-carbonyl)amino]-α-L-sorbofuranoside (Compound No. 66);
1-O-Dodecyl-2,3-O-isopropylidene-6-deoxy-6-[({[3-(carboxymethyl)phenyl]amino}-carbonyl)amino]-α-L-sorbofuranoside (Compound No. 67);
1-O-Dodecyl-2,3-O-isopropylidene-6-deoxy-6-{[(4-trifluoromethyl)benzoyl]amino}-α-L-sorbofuranoside (Compound No. 68);
1-O-Dodecyl-2,3-O-isopropylidene-6-deoxy-6-{[(4-fluorophenyl)acetyl}amino]-α-L-sorbofuranoside (Compound No. 69); 1-O-Dodecyl-2,3-O-isopropylidene-6-deoxy-6-{(3-fluorobenzoyl)amino}-α-L-sorbofuranoside (Compound No. 70);
1-O-Dodecyl-2,3-O-isopropylidene-6-deoxy-6-{(quinolin-2-ylcarbonyl)amino}-α-L-sorbofuranoside (Compound No. 71);
1-O-Dodecyl-2,3-O-isopropylidene-6-deoxy-6-{(2-thienylacetyl)amino}-α-L-sorbofuranoside (Compound No. 72);
1-O-Dodecyl-2,3-O-isopropylidene-6-deoxy-6-{[(4-methoxyphenyl)acetyl]amino}-α-L-sorbofuranoside (Compound No. 73);
1-O-Dodecyl-2,3-O-isopropylidene-6-deoxy-6-{[(3-fluorophenyl)acetyl]amino}-α-L-sorbofuranoside (Compound No. 74);
1-O-Dodecyl-2,3-O-isopropylidene-6-deoxy-6-{(3,4-dimethoxybenzoyl)amino}-α-L-sorbofuranoside (Compound No. 75);
1-O-Dodecyl-2,3-O-isopropylidene-6-deoxy-6-{(isoquinolin-1-ylcarbonyl)amino}-α-L-sorbofuranoside (Compound No. 76);
1-O-Dodecyl-2,3-O-isopropylidene-6-deoxy-6-{[4-(acetylamino)benzoyl]amino}-α-L-sorbofuranoside (Compound No. 77);
1-O-Dodecyl-2,3-O-isopropylidene-6-deoxy-6-{[(pyridin-4-yl)-carbonyl]-amino}-α-L-sorbofuranoside (Compound No. 78);
1-O-Dodecyl-2,3-O-isopropylidene-6-deoxy-6-{[(2,6-dichloropyridin-4-yl)-carbonyl]-amino}-α-L-sorbofuranoside (Compound No. 79);
1-O-Dodecyl-2,3-O-isopropylidene-6-deoxy-6-{[(quinolin-3-yl)-carbonyl]-amino}-α-L-sorbofuranoside (Compound No. 80);
1-O-Dodecyl-2,3-O-isopropylidene-6-deoxy-6-{[(5-methyl-3-phenylisoxazol-4-yl)-carbonyl]-amino}-α-L-sorbofuranoside (Compound No. 81);
1-O-Dodecyl-2,3-O-isopropylidene-6-deoxy-6-{(phenyl)acetyl}-amino-α-L-sorbofuranoside (Compound No. 82);
1-O-Dodecyl-2,3-O-isopropylidene-6-deoxy-6-{[(4-chlorophenyl)acetyl]amino-α-L-sorbofuranoside (Compound No. 83);
1-O-Dodecyl-2,3-O-isopropylidene-6-deoxy-6-{[(3,5-difluorophenyl)acetyl]amino}-α-L-sorbofuranoside (Compound No. 84);
1-O-Dodecyl-2,3-O-isopropylidene-6-deoxy-6-{[(3-methoxyphenyl)acetyl]amino}-α-L-sorbofuranoside (Compound No. 85);
1-O-Dodecyl-2,3-O-isopropylidene-6-deoxy-6-{[(3-chlorophenyl)acetyl]amino}-α-L-sorbofuranoside (Compound No. 86);
1-O-Dodecyl-2,3-O-isopropylidene-6-deoxy-6-{[(2-methoxyphenyl)acetyl]amino}-α-L-sorbofuranoside (Compound No. 87);
1-O-Dodecyl-2,3-O-isopropylidene-6-deoxy-6-{[(2,4-difluorophenyl)acetyl]amino}-α-L-sorbofuranoside (Compound No. 88);
1-O-Dodecyl-2,3-O-isopropylidene-6-deoxy-6-{[(2,6-chlorophenyl)acetyl]amino}-α-L-sorbofuranoside (Compound No. 89);
1-O-Dodecyl-2,3-O-isopropylidene-6-deoxy-6-{[(4-methylphenyl)acetyl]amino}-α-L-sorbofuranoside (Compound No. 90);
1-O-Dodecyl-2,3-O-isopropylidene-6-deoxy-6-{[(3,4-difluorophenyl)acetyl]amino}-α-L-sorbofuranoside (Compound No. 91);
1-O-Dodecyl-2,3-O-isopropylidene-6-deoxy-6-{[(2,5-difluorophenyl)acetyl]amino}-α-L-sorbofuranoside (Compound No. 92);
1-O-Dodecyl-2,3-O-isopropylidene-6-deoxy-6-{[(2,4,5-trifluorophenyl)acetyl]amino}-α-L-sorbofuranoside (Compound No. 93);
1-O-Dodecyl-2,3-O-isopropylidene-6-deoxy-6-{[(3,4-dichlorophenyl)acetyl]amino}-α-L-sorbofuranoside (Compound No. 94);
1-O-Dodecyl-2,3-O-isopropylidene-6-deoxy-6-{[(4-hydroxyphenyl)acetyl]amino}-α-L-sorbofuranoside (Compound No. 95);
1-O-Dodecyl-2,3-O-isopropylidene-6-deoxy-6-{[(2-methylphenyl)acetyl]amino}-α-L-sorbofuranoside (Compound No. 96);
1-O-Dodecyl-2,3-O-isopropylidene-6-deoxy-6-{[(2-chlorophenyl)acetyl]amino}-α-L-sorbofuranoside (Compound No. 97);
1-O-Dodecyl-2,3-O-isopropylidene-6-deoxy-6-[(1,3-benzodioxol-5-ylacetyl)amino]-L-sorbofuranoside (Compound No. 98);
1-O-Dodecyl-2,3-O-isopropylidene-6-deoxy-6-{[(3-hydroxyphenyl)acetyl]amino}-α-L-sorbofuranoside (Compound No. 99);
1-O-Dodecyl-2,3-O-isopropylidene-6-deoxy-6-{[(4-hydroxy-3-fluorophenyl)acetyl]amino}-α-L-sorbofuranoside (Compound No. 100);
1-O-Dodecyl-2,3-O-isopropylidene-6-deoxy-6-{[(4-isopropylphenyl)acetyl]amino}-α-L-sorbofuranoside (Compound No. 101);
1-O-Dodecyl-2,3-O-isopropylidene-6-deoxy-6-{[biphenyl-4-ylacetyl]amino}-α-L-sorbofuranoside (Compound No. 102);
1-O-Dodecyl-2,3-O-isopropylidene-6-deoxy-6-{[(3-methylphenyl)acetyl]amino}-α-L-sorbofuranoside (Compound No. 103);
1-O-Dodecyl-2,3-O-isopropylidene-6-deoxy-6-{[(2-fluoro-6-chlorophenyl)acetyl]amino}-α-L-sorbofuranoside (Compound No. 104);
1-O-Dodecyl-2,3-O-isopropylidene-6-deoxy-6-{[(2-chloro-4-fluorophenyl)acetyl]amino}-α-L-sorbofuranoside (Compound No. 105);
1-O-Dodecyl-2,3-O-isopropylidene-6-deoxy-6-{[(4-trifluoromethoxyphenyl)acetyl]amino}-α-L-sorbofuranoside (Compound No. 106);
1-O-Dodecyl-2,3-O-isopropylidene-6-deoxy-6-{[(2-trifluoromethoxyphenyl)acetyl]amino}-α-L-sorbofuranoside (Compound No. 107);
1-O-Dodecyl-2,3-O-isopropylidene-4-O-methyl-6-deoxy-6-{[(4-fluorophenyl)acetyl]amino}-α-L-sorbofuranoside (Compound No. 108);
1-O-(2-butoxyethyl)-2,3-O-isopropylidene-6-deoxy-6-{[(3,4-difluorophenyl) acetyl]amino}-α-L-sorbofuranose (Compound No. 109);
1-O-(2-butoxyethyl)-2,3-O-isopropylidene-6-deoxy-6-{[(3,4 dichlorophenyl)acetyl]amino}-α-L-sorbofuranose (Compound No. 110);
1-O-(2-butoxyethyl)-2,3-O-isopropylidene-6-deoxy-6-{[(4-methoxyphenyl)acetyl]amino}-α-L-sorbofuranose (Compound No. 111);
1-O-(2-butoxyethyl)-2,3-O-isopropylidene-6-deoxy-6-{[(3-methoxyphenyl)acetyl]amino}-α-L-sorbofuranose (Compound No. 112);
1-O-(2-butoxyethyl)-2,3-O-isopropylidene-6-deoxy-6-{[(4-fluorophenyl)acetyl]amino}-α-L-sorbofuranoside (Compound No. 113);
(4ξ)-1-O-Heptyl-2,3-O-isopropylidene-4-C-methyl-4-O-[{[(4-methylphenyl)sulphonyl]amino}-carbonyl]-6-deoxy-α-L-erythro-hex-2-ulofuranoside (Compound No. 114);
(4ξ)-1-O-Heptyl-2,3-O-isopropylidene-4-C-methyl-4-O-[{[(2-methylphenyl)sulphonyl]amino}-carbonyl]-6-deoxy-α-L-erythro-hex-2-ulofuranoside (Compound No. 115);
(4ξ)-1-O-Heptyl-2,3-O-isopropylidene-4-C-methyl-4-O-[{[(4-chlorophenyl)sulphonyl]amino}-carbonyl]-6-deoxy-α-L-erythro-hex-2-ulofuranoside (Compound No. 116);
(4ξ)-1-O-Heptyl-2,3-O-isopropylidene-4-C-methyl-4-O-[{[phenylsulphonyl]amino}carbonyl]-6-deoxy-α-L-erythro-hex-2-ulofuranoside (Compound No. 117); or
(4ξ)-1-O-Heptyl-2,3-O-isopropylidene-4-C-methyl-4-O-[{[(4-fluorophenyl)sulphonyl]amino}-carbonyl]-6-deoxy-α-L-erythro-hex-2-ulofuranoside (Compound No. 118).

The present invention also encompasses processes for preparing compounds of Formula IV comprising

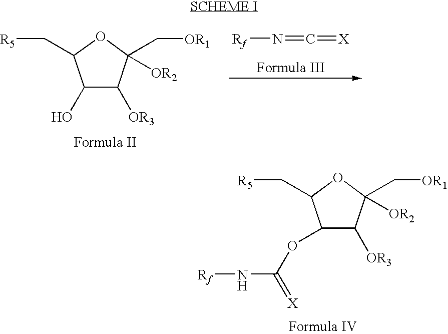

reacting a compound of Formula II with a compound of Formula III to form a compound of Formula IV,
wherein R₁ can be alkyl, alkenyl, alkynyl, cycloalkyl, aryl, heterocyclyl, heteroaryl, heterocyclylalkyl, heteroarylalkyl, aralkyl or —(CH₂)ₙO(C═O)NHRₓ,
   wherein n can be an integer 2-10, and
   Rₓ can be alkyl, alkenyl, alkynyl, cycloalkyl, aryl, aralkyl, heteroaryl, heterocyclyl, heterocyclylalkyl or heteroarylalkyl;
R₂ and R₃ together can form a five-membered acetal, wherein the carbon joining the two oxygen atoms can be substituted with Rₗ and Rₘ,
   wherein Rₗ and Rₘ can be hydrogen, alkyl, alkenyl, alkynyl, cycloalkyl, aryl or aralkyl; Rₗ and Rₘ together can join to form a cyclic ring; or Rₗ and Rₘ together can join to form an oxo,
      wherein the ring optionally can contain one or more heteroatoms selected from O, N or S, and the ring optionally can be substituted with one or more of alkyl, alkenyl, alkynyl, acyl, substituted amino, cycloalkyl, carboxy, oxo, hydroxy, alkoxy, aryloxy, halogen, aryl, aralkyl, heteroaryl, heterocyclyl, heteroarylalkyl, heterocyclylalkyl, or —C(═O)QR₇,
         wherein Q can be O or NH, and R₇ can be alkyl, alkenyl, alkyl, aryl, aralkyl, cycloalkyl, or heteroarylalkyl; or when Q is NH, R₇ also can be heteroaryl, heterocyclyl or heterocyclylalkyl; or
R₂ and R₃, instead of forming an acetal, optionally and independently can be lower (C₁-C₄)-alkyl, (CH₂)ₖ-aryl, —C(═R_{y})NHRₓ or acyl,
   wherein k can be an integer from 1-4,
   R_{y} can be O or S, and
   Rₓ can be alkyl, alkenyl, alkynyl, cycloalkyl, aryl, aralkyl, heteroaryl, heterocyclyl, heterocyclylalkyl or heteroarylalkyl; and
R₅ can be hydrogen, alkyl, cycloalkyl, heteroaryl, heterocyclyl, —NRₚRⱼ, OR_{z}, —NHC(═O)ORₛ, —NHYR_{d}, —NHC(=T)NRₜRₓ or —(CH₂)_{w}(C═O)NRₐR_{b}, wherein
   Rₚ and Rⱼ independently can be hydrogen, alkyl, cycloalkyl, aryl, heteroaryl, heterocyclyl, heteroarylalkyl, heterocyclylalkyl, or aralkyl, or Rₚ and Rⱼ together can join to form a cyclic ring, which optionally can be benzofused, containing 0-4 heteroatom selected from 0-4 heteroatoms selected from O, S or N wherein the ring can be substituted with one or more of alkyl, alkenyl, alkynyl, amino, substituted amino, cycloalkyl, carboxy, oxo, hydroxy, alkoxy, aryloxy, halogen, aryl, aralkyl, heteroaryl, heterocyclyl, heteroarylalkyl or heterocyclylalkyl;
   R_{z} can be alkyl, alkenyl, alkynyl, cycloalkyl, aryl, aralkyl, heteroaryl, heterocyclyl, heteroarylalkyl, heterocyclylalkyl, acyl or —C(═O)NR_{f}R_{q},
      wherein R_{f} and R_{q} each independently can be hydrogen, allyl, alkenyl, alkynyl, cycloalkyl, aryl, aralkyl, heterocyclyl, heteroaryl, heteroarylalkyl, heterocyclylalkyl or S(O)₂R₆; or R_{f} and R_{q} together can form a ring,
         wherein R₆ can be alkyl, alkenyl, alkynyl, cycloalkyl, aryl, aralkyl, heteroaryl, heterocyclyl, heterocyclylalkyl, heteroaryl-alkyl or substitute amino;
   Rₛ can be alkyl, alkenyl, alkynyl, cycloalkyl, aryl, aralkyl, heterocyclylalkyl or heteroarylalkyl,
   Y can be —C(═O), —C(═S) or SO₂),
   R_{d} can be alkyl, alkenyl, alkynyl, cycloalkyl, aryl, aralkyl, heteroaryl, heterocyclyl, heteroarylalkyl or heterocyclylalkyl,
   T can be O, S, —N(CN), —N(NO₂), or —CH(NO₂),
   Rₜ can be H, OH or Rₓ,
   Rₓ can be alkyl, alkenyl, alkynyl, cycloalkyl, aryl, aralkyl, heteroaryl, heterocyclyl, heterocyclylalkyl or heteroarylalkyl,
   w can be 1-4, and
   Rₐ and R_{b} each independently can be hydrogen or R_{d}, or Rₐ and R_{b}, together with the nitrogen atom carrying them, can be the N-terminus of an amino acid or di-tetrapeptide,
      wherein R_{f} and R_{q} each independently can be hydrogen, alkyl, alkenyl, alkynyl, cycloalkyl, aryl, aralkyl, heterocyclyl, heteroaryl, heteroarylalkyl, heterocyclylalkyl or S(O)₂R₆; or R_{f} and R_{q} together can form a ring,
         wherein R₆ can be alkyl, alkenyl, alkynyl, cycloalkyl, aryl, aralkyl, heteroaryl, heterocyclyl, heterocyclylalkyl, heteroarylalkyl or substituted amino.

The present invention also encompasses processes for preparing compounds of Formula X or compounds of Formula XII comprising the steps of:

a. oxidizing a compound of Formula V

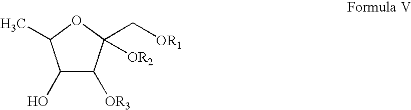

to form a compound of Formula VI;

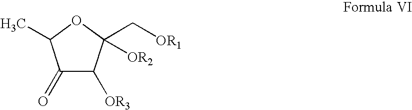

b. reacting the compound of Formula VI with hydroxylamine hydrochloride to form a compound of Formula VII;

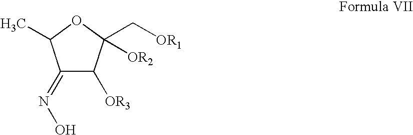

c. reducing the compound of Formula VII to form a compound of Formula VIII;

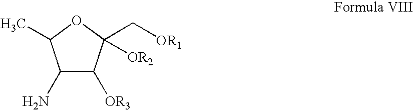

and

d. reacting the compound of Formula VIII with a compound of Formula IX

L-Y—R_{d} Formula IX

to form a compound of Formula X

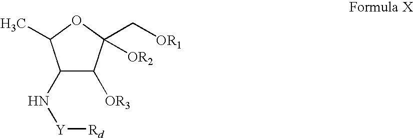

or

reacting the compound of Formula VIII with a compound of Formula III

X═C═NR_{f} Formula III

or a compound of Formula XI

ArOC(═X)NHR_{f} Formula XI

to form a compound of Formula XII

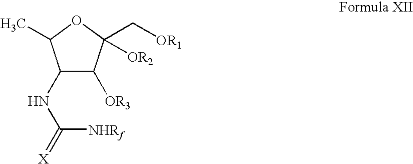

wherein
R₁ can be alkyl, alkenyl, alkynyl, cycloalkyl, aryl, heterocyclyl, heteroaryl, heterocyclylalkyl, heteroarylalkyl, aralkyl or —(CH₂)ₙO(C═O)NHRₓ,
   wherein n can be an integer 2-10, and
   Rₓ can be alkyl, alkenyl, alkynyl, cycloalkyl, aryl, aralkyl, heteroaryl, heterocyclyl, heterocyclylalkyl or heteroarylalkyl;
R₂ and R₃ together can form a five-membered acetal, wherein the carbon joining the two oxygen atoms can be substituted with Rₗ and Rₘ,
   wherein Rₗ and Rₘ can be hydrogen, alkyl, alkenyl, alkynyl, cycloalkyl, aryl or aralkyl; Rₗ and Rₘ together can join to form a cyclic ring; or Rₗ and Rₘ together can join to form an oxo,
      wherein the ring optionally can contain one or more heteroatoms selected from O, N or S, and the ring optionally can be substituted with one or more of alkyl, alkenyl, alkynyl, acyl, substituted amino, cycloalkyl, carboxy, oxo, hydroxy, alkoxy, aryloxy, halogen, aryl, aralkyl, heteroaryl, heterocyclyl, heteroarylalkyl, heterocyclylalkyl, or —C(═O)QR₇,
         wherein Q can be O or NH, and R₇ can be alkyl, alkenyl, alkynyl, aryl, aralkyl, cycloalkyl, or heteroarylalkyl; or when Q is NH, R₇ also can be heteroaryl, heterocyclyl or heterocyclylalkyl; or
R₂ and R₃, instead of forming an acetal, optionally and independently can be lower (C₁-C₄)-alkyl, (CH₂)ₖ-aryl, —C(═R_{y})NHRₓ or acyl,
   wherein k can be an integer from 1-4,
   R_{y} can be O or S, and
   Rₓ can be alkyl, alkenyl, alkynyl, cycloalkyl, aryl, aralkyl, heteroaryl, heterocyclyl, heterocyclylalkyl or heteroarylalkyl;
Y can be —C(═O), —C(═S) or SO₂);
R_{d} can be alkyl, alkenyl, alkynyl, cycloalkyl, aryl, aralkyl, heteroaryl, heterocyclyl, heteroarylalkyl or heterocyclylalkyl;
L can be a leaving group;
R_{f} can be hydrogen, alkyl, alkenyl, alkynyl, cycloalkyl, aryl, aralkyl, heterocyclyl, heteroaryl, heteroarylalkyl, heterocyclylalkyl or S(O)₂R₆, wherein R₆ can be alkyl, alkenyl, alkynyl, cycloalkyl, aryl, aralkyl, heteroaryl, heterocyclyl, heterocyclylalkyl, heteroarylalkyl or substituted amino; and
X can be O or S.

In one embodiment, the oxidation of the compound of Formula V to form a compound of Formula VI can be carried out in the presence of at least one oxidation agent, for example, pyridinium dichromate; pyridinium chlorochromate; dimethylsulfoxide in combination with acetic anhydride, oxalyl chloride, or trifluoroacetic anhydride; periodinane; or a mixture thereof. In another embodiment, the reaction of the compound of Formula VI with hydroxylamine hydrochloride can be carried out in the presence of at least one base, for example, pyridine, diisopropylethylamine, triethylamine, or a mixture thereof.

In another embodiment, the reduction of the compound of Formula VII can be carried out the presence of at least one reducing agent, for example, lithium aluminum hydride, sodium borohydride, or a mixture thereof. In yet another embodiment, the reaction of the compound of Formula VIII with a compound of Formula IX can be carried out in the presence of at least one base.

In one embodiment, wherein Y is C═O and L is OH, the reaction of the compound of Formula VIII with a compound of Formula IX can proceed via the formation of an activated derivative of a carboxylic acid as intermediate. In another embodiment, the reaction of the compound of Formula VIII with a compound of Formula IX can be carried out in the presence of at least one condensing agent, for example, 1-(3-dimethylaminopropyl)-3-ethyl carbodiimide hydrochloride, dicyclohexylcarbodiimide, or a mixture thereof. In yet another embodiment, the reaction of the compound of Formula VIII with a compound of Formula IX proceeds in the presence of at least one base, for example, N-methylmorpholine, diisopropylamine, triethylamine or a mixture thereof.

In another embodiment, wherein Y is C═O and L is OH, the reaction of the compound of Formula VIII with a compound of Formula IX can proceed via utilizing a mixed anhydride, which comprises reacting the compound of Formula IX with a chloroformate, for example, ethyl chloroformate or isobutylchloroformate.

In one embodiment, wherein Y is C═O or SO₂ and L is Cl, the reaction of the compound of Formula VIII with a compound of Formula IX can proceed in the presence of a base, for example, pyridine, triethylamine, diisopropylethylamine or a mixture thereof.

The present invention also encompasses processes for preparing compounds of Formula XVII or a compound of Formula XVIII comprising the steps of:

a. reacting a compound of Formula XIII

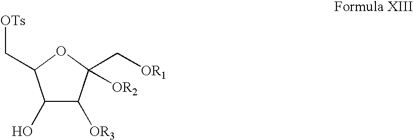

with a compound of Formula XIV

H₂N—P Formula XIV

to form a compound of Formula XV;

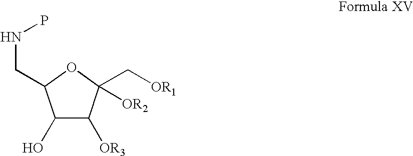

b. deprotecting the compound of Formula XV to form a compound of Formula XVI;

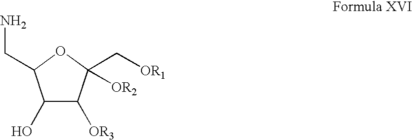

or

a. reacting a compound of Formula XIII with sodium azide to form a compound of formula XIIIa

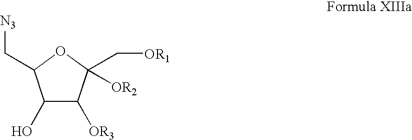

b. reacting the compound of Formula XIIIa with a compound of Formula XIIIb

R₈I

to form a compound of Formula XIIIc

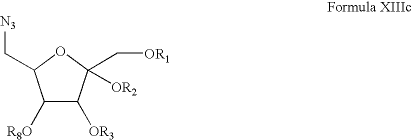

c. reducing the compound of Formula XIIIc to form a compound of Formula XVI and

d. reacting the compound of Formula XVI with a compound of Formula IX

L-Y—R_{d} Formula IX

to form a compound of Formula XVII

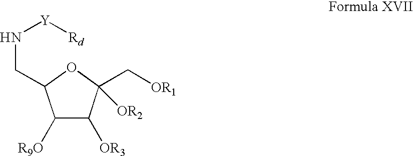

or

reacting the compound of Formula XVI with a compound of Formula III or a compound of Formula XI

X═C═N—R_{f} Formula III

ArO(═X)NHR_{f} Formula XI

to form a compound of Formula XVIII

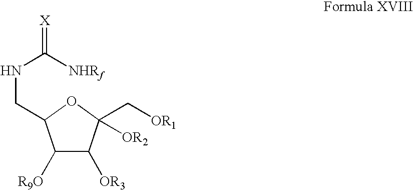

wherein
R₁ can be alkyl, alkenyl, alkynyl, cycloalkyl, aryl, heterocyclyl, heteroaryl, heterocyclylalkyl, heteroarylalkyl, aralkyl or —(CH₂)ₙO(C═O)NHRₓ,
   wherein n can be an integer 2-10, and
   Rₓ can be alkyl, alkenyl, alkynyl, cycloalkyl, aryl, aralkyl, heteroaryl, heterocyclyl, heterocyclylalkyl or heteroarylalkyl;
R₂ and R₃ together can form a five-membered acetal, wherein the carbon joining the two oxygen atoms can be substituted with Rₗ and Rₘ,
   wherein Rₗ and Rₘ can be hydrogen, alkyl, alkenyl, alkynyl, cycloalkyl, aryl or aralkyl; Rₗ and Rₘ together can join to form a cyclic ring; or Rₗ and Rₘ together can join to form an oxo,
      wherein the ring optionally can contain one or more heteroatoms selected from O, N or S, and the ring optionally can be substituted with one or more of alkyl, alkenyl, alkynyl, acyl, substituted amino, cycloalkyl, carboxy, oxo, hydroxy, alkoxy, aryloxy, halogen, aryl, aralkyl, heteroaryl, heterocyclyl, heteroarylalkyl, heterocyclylalkyl, or —C(═O)QR₇,
         wherein Q can be O or NH, and R₇ can be alkyl, alkenyl, alkynyl, aryl, aralkyl, cycloalkyl, or heteroarylalkyl; or when Q is NH, R₇ also can be heteroaryl, heterocyclyl or heterocyclylalkyl; or
R₂ and R₃, instead of forming an acetal, optionally and independently can be lower (C₁-C₄)-alkyl, (CH₂)ₖ-aryl, —C(═R_{y})NHRₓ or acyl,
   wherein k can be an integer from 1-4,
   R_{y} can be O or S, and
   Rₓ can be alkyl, alkenyl, alkynyl, cycloalkyl, aryl, aralkyl, heteroaryl, heterocyclyl, heterocyclylalkyl or heteroarylalkyl;
P can be a protecting group, for example aralkyl or acyl;
Y can be —C(═O), —C(═S) or SO₂);
R_{d} can be alkyl, alkenyl, alkynyl, cycloalkyl, aryl, aralkyl, heteroaryl, heterocyclyl, heteroarylalkyl or heterocyclylalkyl;
L can be a leaving group;
R₉ is hydrogen or alkyl;
R_{f} can be hydrogen, alkyl, alkenyl, alkynyl, cycloalkyl, aryl, aralkyl, heterocyclyl, heteroaryl, heteroarylalkyl, heterocyclylalkyl or S(O)₂R₆, wherein R₆ can be alkyl, alkenyl, alkynyl, cycloalkyl, aryl, aralkyl, heteroaryl, heterocyclyl, heterocyclylalkyl, heteroarylalkyl or substituted amino; and
X can be O or S.

In one embodiment, the deprotection of the compound of Formula XIV can be carried out under deprotection conditions selected from hydrogenation in the presence of palladium on carbon, or catalytic transfer hydrogenation in the presence of ammonium formate and palladium on carbon. This reaction can be carried out in the presence of at least one base.

In another embodiment, wherein Y is C═O and L is OH, the reaction of the compound of Formula XVI with a compound of Formula IX can proceed via the formation of an activated derivative of a carboxylic acid as intermediate. This reaction can be carried out in the presence of at least one condensing agent, for example, 1-(3-dimethylaminopropyl)-3-ethyl carbodiimide hydrochloride, dicyclohexylcarbodiimide, or a mixture thereof. This reaction also can proceed in the presence of a base, for example, N-methylmorpholine, diisopropylamine, triethylamine or a mixture thereof. Further, this reaction can proceed via utilizing a mixed anhydride, which comprises utilizing a mixed anhydride by reacting the compound of Formula IX with a chloroformate, for example, ethyl chloroformate or isobutylchloroformate.

In one embodiment, wherein Y is C═O or SO₂ and L is Cl, the reaction of the compound of Formula XVI with a compound of Formula IX can proceed in the presence of a base, for example, pyridine, triethylamine, diisopropylethylamine or a mixture thereof.

The present invention further encompasses processes for preparing compounds of Formula XXIII comprising the steps of:

a. reacting a compound of Formula XIII

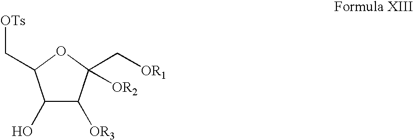

with a compound of Formula XIX

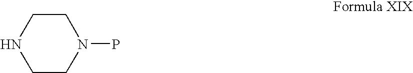

to form a compound of Formula XX;

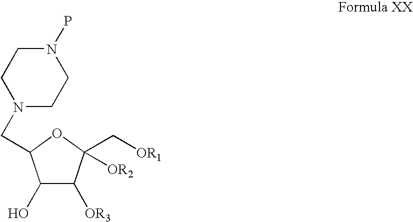

b. deprotecting the compound of Formula XX to form a compound of Formula XXI;

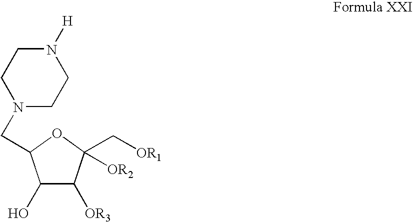

c. reacting the compound of Formula XXI with a compound of Formula XXII

hal-SO₂Rₓ Formula XXII

to form a compound of Formula XXIII

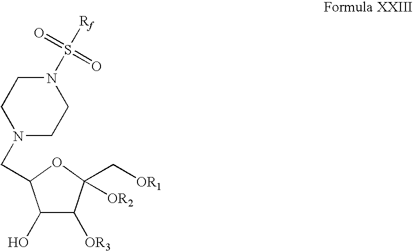

wherein
R₁ can be alkyl, alkenyl, alkynyl, cycloalkyl, aryl, heterocyclyl, heteroaryl, heterocyclylalkyl, heteroarylalkyl, aralkyl or —(CH₂)ₙO(C═O)NHRₓ,
   wherein n can be an integer 2-10, and
   Rₓ can be alkyl, alkenyl, alkynyl, cycloalkyl, aryl, aralkyl, heteroaryl, heterocyclyl, heterocyclylalkyl or heteroarylalkyl;
R₂ and R₃ together can form a five-membered acetal, wherein the carbon joining the two oxygen atoms can be substituted with Rₗ and Rₘ,
   wherein Rₗ and Rₘ can be hydrogen, alkyl, alkenyl, alkynyl, cycloalkyl, aryl or aralkyl; Rₗ and Rₘ together can join to form a cyclic ring; or Rₗ and Rₘ together can join to form an oxo,
      wherein the ring optionally can contain one or more heteroatoms selected from O, N or S, and the ring optionally can be substituted with one or more of alkyl, alkenyl, alkynyl, acyl, substituted amino, cycloalkyl, carboxy, oxo, hydroxy, alkoxy, aryloxy, halogen, aryl, aralkyl, heteroaryl, heterocyclyl, heteroarylalkyl, heterocyclylalkyl, or —C(═O)QR₇,
         wherein Q can be O or NH, and R₇ can be alkyl, alkenyl, alkynyl, aryl, aralkyl, cycloalkyl, or heteroarylalkyl; or when Q is NH, R₇ also can be heteroaryl, heterocyclyl or heterocyclylalkyl; or
R₂ and R₃, instead of forming an acetal, optionally and independently can be lower (C₁-C₄)-alkyl, (CH₂)ₖ-aryl, —C(═R_{y})NHRₓ or acyl,
   wherein k can be an integer from 1-4,
   R_{y} can be O or S, and
   Rₓ can be alkyl, alkenyl, alkynyl, cycloalkyl, aryl, aralkyl, heteroaryl, heterocyclyl, heterocyclylalkyl or heteroarylalkyl;
Hal can be halogen; and
P can be a protecting group, for example, aralkyl or acyl.
In one embodiment, the deprotection of the compound of Formula XX can be carried out under deprotection conditions selected from hydrogenation in the presence of palladium on carbon, or catalytic transfer hydrogenation in the presence of ammonium formate and palladium on carbon.

In another embodiment, the reaction of the compound of Formula XXI with a compound of Formula XXII can be carried out in the presence of a base, for example, pyridine, triethylamine, diisopropylethylamine, or a mixture thereof.

The present invention also encompasses processes for preparing compounds of Formula XXIX comprising the steps of:

a. reacting a compound of Formula XXV

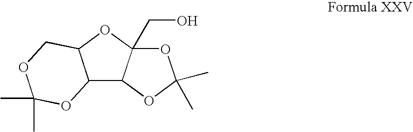

with a compound of Formula XXVI

hal-(CH₂)ₘOH Formula XXVI

to form a compound of Formula XXVII;

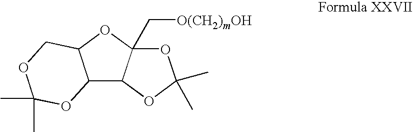

and

b. reacting the compound of Formula XXVII with a compound of Formula XXVIII

R_{f}—N═C═O Formula XXVIII

to form a compound of Formula XXIX

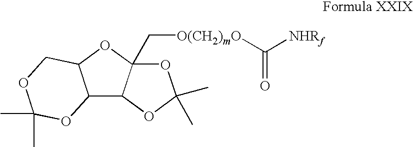

wherein
hal can be halogen;
m can be an integer from 0 to 2; and
R_{f} can be hydrogen, alkyl, alkenyl, alkynyl, cycloalkyl, aryl, aralkyl, heterocyclyl, heteroaryl, heteroarylalkyl, heterocyclylalkyl or S(O)₂R₆, wherein R₆ can be alkyl, alkenyl, alkynyl, cycloalkyl, aryl, aralkyl, heteroaryl, heterocyclyl, heterocyclylalkyl, heteroarylalkyl or substituted amino.

In one embodiment, the reaction of the compound of Formula XXV with a compound of Formula XXVII can be carried out in the presence of a base, for example, potassium hydroxide, sodium hydroxide, or a mixture thereof. In another embodiment, the reaction of the compound of Formula XXV with a compound of Formula XXVII can be carried out in the presence of a phase transfer catalyst, for example, tetrabutylammonium iodide, tetrabutylammonium bromide, or a mixture thereof.

The present invention further encompasses processes for preparing compounds of Formula XXXI comprising the steps of:

a. oxidizing the compound of Formula V

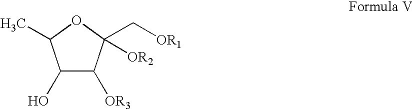

to form a compound of Formula VI;

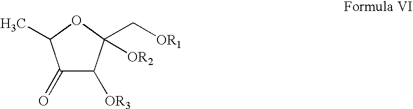

b. reacting the compound of Formula VI with a Grignard reagent to form a compound Formula XXX;

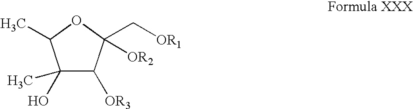

and

c. reacting the compound of Formula XXX with a compound of Formula XXVIII

O═C═N—R_{f} Formula XXVIII

to form a compound of Formula XXXI

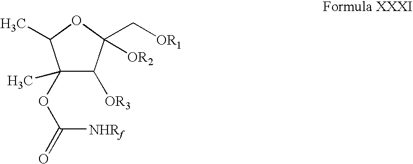

wherein
R_{f} can be hydrogen, alkyl, alkenyl, alkynyl, cycloalkyl, aryl, aralkyl, heterocyclyl, heteroaryl, heteroarylalkyl, heterocyclylalkyl or S(O)₂R₆, wherein R₆ can be alkyl, alkenyl, alkynyl, cycloalkyl, aryl, aralkyl, heteroaryl, heterocyclyl, heterocyclylalkyl, heteroarylalkyl or substituted amino.
R₁ can be alkyl, alkenyl, alkynyl, cycloalkyl, aryl, heterocyclyl, heteroaryl, heterocyclylalkyl, heteroarylalkyl, aralkyl or —(CH₂)ₙO(C═O)NHRₓ,
   wherein n can be an integer 2-10, and
   Rₓ can be alkyl, alkenyl, alkynyl, cycloalkyl, aryl, aralkyl, heteroaryl, heterocyclyl, heterocyclylalkyl or heteroarylalkyl; and
R₂ and R₃ together can form a five-membered acetal, wherein the carbon joining the two oxygen atoms can be substituted with Rₗ and Rₘ,
   wherein Rₗ and Rₘ can be hydrogen, alkyl, alkenyl, alkynyl, cycloalkyl, aryl or aralkyl; Rₗ and Rₘ together can join to form a cyclic ring; or Rₗ and Rₘ together can join to form an oxo,
      wherein the ring optionally can contain one or more heteroatoms selected from O, N or S, and the ring optionally can be substituted with one or more of alkyl, alkenyl, alkynyl, acyl, substituted amino, cycloalkyl, carboxy, oxo, hydroxy, alkoxy, aryloxy, halogen, aryl, aralkyl, heteroaryl, heterocyclyl, heteroarylalkyl, heterocyclylalkyl, or —C(═O)QR₇,
         wherein Q can be O or NH, and R₇ can be alkyl, alkenyl, alkynyl, aryl, aralkyl, cycloalkyl, or heteroarylalkyl; or when Q is NH, R₇ also can be heteroaryl, heterocyclyl or heterocyclylalkyl; or
R₂ and R₃, instead of forming an acetal, optionally and independently can be lower (C₁-C₄)-alkyl, (CH₂)ₖ-aryl, —C(═R_{y})NHRₓ or acyl,
   wherein k can be an integer from 1-4,
   R_{y} can be O or S, and
   Rₓ can be alkyl, alkenyl, alkynyl, cycloalkyl, aryl, aralkyl, heteroaryl, heterocyclyl, heterocyclylalkyl or heteroarylalkyl.

In one embodiment, the Grignard reagent can be an alkyl magnesium halide. In another embodiment, the reaction of the compound of Formula XXX with the compound of Formula XXVIII can be carried out in the presence of a base, for example, triethylamine, diisopropylethylamine, pyridine or a mixture thereof.

The present invention also encompasses processes for preparing compounds of Formula XXXIII comprising reacting a compound of Formula XXXII

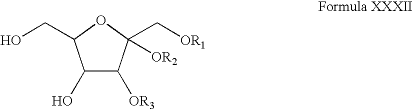

with a compound of Formula XXVIII

R_{f}—N═C═O Formula XXVIII

to form a compound of Formula XXXIII;

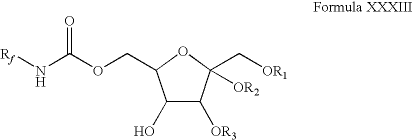

wherein
R_{f} can be hydrogen, alkyl, alkenyl, alkynyl, cycloalkyl, aryl, aralkyl, heterocyclyl, heteroaryl, heteroarylalkyl, heterocyclylalkyl or S(O)₂R₆, wherein R₆ can be alkyl, alkenyl, alkynyl, cycloalkyl, aryl, aralkyl, heteroaryl, heterocyclyl, heterocyclylalkyl, heteroarylalkyl or substituted amino;
R₁ can be alkyl, alkenyl, alkynyl, cycloalkyl, aryl, heterocyclyl, heteroaryl, heterocyclylalkyl, heteroarylalkyl, aralkyl or —(CH₂)ₙO(C═O)NHRₓ,
   wherein n can be an integer 2-10, and
   Rₓ can be alkyl, alkenyl, alkynyl, cycloalkyl, aryl, aralkyl, heteroaryl, heterocyclyl, heterocyclylalkyl or heteroarylalkyl;
      and
R₂ and R₃ together can form a five-membered acetal, wherein the carbon joining the two oxygen atoms can be substituted with Rₗ and Rₘ,
   wherein Rₗ and Rₘ can be hydrogen, alkyl, alkenyl, alkynyl, cycloalkyl, aryl or aralkyl; Rₗ and Rₘ together can join to form a cyclic ring; or Rₗ and Rₘ together can join to form an oxo,
      wherein the ring optionally can contain one or more heteroatoms selected from O, N or S, and the ring optionally can be substituted with one or more of alkyl, alkenyl, alkynyl, acyl, substituted amino, cycloalkyl, carboxy, oxo, hydroxy, alkoxy, aryloxy, halogen, aryl, aralkyl, heteroaryl, heterocyclyl, heteroarylalkyl, heterocyclylalkyl, or —C(═O)QR₇,
         wherein Q can be O or NH, and R₇ can be alkyl, alkenyl, alkynyl, aryl, aralkyl, cycloalkyl, or heteroarylalkyl; or when Q is NH, R₇ also can be heteroaryl, heterocyclyl or heterocyclylalkyl; or
R₂ and R₃, instead of forming an acetal, optionally and independently can be lower (C₁-C₄)-alkyl, (CH₂)ₖ-aryl, —C(═R_{y})NHRₓ or acyl,
   wherein k can be an integer. from 1-4,
   R_{y} can be O or S, and
   Rₓ can be alkyl, alkenyl, alkynyl, cycloalkyl, aryl, aralkyl, heteroaryl, heterocyclyl, heterocyclylalkyl or heteroarylalkyl.

In one embodiment, the reaction can be carried out in the presence of a base, for example, triethylamine, diisopropylethylamine, pyridine or a mixture thereof.

### DETAILED DESCRIPTION OF THE INVENTION

The present invention provides monosaccharides derivatives, which can be used for the inhibition and prevention of cell adhesion and cell adhesion mediated pathologies, including, for example, inflammatory and autoimmune diseases, for example, bronchial asthma, rheumatoid arthritis, type I diabetes, multiple sclerosis, allograft rejection or psoriasis.

Pharmaceutically acceptable salts, pharmaceutically acceptable solvates, enantiomers, diastereomers or N-oxides of these compounds having the same type of activity also are provided.

The present invention also provides for pharmaceutical compositions containing the monosaccharide derivatives of the present invention, which also may contain pharmaceutically acceptable carriers or diluents. Such pharmaceutical compositions can be used for the treatment of inflammatory and autoimmune diseases, for example, bronchial asthma, rheumatoid arthritis, type I diabetes, multiple sclerosis, allograft rejection or psoriasis.

While the present invention has been described in terms of its specific embodiments, certain modifications and equivalents will be apparent to those skilled in the art and are included within the scope of the present invention.

In accordance with one aspect, there is provided compounds having a structure of Formula I,

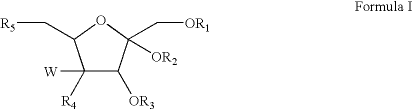

wherein
W can be hydrogen or alkyl;
R₁ can be alkyl, alkenyl, alkynyl, cycloalkyl, aryl, heterocyclyl, heteroaryl, heterocyclylalkyl, heteroarylalkyl, aralkyl or —(CH₂)ₙO(C═O)NHRₓ,
wherein n can be an integer 2-10, and
Rₓ can be alkyl, alkenyl, alkynyl, cycloalkyl, aryl, aralkyl, heteroaryl, heterocyclyl, heterocyclylalkyl or heteroarylalkyl;
   R₂ and R₃ together can form a five membered acetal wherein the carbon joining the two oxygen atoms is substituted with Rₗ and Rₘ,
wherein Rₗ and Rₘ can be hydrogen, alkyl, alkenyl, alkynyl, cycloalkyl, aryl or aralkyl; Rₗ and Rₘ together can join to form a cyclic ring (e.g., a (3-8)-membered cyclic ring); or Rₗ and Rₘ together can join to form an oxo,
   wherein the ring optionally can contain one or more heteroatoms selected from O, N or S, and the ring optionally can be substituted with one or more of alkyl, alkenyl, alkynyl, acyl, substituted amino, cycloalkyl, carboxy, oxo, hydroxy, alkoxy, aryloxy, halogen (e.g., F, Cl, Br or I), aryl, aralkyl, heteroaryl, heterocyclyl, heteroarylalkyl, heterocyclylalkyl, or —C(═O)QR₇,
      wherein Q can be O or NH, and R₇ can be alkyl, alkenyl, alkynyl, aryl, aralkyl, cycloalkyl, or heteroarylalkyl; or when Q is NH, R₇ also can be heteroaryl, heterocyclyl or heterocyclylalkyl;
         R₂ and R₃, instead of forming an acetal, optionally and independently can be lower (C₁-C₄)-alkyl, (CH₂)ₖ-aryl, —C(═R_{y})NHRₓ or acyl,
wherein k can be an integer from 1-4,
R_{y} can be O or S, and
Rₓ can be alkyl, alkenyl, alkynyl, cycloalkyl, aryl, aralkyl, heteroaryl, heterocyclyl, heterocyclylalkyl or heteroarylalkyl; and
   R₅ can be hydrogen, alkyl, cycloalkyl, heteroaryl, heterocyclyl, —NRₚRⱼ, or OR_{z}; or when R₄ is OH, OR_{c} or H, then R₅ can be —NHC(═O)ORₛ, —NHYR_{d}, —NHC(=T)NRₜRₓ or —(CH₂)_{w}(C═O)NRₐR_{b}, wherein
Rₚ and Rⱼ independently can be hydrogen, alkyl, cycloalkyl, aryl, heteroaryl, heterocyclyl, heteroarylalkyl, heterocyclylalkyl, or aralkyl, or Rₚ and Rⱼ may together join to form a cyclic ring (5-8 membered), which optionally may be benzofused, containing 0-4 heteroatom selected from 0-4 heteroatoms selected from O, S, or N wherein the ring may be substituted with one or more of alkyl, alkenyl, alkynyl, amino, substituted amino, cycloalkyl, carboxy, oxo, hydroxy, alkoxy, aryloxy, halogen, aryl, aralkyl, heteroaryl, heterocyclyl, heteroarylalkyl or heterocyclylalkyl;
wherein R_{z} can be alkyl, alkenyl, alkynyl, cycloalkyl, aryl, aralkyl, heteroaryl, heterocyclyl, heteroarylalkyl, heterocyclylalkyl, acyl or —C(═O)NR_{f}R_{q},
   wherein R_{f} and R_{q} independently can be hydrogen, alkyl, alkenyl, alkynyl, cycloalkyl, aryl, aralkyl, heterocyclyl, heteroaryl, heteroarylalkyl, heterocyclylalkyl or S(O)₂R₆; or R_{f} and R_{q} can together form a ring,
      wherein R₆ can be alkyl, alkenyl, alkynyl, cycloalkyl, aryl, aralkyl, heteroaryl, heterocyclyl, heterocyclylalkyl, heteroarylalkyl or substituted amino;
Rₛ can be alkyl, alkenyl, alkynyl, cycloalkyl, aryl, aralkyl, heterocyclylalkyl or heteroarylalkyl,
Y can be —C(═O), —C(═S) or SO₂),
R_{d} can be alkyl, alkenyl, alkynyl, cycloalkyl, aryl, aralkyl, heteroaryl, heterocyclyl, heteroarylalkyl or heterocyclylalkyl,
T can be O, S, —N(CN), —N(NO₂), or —CH(NO₂),
Rₜ can be H, OH or Rₓ,
Rₓ can be alkyl, alkenyl, alkynyl, cycloalkyl, aryl, aralkyl, heteroaryl, heterocyclyl, heterocyclylalkyl or heteroarylalkyl,
w can be 1-4, and
Rₐ and R_{b} independently can be hydrogen or R_{d}, or Rₐ and R_{b}, together with the nitrogen atom carrying them, can be the N-terminus of an amino acid or di-tetrapeptide,
   wherein R_{f} and R_{q} independently can be hydrogen, alkyl, alkenyl, alkynyl, cycloalkyl, aryl, aralkyl, heterocyclyl, heteroaryl, heteroarylalkyl, heterocyclylalkyl or S(O)₂R₆; or R_{f} and R_{q} can together form a ring,
      wherein R₆ can be alkyl, alkenyl, alkynyl, cycloalkyl, aryl, aralkyl, heteroaryl, heterocyclyl, heterocyclylalkyl, heteroarylalkyl or substituted amino;
         and wherein when W is hydrogen, then R₄ can be hydrogen, OR_{c}, —NHC(═O)ORₛ, —NHYR_{d}, —NHC(=T)NRₜRₓ, or —(CH₂)_{w}(C═O)NRₐR_{b},
wherein R_{c} can be hydrogen, alkyl, cycloalkyl, aryl, heteroaryl, heterocyclyl, acyl or —C(═O)NR_{f}R_{q},
Rₛ can be alkyl, alkenyl, alkynyl, cycloalkyl, aryl, aralkyl, heterocyclylalkyl or heteroarylalkyl,
Y can be —C(═O), —C(═S) or SO₂),
R_{d} can be alkyl, alkenyl, alkynyl, cycloalkyl, aryl, aralkyl, heteroaryl, heterocyclyl, heteroarylalkyl or heterocyclylalkyl,
T can be O, S, —N(CN), —N(NO₂), or —CH(NO₂),
Rₜ can be H, OH or Rₓ,
Rₓ can be alkyl, alkenyl, alkynyl, cycloalkyl, aryl, aralkyl, heteroaryl, heterocyclyl, heterocyclylalkyl or heteroarylalkyl,
w can be 1-4, and
Rₐ and R_{b} independently can be hydrogen or R_{d}, or Rₐ and R_{b}, together with the nitrogen atom carrying them, can be the N-terminus of an amino acid or di-tetrapeptide,
   wherein R_{f} and R_{q} independently can be hydrogen, alkyl, alkenyl, alkynyl, cycloalkyl, aryl, aralkyl, heterocyclyl, heteroaryl, heteroarylalkyl, heterocyclylalkyl or S(O)₂R₆; or R_{f} and R_{q} can together form a ring,
      wherein R₆ can be alkyl, alkenyl, alkynyl, cycloalkyl, aryl, aralkyl, heteroaryl, heterocyclyl, heterocyclylalkyl, heteroarylalkyl or substituted amino; or
         when W is alkyl, then R₄ can be —OR_{z},
wherein R_{z} can be alkyl, alkenyl, alkynyl, cycloalkyl, aryl, aralkyl, heteroaryl, heterocyclyl, heteroarylalkyl, heterocyclylalkyl, acyl or —C(═O)NR_{f}R_{q},
   wherein R_{f} and R_{q} independently can be hydrogen, alkyl, alkenyl, alkynyl, cycloalkyl, aryl, aralkyl, heterocyclyl, heteroaryl, heteroarylalkyl, heterocyclylalkyl or S(O)₂R₆; or R_{f} and R_{q} can together form a ring,
      wherein R₆ can be alkyl, alkenyl, alkynyl, cycloalkyl, aryl, aralkyl, heteroaryl, heterocyclyl, heterocyclylalkyl, heteroarylalkyl or substituted amino.

In one embodiment, when R₅ is OR_{z} and R₄ is OR_{c}, then R_{c} and R_{z} can be joined together to form a six-membered acetal, wherein the carbon joining the oxygens is substituted with Rₗ and Rₘ wherein Rₗ and Rₘ can be hydrogen, alkyl, alkenyl, alkynyl, cycloalkyl, aryl or aralkyl; Rₗ and Rₘ together can join to form a cyclic ring (e.g., a (3-8)-membered cyclic ring); or Rₗ and Rₘ together can join to form an oxo,
wherein the ring optionally can contain one or more heteroatoms selected from O, N or S, and the ring optionally can be substituted with one or more of alkyl, alkenyl, alkynyl, acyl, substituted amino, cycloalkyl, carboxy, oxo, hydroxy, alkoxy, aryloxy, halogen (e.g., F, Cl, Br or I), aryl, aralkyl, heteroaryl, heterocyclyl, heteroarylalkyl, heterocyclylalkyl, —C(═O)QR₇,
   wherein Q can be O or NH, and R₇ can be alkyl, alkenyl, alkynyl, aryl, aralkyl, cycloalkyl, or heteroarylalkyl; or when Q is NH, R₇ also can be heteroaryl, heterocyclyl or heterocyclylalkyl.

The following definitions apply to terms as used herein.

The term “alkyl,” unless otherwise specified, refers to a monoradical branched or unbranched saturated hydrocarbon chain having from 1 to 20 carbon atoms. This term can be exemplified by groups, for example, methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, aryl, heterocyclyl, heteroaryl, arylthio, thiol, alkylthio, aryloxy, nitro, aminosulfonyl, aminocarbonylamino, —NHC(═O)Rₓ, —NRₐR_{b}, —C(═O)NRₐR_{b}, —NHC(═O)NRₓRₜ, —C(═O)heteroaryl, C(═O)heterocyclyl, —O—C(═O)NRₐR_{b} wherein Rₓ, Rₜ, Rₐ and R_{b} are the same as defined earlier, nitro, —S(O)ₘR₆ (wherein m is an integer from 0-2 and R₆ can be alkyl, alkenyl, alkynyl, cycloalkyl, aryl, aralkyl, heteroaryl, heterocyclyl, heterocyclylalkyl, heteroarylalkyl or substituted amino). Unless otherwise constrained by the definition, alkyl substituents may be further substituted by 1-3 substituents selected from alkyl, carboxy, —NRₐR_{b}, —C(═O)NRₐR_{b}, —OC(═O)NRₐR_{b}, —NHC(═O)NRₐR_{b} (wherein Rₐ and R_{b} are the same as defined earlier), hydroxy, alkoxy, halogen, CF₃, cyano, and —S(O)ₘR₆, (where R₆ and m are the same as defined earlier); or an alkyl group as defined above may also be interrupted by 1-5 atoms of groups independently chosen from oxygen, sulfur and —NRₐ—, where Rₐ is chosen from hydrogen, alkyl, cycloalkyl, alkenyl, cycloalkenyl, alkynyl, aryl, acyl, aralkyl, —C(═O)ORₛ wherein Rₛ is the same as defined earlier, S(O)₂R₆ (where R₆ is as defined earlier), —C(═O)NRₐR_{b} (wherein Rₐ and R_{b} are as defined earlier). Unless otherwise constrained by the definition, all substituents may be further substituted by 1-3 substituents chosen from alkyl, carboxy, —NRₐR_{b}, —C(═O)NRₐR_{b}, —O—C(═O)NRₐR_{b} wherein Rₐ and R_{b} are the same as defined earlier hydroxy, alkoxy, halogen, CF₃, cyano, and —S(O)ₘR₆, where m and R₆ are the same as defined earlier; or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group preferably having from 2 to 20 carbon atoms with cis or trans geometry. In the event that alkenyl is attached to the heteroatom, the double bond cannot be alpha to the heteroatom. Alkenyl groups may further be substituted with one or more substituents selected from alkyl, alkynyl, alkoxy, cycloalkyl, cycloalkenyl, acyl, acylamino, acyloxy, —NHC(═O)Rₓ, —NRₐR_{b}, —C(═O)NRₐR_{b}, —NHC(═O)NRₓRₜ, —O—C(═O)NRₐR_{b} (wherein Rₐ and R_{b} are the same as defined earlier), alkoxycarbonylamino, azido, cyano, halogen, hydroxy, oxo, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, nitro, or S(O)ₘR₆ (wherein R₆ and m are the same as defined earlier). Unless otherwise constrained by the definition, all substituents may optionally be further substituted by 1-3 substituents selected from alkyl, carboxy, hydroxy, alkoxy, halogen, —CF₃, cyano, —NRₐR_{b}, —C(═O)NRₐR_{b}, —O—C(═O)NRₐR_{b} (wherein Rₐ and R_{b} are the same as defined earlier) or —S(O)ₘR₆ (wherein R₆ and m are the same as defined earlier).

The term “alkynyl,” unless specified refers to a monoradical of an unsaturated hydrocarbon, preferably having from 2 to 20 carbon atoms. In the event that alkynyl is attached to the heteroatom, the triple bond cannot be alpha to the heteroatom. Alkynyl groups may further be substituted with one or more substituents selected from alkyl, alkenyl, alkoxy, cycloalkyl, cycloalkenyl, acyl, acylamino, acyloxy, alkoxycarbonylamino, azido, cyano, halogen, hydroxy, oxo, thiocarbonyl, carboxy, arylthio, thiol, alkylthio, aryl, aralkyl, aryloxy, aminosulfonyl, aminocarbonylamino, nitro, heterocyclyl, heteroaryl, heterocyclylalkyl, heteroarylalkyl, —NHC(═O)Rₓ, —NRₐR_{b}, —NHC(═O)NRₓRₜ, —C(═O)NRₐR_{b}, —O—C(═O)NRₐR_{b} (wherein Rₓ, Rₜ, Rₐ and R_{b} are the same as defined earlier), —S(O)ₘR₆ (wherein R₆ and m are the same as defined earlier). Unless otherwise constrained by the definition, all substituents may optionally be further substituted by 1-3 substituents chosen from alkyl, carboxy, carboxyalkyl, hydroxy, alkoxy, halogen, CF₃, —NRₐR_{b}, —C(═O)NRₐR_{b}, —NHC(═O)NRₓRₜ, —C(═O)NRₐR_{b} (wherein Rₓ, Rₜ, Rₐ and R_{b} are the same as defined earlier), cyano, and —S(O)ₘR₆ (where R₆ and m are the same as defined earlier).

The term “cycloalkyl,” unless otherwise specified, refers to cyclic alkyl groups of from 3 to 20 carbon atoms having a single cyclic ring or multiple condensed rings, which optionally may contain one or more olefinic bonds. Such cycloalkyl groups include, by way of example, single ring structures, for example, cyclopropyl, cyclobutyl, cyclooctyl, cyclopentenyl, and the like, or multiple ring structures, for example, adamantanyl, and bicyclo [2.2.1]heptane, or cyclic alkyl groups to which is fused an aryl group, for example, indane, and the like. Cycloalkyl groups may further be substituted with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NRₐR_{b}, —NHC(═O)NRₓRₜ, —NHC(═O)Rₓ, —C(═O)NRₐR_{b}, —O—C(═O)NRₐR_{b} (wherein Rₓ, Rₜ, Rₐ and R_{b} are the same as defined earlier), nitro, heterocyclyl, heteroaryl, heterocyclylalkyl, heteroarylalkyl, S(O)ₘ—R₆ (wherein R₆ and m are the same as defined earlier). Unless otherwise constrained by the definition, all substituents may optionally be further substituted by 1-3 substituents chosen from alkyl, carboxy, hydroxy, alkoxy, halogen, CF₃, —NRₐR_{b}, —C(═O)NRₐR_{b}, —NHC(═O)NRₓRₜ, —O—C(═O)NRₐR_{b} (wherein Rₓ, R_{y}, Rₐ, and R_{b} are the same as defined earlier), cyano, and —S(O)ₘR₆ (wherein R₆ and m are the same as defined earlier).

The term “alkoxy,” unless otherwise specified, refers to the group O-alkyl, wherein alkyl is the same as defined above.

The term “aralkyl,” unless otherwise specified, refers to alkyl-aryl linked through alkyl (wherein alkyl is the same as defined above) portion and the alkyl portion contains carbon atoms from 1-6 and aryl is as defined below. The examples of aralkyl groups include benzyl, ethylphenyl and the like.

The term “aryl,” unless otherwise specified, refers to a carbocyclic aromatic group, for example phenyl, anthryl, biphenyl or naphthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryl, aryloxy, cyano, nitro, CF₃, OCF₃, COORₑ (wherein Rₑ can be hydrogen, alkyl, alkenyl, cycloalkyl, aralkyl, heterocyclylalkyl, heteroarylalkyl), NHC(═O)Rₓ, —NRₐR_{b}, —C(═O)NRₐR_{b}, —NHC(═O)NRₓRₜ, —O—C(═O)NRₐR_{b} (wherein Rₐ and R_{b} are the same as defined earlier), —(SO₂)ₘR₆ (wherein R₆ and m are the same as defined earlier), carboxy, heterocyclyl, heteroaryl, heterocyclylalkyl, heteroarylalkyl or amino carbonyl amino. The aryl group may optionally be fused with cycloalkyl group, wherein the cycloalkyl group may optionally contain heteroatoms selected from O, N or S.

The term “aryloxy” denotes the group O-aryl wherein aryl is the same as defined above.

The term “carboxy” as defined herein refers to —C(═O)OH.

The term “heteroaryl,” unless otherwise specified, refers to an aromatic ring structure containing 5 or 6 carbon atoms, or a bicyclic aromatic group having 8 to 10 carbon atoms, with one or more heteroatom(s) independently selected from N, O or S, optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, —NRₐR_{b}, —(CH₂)_{w}C(═O)R_{g} (wherein w is an integer from 1-4 and R₉ is hydroxy, OR_{z}, NRₐR_{b}, —NHOR_{z} or —NHOH), —C(═O)NRₐR_{b}, —NHC(═O)NRₓRₜ, —S(O)ₘR₆, or —O—C(═O)NRₐR_{b} (wherein m, R₆, R_{z}, Rₜ, Rₓ, Rₐ and R_{b} are the same as defined earlier). Unless or otherwise constrained by the definition, of the substituents are attached to the ring atom, be it carbon or heteroatom. Examples of heteroaryl groups are pyridinyl, pyridazinyl, pyrimidinyl, pyrrolyl, oxazolyl, thiazolyl, thienyl, isoxazolyl, triazinyl, furanyl, benzofuranyl, indolyl, benzothiazolyl, benzoxazolyl, and the like.

The term “heterocyclyl,” unless otherwise specified, refers to a non aromatic monocyclic or bicyclic cycloalkyl group having 5 to 10 atoms in which 1 to 4 carbon atoms in a ring are replaced by heteroatoms selected from O, S or N, and are optionally benzofused or fused heteroaryl of 5-6 ring members and/or are optionally substituted with one or more of halogen (e.g., F, Cl, Br, I), hydroxy, alkyl, alkenyl, alkynyl, cycloalkyl, acyl, aryl, alkoxy, alkaryl, cyano, nitro, oxo, carboxy, —C(═O)NRₐR_{b}, SO₂R₆, —O—C(═O)NRₐR_{b}, —NHC(═O)NRₓRₜ, or —NRₐR_{b} (wherein R₆, Rₓ, Rₜ, Rₐ and R_{b} are the same as defined earlier). Unless or otherwise constrained by the definition, the substituents are attached to the ring atom, be it carbon or heteroatom. Also unless or otherwise constrained by the definition the heterocyclyl ring may optionally contain one or more olefinic bond(s). Examples of heterocyclyl groups include oxazolidinyl, tetrahydrofuranyl, dihydrofuranyl, benzoxazinyl, benzthiazinyl, benzimidazolyl, carbaxolyl, indolyl, phenoxazinyl, phenothiazinyl, dihydropyridinyl, dihydroisoxazolyl, dihydrobenzofuryl, azabicyclohexyl, dihydroindolyl, pyridinyl, isoindole 1,3-dione, piperidinyl or piperazinyl.

“Heteroarylalkyl,” unless otherwise specified, refers to alkyl-heteroaryl group linked through alkyl portion, wherein the alkyl and heteroaryl are the same as defined earlier.

“Heterocyclylalkyl,” unless otherwise specified, refers to alkyl-heterocyclyl group linked through alkyl portion, wherein the alkyl and heterocyclyl are the same as defined earlier.

“Acyl,” unless otherwise specified, refers to —C(═O)R″ wherein R″ is selected from alkyl, cycloalkyl, aryl, aralkyl, heteroaryl, heterocyclyl, heteroarylalkyl or heterocyclylalkyl.

“Substituted amino,” unless otherwise specified, refers to a group —N(Rₖ)₂, wherein each Rₖ is independently selected from hydrogen (provided that both Rₖ groups are not hydrogen (defined as “amino”)), alkyl, alkenyl, alkynyl, aralkyl, cycloalkyl, aryl, heteroaryl, heterocyclyl, heterocyclylalkyl, heteroarylalkyl, acyl, S(O)ₘR₆ (wherein m and R₆ is the same as defined above), —C(═R_{y})NRₐR_{b} (wherein R_{y}, Rₐ and R_{b} are the same as defined earlier) or NHC(═R_{y})NRₜRₓ (wherein R_{y}, Rₜ and Rₓ are the same as defined earlier).

Unless otherwise constrained by the definition, all substituents may optionally be further substituted by 1-3 substituents chosen from alkyl, aralkyl, cycloalkyl, aryl, heteroaryl, heterocyclyl, carboxy, carboxyalkyl, hydroxy, alkoxy, halogen, CF₃, cyano, —C(═R_{y})NRₐR_{b}, —O(C═O)NRₐR_{b} (wherein Rₐ, R_{b} and R_{y} are the same as defined earlier) and —OC(═R_{y})NRₐR_{b}, —S(O)ₘR₆ (where R₆ is the same as defined above and m is 0-2).

The term “leaving group,” unless otherwise specified, generally refers to groups that exhibit the desirable properties of being labile under the defined synthetic conditions and also, of being easily separated from synthetic products under defined conditions. Examples of such leaving groups include, but are not limited to, halogen (e.g., F, Cl, Br, I), triflates, tosylate, mesylates, alkoxy, thioalkoxy, hydroxy radicals and the like.

The term “activated derivative of a carboxylic acid,” for example, that of a suitable protected amino acid, aliphatic acid or an aromatic acid, refer to the corresponding acyl halide (e.g., acid fluoride, acid chloride or acid bromide), corresponding activated esters (e.g., nitro phenyl ester, the ester of 1-hydroxybenzotriazole or the ester of hydroxysuccinimide, HOSu) or a mixed anhydride for example anhydride with ethyl chloroformate and other conventional derivatives within the skill of the art.

The term “protecting groups,” unless otherwise specified, refers to moieties that prevent chemical reaction at a location of a molecule intended to be left unaffected during chemical modification of such molecule. Unless otherwise specified, protecting groups may be used on groups, such as hydroxy, amino, or carboxy. Examples of protecting groups are found in T. W. Greene and P. G. M. Wuts, “Protective Groups in Organic Synthesis”, 2^{nd} Ed., John Wiley and Sons, New York, N.Y., which is incorporated herein by reference. The species of the carboxylic protecting groups, amino protecting groups or hydroxy protecting groups employed are not critical, as long as the derivatised moieties/moiety is/are stable to conditions of subsequent reactions and can be removed without disrupting the remainder of the molecule.

The terms “pharmaceutically acceptable salts” or “pharmacologically acceptable salts,” unless otherwise specified, refer to derivatives of compounds that can be modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like.

The term “amino acid,” unless otherwise specified, refers to both natural and unnatural amino acids. The term “natural amino acid,” unless otherwise specified, refers to the twenty two naturally occurring amino acids glycine, alanine, valine, leucine, isoleucine, serine, methionine, threonine, phenylalanine, tyrosine, trytophan, cysteine, proline, proline, histidine, aspartic acid, asparagines, glutamic acid, glutamine, γ-carboxyglutamic acid, arginine, ornithine and lysine in their L form. The term “unnatural amino acid,” unless otherwise specified, refers to the ‘D’ form of the twenty two naturally occurring amino acids described above. It is further understood that the term unnatural amino acid includes homologues of the natural amino acids, and synthetically modified form of the natural amino acids commonly utilized by those in the peptide chemistry arts when preparing synthetic analogues of naturally occurring peptides, including D and L forms. The synthetically modified forms include amino acids having alkylene chains shortened or lengthened by up to two carbon atoms, amino acids comprising optionally substituted aryl groups, and amino acids comprised halogenated groups preferably halogenated alkyl and aryl groups.

The term “unnatural amino acids,” unless otherwise specified, also refers to beta amino acids.

The term “peptide,” unless otherwise specified, refers to a molecule comprising a series of amino acids linked through amide linkages. A dipeptide refers to a peptide having 2 amino acids, a tripeptide refers to a peptide having 3 amino acids and tetrapeptide refers to a peptide having four amino acids, wherein the term amino acid is as defined earlier.

The compounds of this invention contain one or more asymmetric carbon atoms and thus, can exist as racemates and racemic mixtures, single enantiomers, diastereomeric mixtures or individual diastereomers. All such isomeric forms of these compounds are expressly encompassed by the present invention. Each stereogenic carbon can have an R or S configuration. Although the specific compounds exemplified in this application may be depicted in a particular stereochemical configuration, compounds having either the opposite stereochemistry at each chiral center, or mixtures thereof, are contemplated in the invention. Although amino acids and amino acid side chains may be depicted in a particular configuration, both natural and unnatural forms are contemplated in the invention. Also, geometric isomers of olefins, C═N double bonds and the like, can be present in the compounds of this invention, and all such stable isomers are contemplated in the present invention.

The compounds of the present invention can be prepared by techniques well known in the art and familiar to skilled synthetic organic chemist. In addition, the compounds of the present invention can be prepared, for example, by following the reaction schemes as depicted.

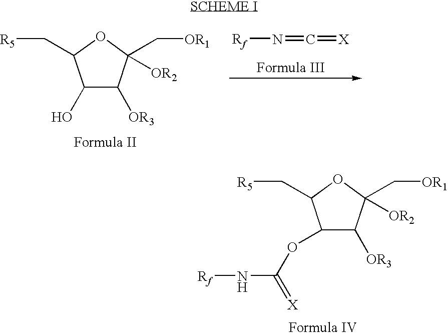

A compound of Formula IV can be prepared following Scheme I. Accordingly, a compound of Formula II (wherein R₁, R₂, R₃ and R₅ are as described earlier) reacts with a compound of Formula III (wherein X is O, S and R_{f} is same as described earlier) to form a compound of Formula IV. This reaction can be carried out in an organic solvent, for example, dichloromethane, dichloroethane, chloroform or carbon tetrachloride.

Compounds prepared using Scheme I include, for example:
1-O-decyl-2,3-O-isopropylidene-4-O-{[(phenyl-sulfonyl)-amino]-carbonyl}-6-deoxy-α-L-sorbofuranoside (Compound No. 1);
1-O-Dodecyl-2,3-O-isopropylidene-4-O-{[(4-chloro-phenyl)-sulfonylamino]-carbonyl}-6-deoxy-6-(1-azepanyl)-α-L-sorbofuranoside (Compound No. 2);
1-O-Dodecyl-2,3-O-isopropylidene-4-O-{[phenyl-sulfonylamino]-carbonyl}-6-deoxy-6-(1-azepanyl)-α-L-sorbofuranoside (Compound No. 3);
1-O-Dodecyl-2,3-O-isopropylidene-4-O-{[(4-methyl-phenyl)-sulfonylamino]-carbonyl}-6-deoxy-6-(1-azepanyl)-α-L-sorbofuranoside (Compound No. 4);
1-O-Dodecyl-2,3-O-isopropylidene-4-O-{[(4-methyl-phenyl)-sulfonylamino]-carbonyl}-6-deoxy-6-(4-morpholinyl)-α-L-sorbofuranoside (Compound No. 5);
1-O-Dodecyl-2,3-O-isopropylidene-4-O-{[(4-chloro-phenyl)-sulfonylamino]-carbonyl}-6-deoxy-6-(4-morpholinyl)-α-L-sorbofuranoside (Compound No. 6);
1-O-Dodecyl-2,3-O-isopropylidene-4-O-{[(phenyl-sulfonyl)-amino]-carbonyl}-6-deoxy-6-(4-morpholinyl)-α-L-sorbofuranoside (Compound No. 7);
1-O-Dodecyl-2,3-O-isopropylidene-4-O-{[(phenylsulfonyl)-amino]-carbonyl}-6-deoxy-6-(1-pyrrolidinyl)-α-L-sorbofuranoside (Compound No. 8);
1-O-Dodecyl-2,3-O-isopropylidene-4-O-{[(4-methyl-phenyl)-sulfonylamino]-carbonyl}-6-deoxy-α-L-sorbofuranoside (Compound No. 9);
Hydrochloride salt of 1-O-Dodecyl-2,3-O-isopropylidene-4-O-{[(4-methyl-phenyl)-sulfonylamino]-carbonyl}-6-deoxy-6-(1-pyrrolidinyl)-α-L-sorbofuranoside (Compound No. 10);
Hydrochloride salt of 1-O-Dodecyl-2,3-O-isopropylidene-4-O-{[(4-chloro-phenyl)-sulfonylamino]-carbonyl}-6-deoxy-6-(1-pyrrolidinyl)-α-L-sorbofuranoside (Compound No. 11);
1-O-Heptyl-2,3-O-isopropylidene-4-O-{[4-(2-methoxy-2-oxo-ethyl)-phenyl]-amino}-carbonyl-6-deoxy-6-(1-azepanyl)-α-L-sorbofuranoside (Compound No. 12);
1-O-Heptyl-2,3-O-isopropylidene-4-O-{[4-(2-methoxy-2-oxo-ethyl)-phenyl]-amino}-carbonyl-6-deoxy-6-(4-morpholinyl)-α-L-sorbofuranoside (Compound No. 13);
Hydrochloride salt of 1-O-heptyl-2,3-O-isopropylidene-4-O-{[4-(2-methoxy-2-oxo-ethyl)-phenyl]-amino}-carbonyl-6-deoxy-6-(1-azepanyl)-α-L-sorbofuranoside (Compound No. 14);
1-O-Dodecyl-2,3-O-isopropylidene-4-O-{[4-(2-methoxy-2-oxo-ethyl)-phenyl]-amino}-carbonyl-6-deoxy-6-(1-azepanyl)-α-L-sorbofuranoside (Compound No. 15);
1-O-Dodecyl-2,3-O-isopropylidene-4-O-{[4-(2-methoxy-2-oxo-ethyl)-phenyl]-amino}-carbonyl-6-deoxy-6-(4-morpholinyl)-α-L-sorbofuranoside (Compound No. 16);
1-O-Dodecyl-2,3-O-isopropylidene-4-O-{[4-(2-methoxy-2-oxo-ethyl)-phenyl]-amino}-carbonyl-6-deoxy-6-(1-pyrrolidinyl)-α-L-sorbofuranoside (Compound No. 17);
1-O-Dodecyl-2,3-O-isopropylidene-4-O-{[4-(2-methoxy-2-oxo-ethyl)-phenyl]-amino}-carbonyl-6-deoxy-6-(1-piperidinyl)-α-L-sorbofuranoside (Compound No. 18);
1-O-Heptyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 19);
1-O-Decyl-2,3-O-isopropylidene-4-O-{[(4-[2-hydroxy-2-oxo-ethyl]-phenyl)-amino]-carbonyl}-6-deoxy-6-(1-azepanyl)-α-L-sorbofuranoside (Compound No. 20);
Tris salt of 1-O-Heptyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 21);
Tris salt of-1-O-decyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 22);
1-O-Dodecyl-2,3-O-isopropylidene-4-O-{[4-(2-hydroxy-2-oxo-ethyl)-phenyl]-amino}-carbonyl-6-deoxy-6-(1-piperidinyl)-α-L-sorbofuranoside (Compound No. 23);
1-O-Dodecyl-2,3-O-isopropylidene-4-O-{[4-(2-hydroxy-2-oxo-ethyl)-phenyl]-amino}-carbonyl-6-deoxy-6-(4-morpholinyl)-α-L-sorbofuranoside (Compound No. 24);
1-O-Dodecyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 25);
1-O-Dodecyl-2,3-O-isopropylidene-4-O-{[4-(2-hydroxy-2-oxo-ethyl)-phenyl]-amino}-carbonyl-6-deoxy-6-(1-pyrrolidinyl)-α-L-sorbofuranoside (Compound No. 26);
1-O-Dodecyl-2,3-O-isopropylidene-4-O-[4-(2-hydroxy-2-oxo-phenyl)-amino]-carbonyl-6-deoxy-6-(1-azepanyl)-α-L-sorbofuranoside (Compound No. 27);
Tris salt of 1-O-Decyl-2,3-O-isopropylidene-4-O-[(4-{2-hydroxy-2-oxo-ethyl}-phenyl)-amino]-carbonyl-6-deoxy-6-[2-(1-pyrrolidinyl)-ethyl]-amino-α-L-sorbofuranoside (Compound No. 28);
Tris salt of 1-O-Decyl-2,3-O-isopropylidene-4-O-[{4-(2-hydroxy-2-oxo-ethyl)-phenyl}-amino]-carbonyl-6-deoxy-6-[2-(1-piperidinyl)-ethyl]amino-α-L-sorbofuranoside. (Compound No. 29);
Tris salt of 1-O-Decyl-2,3-O-isopropylidene-4-O-{[4-(2-hydroxy-2-oxo-ethyl)-phenyl]-amino}-carbonyl-6-deoxy-6-[2-(4-morpholinyl)-ethyl]-amino-α-L-sorbofuranoside (Compound No. 30);
Tris salt of 1-O-Decyl-2,3-O-isopropylidene-4-O-{[4-(2-hydroxy-2-oxo-ethyl)-phenyl]-amino}-carbonyl-6-deoxy-6-[2-(1-cycloheptyl-amino)-ethyl]-amino-α-L-sorbofaranoside (Compound No. 31).

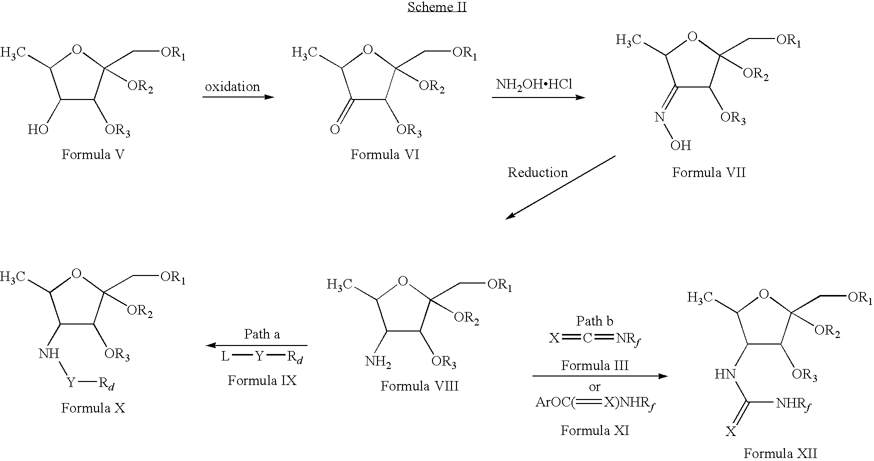

A compound of Formula X and XII can be prepared, for example, following Scheme II. Accordingly, a compound of Formula V (wherein R₁, R₂ and R₃ are same as defined earlier) can oxidize to form a compound of Formula VI. This reaction can be carried out in an organic solvent, for example, dichloromethane, diethyl ether, tetrahydrofuran in the presence of oxidizing agents, for example, pyridinium dichromate; pyridinium chlorochromate; dimethylsulfoxide in combination with acetic anhydride, oxalyl chloride, or trifluoroacetic anhydride; periodinane, or mixtures thereof.

The compound of Formula VI can react with hydroxylamine hydrochloride to form a compound of Formula VII. This reaction can be carried out in an organic solvent, for example, ethanol, methanol, propanol or isopropyl alcohol in the presence of a base, for example pyridine, diisopropylethylamine, triethylamine, or mixtures thereof.

The compound of Formula VII can be reduced to a compound of Formula VIII. This reaction can be carried out in an organic solvent, for example, tetrahydrofuran, dimethylformamide, diethylether, dioxane, or a mixture thereof in the presence of at least one reducing agent, for example, lithium aluminum hydride, sodium borohydride, or a mixture thereof.

The compound of Formula VIII can be reacted via Path a to form a compound of Formula X. Accordingly in Path a, the compound of Formula VIII can be reacted with a compound of Formula IX (wherein R_{d} is same as defined earlier, L is a leaving group, for example, OH (activated in-situ, as known to a skilled practitioner) or halogen (e.g., Cl, Br or I) and Y is C═O or SO₂) to give the compound of Formula X.

The reaction of a compound of Formula VIII with a compound of Formula IX (wherein Y is C(═O) and L is OH) to give a compound of Formula X (Path a) through the intermediacy of an activated derivative of a carboxylic acid can be carried out in an organic solvent, as well as in the presence of at least one condensing agent and/or an base. Examples of the organic solvent include dimethylformamide, dioxane, tetrahydrofuran, or a mixture thereof. Examples of the at least one condensing agent include 1-(3-dimethylaminopropyl)-3-ethyl carbodiimide hydrochloride, dicyclohexylcarbodiimide, or a mixture thereof. Examples of a base include N-methylmorpholine, diisopropylamine, triethylamine, or a mixture thereof. Alternatively, this reaction can be carried out through a mixed anhydride by reacting the compound of Formula IX with a chloroformate, for example, ethyl chloroformate or isobutylchloroformate.

The reaction of a compound of Formula VIII with a compound of Formula IX (wherein Y is C═O or SO₂ and L is Cl) to give a compound of Formula X (Path a) can be carried out in an organic solvent and in the presence of a base. Examples of organic solvents include dichloromethane, dichloroethane, chloroform carbon tetrachloride, tetrahydrofuran, dimethylformamide, or mixtures thereof. Examples of bases include pyridine, triethylamine, diisopropylethylamine or mixtures thereof.

The compound of Formula VIII also can be reacted via Path b to form a compound of Formula XII. Accordingly in Path b, the compound of Formula VIII is reacted with a compound of Formula III or with a compound of Formula XI (wherein Ar is aryl, R_{f} is same as defined earlier) to form a compound of Formula XII.

The reaction of a compound of Formula VIII with a compound of Formula III or a compound of Formula XI to give a compound of Formula XII (Path b) can be carried out in an organic solvent and in the presence of a base. Examples of organic solvents include dichloromethane, dichloroethane, dimethylsulfoxide, tetrahydrofuran, dimethylformamide, or mixtures thereof. Examples of bases include triethylamine, diisopropylethylamine, pyridine, or mixtures thereof.

Compounds prepared using Scheme II include, for example:
(4ξ)-1-O-Dodecyl-2,3-O-isopropylidene-4,6-dideoxy-4-[{[(4-fluoro-phenyl)-amino]-carbonyl}-amino]-α-L-erythro-hex-2-ulofuranoside (Compound No. 32);
(4ξ)-1-O-Dodecyl-2,3-O-isopropylidene-4,6-dideoxy-4-{[(butyl-amino)-carbonyl]-amino}-α-L-erythro-hex-2-ulofuranoside (Compound No. 33);
(4ξ)-1-O-Dodecyl-2,3-O-isopropylidene-4,6-dideoxy-4-{[(4-fluoro-phenyl)-sulfonyl]-amino}-α-L-erythro-hex-2-ulofuranoside (Compound No. 34);
(4ξ)-1-O-Dodecyl-2,3-O-isopropylidene-4,6-dideoxy-4-{[(4-fluoro-phenyl)-carbonyl]-amino}-α-L-erythro-hex-2-ulofuranoside (Compound No. 35);
(4ξ)-1-O-Dodecyl-2,3-O-isopropylidene-4,6-dideoxy-4-[{[(2-phenylethyl)-amino]-thiocarbonyl}-amino]-α-L-erythro-hex-2-ulofuranoside (Compound No. 36);
(4ξ)-1-O-Dodecyl-2,3-O-isopropylidene-4,6-dideoxy-4-[f{[(4-[2-hydroxy-2-oxo-ethyl]-phenyl)-amino]-carbonyl}-amino]-α-L-erythro-hex-2-ulofuranoside (Compound No. 37);
(4ξ)-1-O-Dodecyl-2,3-O-isopropylidene-4,6-dideoxy-4-{[3-(1,3-benzodionol-5-yl)-propanoyl]-amino}-α-L-erythro-hex-2-ulofuranoside (Compound No. 38).

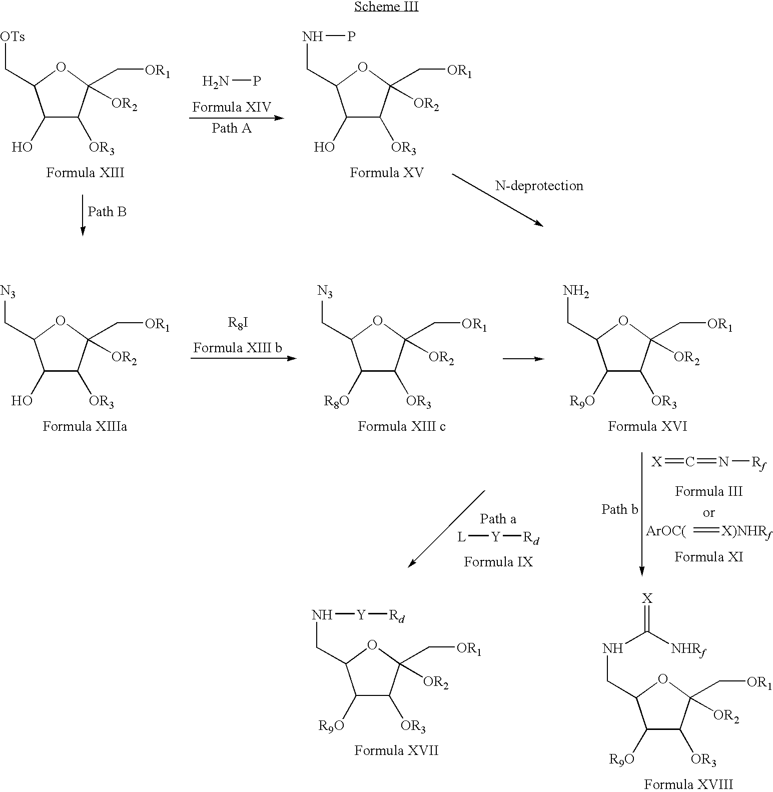

Compounds of Formula XVII and XVIII can be prepared following Scheme III. Accordingly,

Path A: a compound of Formula XIII can be reacted with a compound of Formula XIV (wherein P is a protecting group, for example, aralkyl or acyl) to form a compound of Formula XV.

The compound of Formula XIV can be deprotected to form a compound of Formula XVI under deprotection conditions. This reaction can be carried in an organic solvent, for example, methanol, ethanol, propanol, isopropylalcohol, tetrahydrofuran or ethyl acetate. Examples of deprotection conditions include hydrogenation utilizing palladium on carbon or under catalytic transfer hydrogenation condition of ammonium formate and palladium on carbon. Alternatively, the tosylate can be displaced with an azido group, reduction of which would yield a compound of Formula XVI.

Path B: a compound of Formula XIII can be reacted with sodium azide to form a compound of Formula XIIIa. This reaction can be carried out in an organic solvent, for example, tetrahydrofuran, dimethylformamide, diethylether, dioxane, or a mixture thereof.

A compound of Formula XIIIa can be reacted with compound of Formula XIIIb (wherein R₈ is alkyl) to form a compound of Formula XIIIc. This reaction can be carried out in an organic solvent and a base. Examples of organic solvents include, for example, tetrahydrofuran, dimethylformamide, diethyl ether, dioxane, or a mixture thereof. Examples of bases include sodium hydride or potassium tert-butoxide.

A compound of Formula XIIIc can be reduced to form a compound of Formula XVI. This reaction can be carried in an organic solvent, for example, methanol, ethanol, propanol, isopropylalcohol, tetrahydrofuran or ethyl acetate using catalysts for example palladium on carbon or platinum on carbon in the presence of hydrogen.

The compound of Formula XVI (wherein R₉ is hydrogen or alkyl) can be reacted with a compound of Formula IX via Path a to form a compound of Formula XVII. The reaction of a compound of Formula XVI with a compound of Formula IX (when Y is C(═O) and L is OH) to give a compound of Formula XVII (Path a) through the intermediacy of an activated derivative of a carboxylic acid, can be carried out in an organic solvent, in the presence of condensing agents and in the presence of a base. Examples of organic solvents include dichloromethane, dioxane or tetrahydrofuran. Examples of condensing agents include 1-(3-dimethylaminopropyl)-3-ethyl carbodiimide hydrochloride or dicyclohexylcarbodiimide. Examples of bases include N-methylmorpholine, diisopropylethylamine or triethylamine.

Alternatively, this reaction can be carried out through mixed anhydride by reacting compound of Formula IX with a chloroformate, for example, ethyl chloroformate or isobutylchloroformate.

The reaction of a compound of Formula XVI with a compound of Formula IX (when Y is C═O or SO₂ and L is Cl) to give a compound of Formula XVII (Path a) can be carried out in an organic solvent and in the presence of a base. Examples of organic solvents include dichloromethane, dichloroethane, chloroform, carbon tetrachloride, tetrahydrofuran or dimethylformamide. Examples of bases include pyridine, triethylamine or diisopropylethylamine.

The compound of Formula XVI can be reacted with a compound of Formula III or with a compound of Formula XI via Path b to give a compound of Formula XVIII. This reaction can be carried out in an organic solvent and optionally in the presence of a base. Examples of organic solvents include dichloromethane, dichloroethane, dimethylsulfoxide, tetrahydrofuran or dimethylformamide. Examples of bases include triethylamine, diisopropylethylamine or pyridine.

Compounds prepared using Scheme III include, for example:
1-O-Dodecyl-2,3-O-isopropylidene-6-deoxy-6-{[(4-fluorophenyl)-amino]-carbonyl}-amino-α-L-sorbofuranoside (Compound No. 39);
1-O-Dodecyl-2,3-O-isopropylidene-6-deoxy-6-[(4-fluorophenyl)-sulfonyl]-amino-α-L-sorbofuranoside (Compound No. 40);
1-O-Dodecyl-2,3-O-isopropylidene-6-deoxy-6-(ethylsulfonyl)-amino-α-L-sorbofuranoside (Compound No. 41);
1-O-Dodecyl-2,3-O-isopropylidene-6-deoxy-6-{(4-fluoro-phenyl)-carbonyl}-amino-α-L-sorbofuranoside (Compound No. 42);
1-O-Dodecyl-2,3-O-isopropylidene-6-deoxy-6-{[3-(1,3-benzodioxol-5-yl)-propanoyl]-amino}-α-L-sorbofuranoside (Compound No. 43);
1-O-Dodecyl-2,3-O-isopropylidene-6-deoxy-6-{[{(4-[2-hydroxy-2-oxo-ethyl]-phenyl)-amino}-carbonyl]-amino}-α-L-sorbofuranoside (Compound No. 44);
1-O-Dodecyl-2,3-O-isopropylidene-6-deoxy-6-{[(butyl-amino)-carbonyl]-amino}-α-L-sorbofuranoside (Compound No. 45);
1-O-Dodecyl-2,3-O-isopropylidene-6-deoxy-6-({[(4-fluoro-phenyl)-amino]-thiocarbonyl}-amino)-α-L-sorbofuranoside (Compound No. 46).
1-O-Dodecyl-2,3-O-isopropylidene-6-deoxy-6-[({[2-(carboxymethyl)phenyl]amino}-carbonyl)amino]-α-L-sorbofuranoside (Compound No. 66);
1-O-Dodecyl-2,3-O-isopropylidene-6-deoxy-6-[({[3-(carboxymethyl)phenyl]amino}-carbonyl)amino]-α-L-sorbofuranoside (Compound No. 67);
1-O-Dodecyl-2,3-O-isopropylidene-6-deoxy-6-{[(4-trifluoromethyl)benzoyl]amino}-α-L-sorbofuranoside (Compound No. 68);
1-O-Dodecyl-2,3-O-isopropylidene-6-deoxy-6-{[(4-fluorophenyl)acetyl}amino]-α-L-sorbofuranoside (Compound No. 69);
1-O-Dodecyl-2,3-O-isopropylidene-6-deoxy-6-{(3-fluorobenzoyl)amino}-α-L-sorbofuranoside (Compound No. 70);
1-O-Dodecyl-2,3-O-isopropylidene-6-deoxy-6-{(quinolin-2-ylcarbonyl)amino}-α-L-sorbofuranoside (Compound No. 71);
1-O-Dodecyl-2,3-O-isopropylidene-6-deoxy-6-{(2-thienylacetyl)amino}-α-L-sorbofuranoside (Compound No. 72);
1-O-Dodecyl-2,3-O-isopropylidene-6-deoxy-6-{[(4-methoxyphenyl)acetyl]amino}-α-L-sorbofuranoside (Compound No. 73);
1-O-Dodecyl-2,3-O-isopropylidene-6-deoxy-6-{[(3-fluorophenyl)acetyl]amino}-α-L-sorbofuranoside (Compound No. 74);
1-O-Dodecyl-2,3-O-isopropylidene-6-deoxy-6-{(3,4-dimethoxybenzoyl)amino}-α-L-sorbofuranoside (Compound No. 75);
1-O-Dodecyl-2,3-O-isopropylidene-6-deoxy-6-{(isoquinolin-1-ylcarbonyl)amino}-α-L-sorbofuranoside (Compound No. 76);
1-O-Dodecyl-2,3-O-isopropylidene-6-deoxy-6-{[4-(acetylamino)benzoyl]amino}-α-L-sorbofuranoside (Compound No. 77);
1-O-Dodecyl-2,3-O-isopropylidene-6-deoxy-6-{[(pyridin-4-yl)-carbonyl]-amino}-α-L-sorbofuranoside (Compound No. 78);
1-O-Dodecyl-2,3-O-isopropylidene-6-deoxy-6-{[(2,6-dichloropyridin-4-yl)-carbonyl]-amino}-α-L-sorbofuranoside (Compound No. 79);
1-O-Dodecyl-2,3-O-isopropylidene-6-deoxy-6-{[(quinolin-3yl)-carbonyl]-amino}-α-L-sorbofuranoside (Compound No. 80);
1-O-Dodecyl-2,3-O-isopropylidene-6-deoxy-6-{[(5-methyl-3-phenylisoxazol-4-yl)-carbonyl]-amino}-α-L-sorbofuranoside (Compound No. 81);
1-O-Dodecyl-2,3-O-isopropylidene-6-deoxy-6-{(phenyl)acetyl}-amino-α-L-sorbofuranoside (Compound No. 82);
1-O-Dodecyl-2,3-O-isopropylidene-6-deoxy-6-{[(4-chlorophenyl)acetyl]amino-α-L-sorbofuranoside (Compound No. 83);
1-O-Dodecyl-2,3-O-isopropylidene-6-deoxy-6-{[(3,5-difluorophenyl)acetyl]amino}-α-L-sorbofuranoside (Compound No. 84);
1-O-Dodecyl-2,3-O-isopropylidene-6-deoxy-6-{[(3-methoxyphenyl)acetyl]amino}-α-L-sorbofuranoside (Compound No. 85);
1-O-Dodecyl-2,3-O-isopropylidene-6-deoxy-6-{[(3-chlorophenyl)acetyl]amino}-α-L-sorbofuranoside (Compound No. 86);
1-O-Dodecyl-2,3-O-isopropylidene-6-deoxy-6-{[(2-methoxyphenyl)acetyl]amino}-α-L-sorbofuranoside (Compound No. 87);
1-O-Dodecyl-2,3-O-isopropylidene-6-deoxy-6-{[(2,4-difluorophenyl)acetyl]amino}-α-L-sorbofuranoside (Compound No. 88);
1-O-Dodecyl-2,3-O-isopropylidene-6-deoxy-6-{[(2,6-chlorophenyl)acetyl]amino}-α-L-sorbofuranoside (Compound No. 89);
1-O-Dodecyl-2,3-O-isopropylidene-6-deoxy-6-{[(4-methylphenyl)acetyl]amino}-α-L-sorbofuranoside (Compound No. 90);
1-O-Dodecyl-2,3-O-isopropylidene-6-deoxy-6-{[(3,4-difluorophenyl)acetyl]amino}-α-L-sorbofuranoside (Compound No. 91);
1-O-Dodecyl-2,3-O-isopropylidene-6-deoxy-6-{[(2,5-difluorophenyl)acetyl]amino}-α-L-sorbofuranoside (Compound No. 92);
1-O-Dodecyl-2,3-O-isopropylidene-6-deoxy-6-{[(2,4,5-trifluorophenyl)acetyl]amino}-α-L-sorbofuranoside (Compound No. 93);
1-O-Dodecyl-2,3-O-isopropylidene-6-deoxy-6-{[(3,4-dichlorophenyl)acetyl]amino}-α-L-sorbofuranoside (Compound No. 94);
1-O-Dodecyl-2,3-O-isopropylidene-6-deoxy-6-{[(4-hydroxyphenyl)acetyl]amino}-α-L-sorbofuranoside (Compound No. 95);
1-O-Dodecyl-2,3-O-isopropylidene-6-deoxy-6-{[(2-methylphenyl)acetyl]amino}-α-L-sorbofuranoside (Compound No. 96);
1-O-Dodecyl-2,3-O-isopropylidene-6-deoxy-6-{[(2-chlorophenyl)acetyl]amino}-α-L-sorbofuranoside (Compound No. 97);
1-O-Dodecyl-2,3-O-isopropylidene-6-deoxy-6-[(1,3-benzodioxol-5-ylacetyl)amino]-L-sorbofuranoside (Compound No. 98);
1-O-Dodecyl-2,3-O-isopropylidene-6-deoxy-6-{[(3-hydroxyphenyl)acetyl]amino}-α-L-sorbofuranoside (Compound No. 99);
1-O-Dodecyl-2,3-O-isopropylidene-6-deoxy-6-{[(4-hydroxy-3-fluorophenyl)acetyl]amino}-α-L-sorbofuranoside (Compound No. 100);
1-O-Dodecyl-2,3-O-isopropylidene-6-deoxy-6-{[(4-isopropylphenyl)acetyl]amino}-α-L-sorbofuranoside (Compound No. 101);
1-O-Dodecyl-2,3-O-isopropylidene-6-deoxy-6-{[biphenyl-4-ylacetyl]amino}-α-L-sorbofuranoside (Compound No. 102);
1-O-Dodecyl-2,3-O-isopropylidene-6-deoxy-6-{[(3-methylphenyl)acetyl]amino}-α-L-sorbofuranoside (Compound No. 103);
1-O-Dodecyl-2,3-O-isopropylidene-6-deoxy-6-{[(2-fluoro-6-chlorophenyl)acetyl]amino}-α-L-sorbofuranoside (Compound No. 104);
1-O-Dodecyl-2,3-O-isopropylidene-6-deoxy-6-{[(2-chloro-4-fluorophenyl)acetyl]amino}-α-L-sorbofuranoside (Compound No. 105);
1-O-Dodecyl-2,3-O-isopropylidene-6-deoxy-6-{[(4-trifluoromethoxyphenyl)acetyl]amino}-α-L-sorbofuranoside (Compound No. 106);
1-O-Dodecyl-2,3-O-isopropylidene-6-deoxy-6-{[(2-trifluoromethoxyphenyl)acetyl]amino}-α-L-sorbofuranoside (Compound No. 107);
1-O-Dodecyl-2,3-O-isopropylidene-4-O-methyl-6-deoxy-6-{[(4-fluorophenyl)acetyl]amino}-α-L-sorbofuranoside (Compound No. 108);
1-O-(2-butoxyethyl)-2,3-O-isopropylidene-6-deoxy-6-{[(3,4-difluorophenyl)acetyl]amino}-α-L-sorbofuranose (Compound No. 109);
1-O-(2-butoxyethyl)-2,3-O-isopropylidene-6-deoxy-6-{[(3,4 dichlorophenyl)acetyl]amino}-α-L-sorbofuranose (Compound No. 110);
1-O-(2-butoxyethyl)-2,3-O-isopropylidene-6-deoxy-6-{[(4-methoxyphenyl)acetyl]amino}-α-L-sorbofuranose (Compound No. 111);
1-O-(2-butoxyethyl)-2,3-O-isopropylidene-6-deoxy-6-{[(3-methoxyphenyl)acetyl]amino}-α-L-sorbofuranose (Compound No. 112); or
1-O-(2-butoxyethyl)-2,3-O-isopropylidene-6-deoxy-6-{[(4-fluorophenyl)acetyl]amino}-α-L-sorbofuranoside (Compound No. 113).

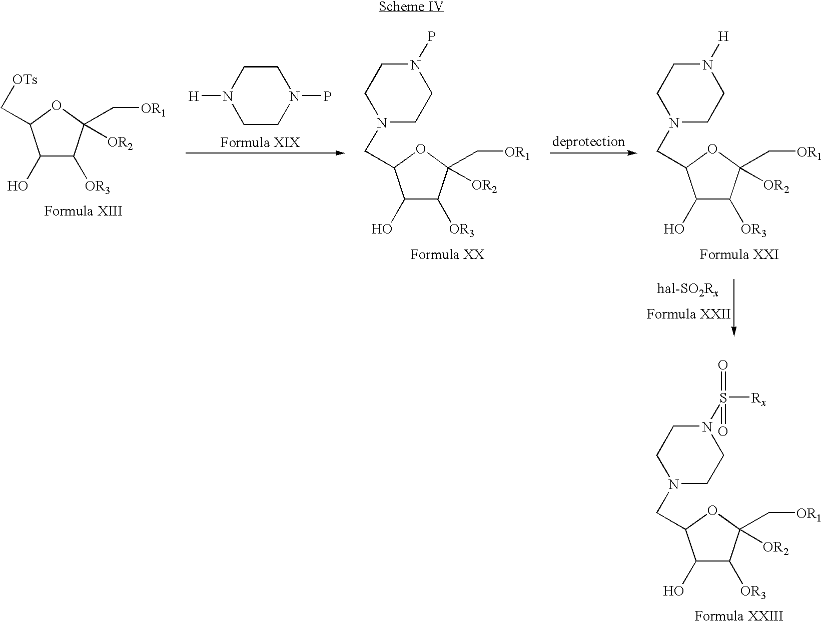

A compound of Formula XXIII can be prepared by Scheme IV. Thus, a compound of Formula XIII can be reacted with a compound of Formula XIX (wherein P is a protecting group, for example, aralkyl or acyl) to form a compound of Formula XX (wherein R₁, R₂ and R₃ is same as defined earlier).

The compound of Formula XX can be deprotected to form a compound of Formula XXI. The deprotection can be carried out in an organic solvent and under conditions of deprotection. Examples of organic solvents include methanol, ethanol, propanol, isopropylalcohol, tetrahydrofuran or ethyl acetate. Examples of conditions of deprotection include hydrogenatically utilizing palladium on carbon or under catalytic transfer hydrogenation conditions of ammonium formate and palladium on carbon.

The compound of Formula XXI can be reacted with a compound of Formula XXII (wherein hal is halogen, and Rₓ is same as defined earlier) to yield a compound of Formula XXIII. This reaction can be carried out in an organic solvent and in the presence of a base. Examples of organic solvents include dichloromethane, dichloroethane, chloroform, carbon tetrachloride, tetrahydrofuran or dimethylformamide. Examples of bases include pyridine, triethylamine or diisopropylethylamine.

Compounds prepared using Scheme IV include, for example:
Hydrochloride salt of 1-O-Dodecyl-2,3-O-isopropylidene-6-deoxy-6-{1-[4-({4-ethoxy-3-[5-{1-methyl-3-propyl-7-oxo-1,6-dihydro-pyrazolo[4,3-d]-pyrimidinyl}]-phenyl-}sulfonyl)-piperazinyl]}-α-L-sorbofuranoside (Compound No. 47).

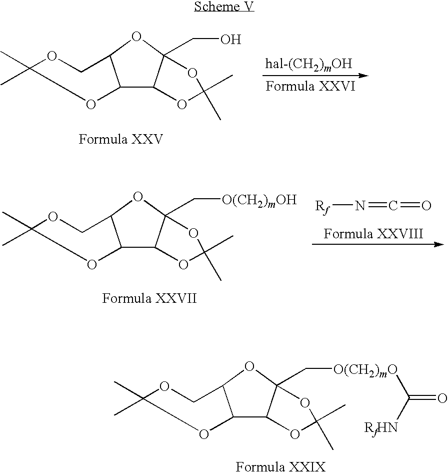

A compound of Formula XXIX can be prepared following Scheme V. Thus, a compound of Formula XXV can be reacted with a compound of Formula XXVI to form a compound of Formula XXVII. The reaction can be carried out in an organic solvent and a base in the presence of a phase transfer catalyst. Examples of organic solvents include, for example, dimethylsulfoxide or N,N-dimethylformamide. Examples of bases include, for example, potassium hydroxide or sodium hydroxide. Examples of phase transfer catalysts include, for example, tetrabutylammonium iodide or tetrabutylammonium bromide.

The compound of Formula XXVII can be reacted with a compound of Formula XXVIII (wherein R_{f} is same as described earlier) to yield a compound of Formula XXIX. The reaction can be carried out in an organic solvent, for example, dichloromethane, dichloroethane, chloroform or carbon tetrachloride.

Compounds prepared using Scheme V include, but are not limited to:
1-O-[6-{(4-Nitro-phenyl)-amino-carbonyloxy}-hexyl]-2,3;4,6-di-O-isopropylidene-α-L-sorbofuranoside (Compound No. 48);
1-O-[6-{(4-Chloro-phenyl)-amino-carbonyloxy}-hexyl]-2,3;4,6-di-O-isopropylidene-α-L-sorbofuranoside (Compound No. 49);
1-O-[6-{(4-Methoxy-phenyl-amino-carbonyloxy)-hexyl]-2,3;4,6-di-O-isopropylidene-α-L-sorbofuranoside (Compound No. 50); or
1-O-{6-[(4-Methyl-phenyl)-amino-carbonyloxy}-hexyl]-2,3;4,6-di-O-isopropylidene-α-L-sorbofuranoside (Compound No. 51).

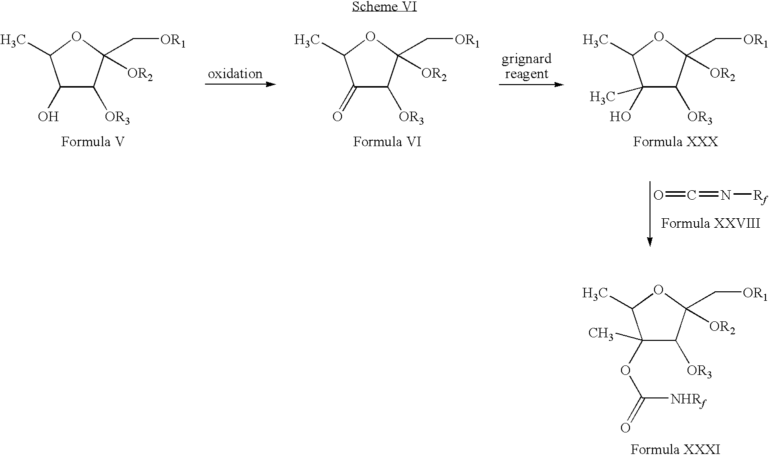

A compound of Formula XXXI can be prepared by following Scheme VI. Thus, the compound of Formula V (wherein R₁, R₂ and R₃ are same as defined earlier) can be oxidized to the compound of Formula VI.

The compound of Formula VI can be reacted with a Grignard reagent to form a compound of Formula XXX. This reaction can be carried out in an organic solvent, for example, dry tetrahydrofuran or diethylether. Examples of Grignard reagents include, for example, alkyl magnesium chloride, for example, methyl magnesium chloride.

The compound of Formula XXX can be reacted with a compound of Formula XXVIII (wherein R_{f} is same as described earlier) to form a compound of Formula XXXI. This reaction can be carried out in an organic solvent and in the presence of a base. Examples of organic solvents include, for example, dichloromethane, dichloroethane, dimethylsulfoxide, tetrahydrofuran or dimethylformamide. Examples of bases include, for example, triethylamine, diisopropylethylamine or pyridine.

Compounds prepared using Scheme V include, for example:
(4ξ)-1-O-Dodecyl-2,3-O-isopropylidene-4-C-methyl-6-deoxy-α-L-erythro-hex-2-ulofuranoside (Compound No. 52),
(4ξ)-1-O-Dodecyl-2,3-O-isopropylidene-4-C-methyl-4-O-{[(phenylsulfonyl)-amino]-carbonyl}-6-deoxy-α-L-erythro-hex-2-ulofuranoside (Compound No. 53),
(4ξ)-1-O-Dodecyl-2,3-O-isopropylidene-4-C-methyl-4-O-[{[(4-methyl-phenyl)-sulfonyl]-amino}-carbonyl]-6-deoxy-α-L-erythro-hex-2-ulofuranoside (Compound No. 54),
(4ξ)-1-O-Dodecyl-2,3-O-isopropylidene-4-C-methyl-4-O-[{[(4-chloro-phenyl)-sulfonyl]-amino}-carbonyl]-6-deoxy-α-L-erythro-hex-2-ulofuranoside (Compound No. 55),
(4ξ)-1-O-Dodecyl-2,3-O-isopropylidene-4-C-methyl-4-O-{[{2,5-dichloro-phenyl)-sulfonyl}-amino]-carbonyl}-6-deoxy-α-L-erythro-hex-2-ulofuranoside (Compound No. 56),
(4ξ)-1-O-Dodecyl-2,3-O-isopropylidene-4-C-methyl-4-O-[{[(2-methyl-phenyl)-sulfonyl]-amino}-carbonyl]-6-deoxy-α-L-erythro-hex-2-ulofuranoside (Compound No. 57),
(4ξ)-1-O-Dodecyl-2,3-O-isopropylidene-4-C-heptyl-4-O-[2-(1-piperidinyl)-ethyl]-6-deoxy-α-L-erythro-hex-2-ulofuranoside (Compound No. 58),
(4ξ)-1-O-Dodecyl-2,3-O-isopropylidene-4-C-heptyl-4-O-[2-(1-azepanyl)-ethyl]-6-deoxy-α-L-erythro-hex-2-ulofuranoside (Compound No. 59),
(4ξ)-1-O-Dodecyl-2,3-O-isopropylidene-4-C-heptyl-4-O-[2-(1-morpholinyl)-ethyl]-6-deoxy-α-L-erythro-hex-2-ulofuranoside (Compound No. 60),
(4ξ)-1-O-Dodecyl-2,3-O-isopropylidene-4-C-heptyl-4-O-[2-(1-pyrrolidinyl)-ethyl]-6-deoxy-α-L-erythro-hex-2-ulofuranoside (Compound No. 61),
(4ξ)-1-O-Dodecyl-2,3-O-isopropylidene-4-C-heptyl-4-O-heptyl-6-deoxy-α-L-erythro-hex-2-ulofuranoside (Compound No. 62),
(4ξ)-1-O-Dodecyl-2,3-O-isopropylidene-4-C-heptyl-4-O-[2-(1-dimethylamino)-propyl]-6-deoxy-α-L-erythro-hex-2-ulofuranoside (Compound No. 63),
(4ξ)-1-O-Dodecyl-2,3-O-isopropylidene-4-C-methyl-4-O-[{[(2-azepanyl)-sulfonyl]-amino}-carbonyl]-6-deoxy-α-L-erythro-hex-2-ulofuranoside (Compound No. 64)
(4ξ)-1-O-Heptyl-2,3-O-isopropylidene-4-C-methyl-4-O-[{[(4-methylphenyl)sulphonyl]amino}-carbonyl]-6-deoxy-α-L-erythro-hex-2-ulofuranoside (Compound No. 114),
(4ξ)-1-O-Heptyl-2,3-O-isopropylidene-4-C-methyl-4-O-[{[(2-methylphenyl)sulphonyl]amino}-carbonyl]-6-deoxy-α-L-erythro-hex-2-ulofuranoside (Compound No. 115),
(4ξ)-1-O-Heptyl-2,3-O-isopropylidene-4-C-methyl-4-O-[{[(4-chlorophenyl)sulphonyl]amino}-carbonyl]-6-deoxy-α-L-erythro-hex-2-ulofuranoside (Compound No. 116),
(4ξ)-1-O-Heptyl-2,3-O-isopropylidene-4-C-methyl-4-O-[{[phenylsulphonyl]amino}carbonyl]-6-deoxy-α-L erythro-hex-2-ulofuranoside (Compound No. 117), or
(4ξ)-1-O-Heptyl-2,3-O-isopropylidene-4-C-methyl-4-O-[{[(4-fluorophenyl)sulphonyl]amino}-carbonyl]-6-deoxy-α-L-erythro-hex-2-ulofuranoside (Compound No. 118).

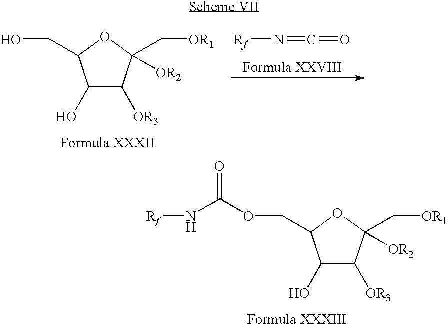

A compound of Formula XXXIII can be prepared following Scheme VII. Thus, a compound of Formula XXXII (wherein R₁, R₂ and R₃ are as described earlier) can be reacted with the compound of Formula XXVIII (wherein R_{f} is same as described earlier) to form the compound of Formula XXXIII. This reaction can be carried out in an organic solvent and optionally in the presence of a base. Examples of organic solvents include, for example, dichloromethane, dichloroethane, tetrahydrofuran or dioxane. Examples of bases include, for example, triethylamine, diisopropylethylamine or pyridine.

A particular illustrative compound prepared through Scheme VII is, for example:
1-O-Heptyl-2,3-O-isopropylidene-6-O-{[(4-methyl-phenyl)-amino]-carbonyl}-α-L-sorbofuranoside (Compound No. 65).

Wherever esters are specified in the compounds disclosed above, one of ordinary skill in the art optionally could hydrolyze them to their respective acids. For example, hydrolysis of alkyl esters (for example, ethyl, methyl or benzyl ester) to their corresponding acids can be carried out in the presence of a base (for example, lithium hydroxide, sodium hydroxide or potassium hydroxide). Alternatively, hydrolysis of benzyl esters can be carried out hydrogenatically using catalysts (for example, palladium on carbon or platinum on carbon). Esters, for example, tert-butyl, can be hydrolyzed to their corresponding acids in the presence of acid (for example, trifluoroacetic acid or hydrochloric acid).

Where specific bases, acids, solvents, condensing agents, hydrolyzing agents and other reagents are mentioned in the above schemes, it is understood that other acids, bases, solvents, condensing agents, hydrolyzing agents and other reagents known to those skilled in the art also may be used. Similarly, reaction temperatures and duration of reactions may be adjusted according to the desired needs.

Suitable salts of the compounds represented by Formula I are pharmacologically acceptable salts and can be prepared so as to solubilize the compound in aqueous medium for biological evaluations, as well as to be compatible with various dosage formulations and to aid in the bioavailability of the compounds. Examples of such salts include inorganic acid salts (e.g., hydrochloride, hydrobromide, sulfate, nitrate or phosphate), organic acid salts (e.g., acetate, tartrate, citrate, fumarate, maleate, toluenesulfonate or methanesulfonate). When free carboxylic acid groups are included in the Formula I as substituents, they may form organic and inorganic base salts (for example, tris(hydroxymethyl)aminomethane, sodium, potassium, calcium, magnesium, or ammonium and the like). These salts may be prepared by prior art techniques known to one of ordinary skill in the art, for example, treating the compound with an equivalent amount of inorganic or organic base in water.

While the present invention has been described in terms of its specific embodiments, certain modifications and equivalents will be apparent to those skilled in the art and are included within the scope of the present invention.

TABLE I

| | |
|---|---|
| | Formula I |
| 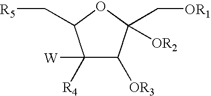 | |

wherein W is H and R₂ & R₃ together form isopropylidene radical

| Compound | | | |
|---|---|---|---|
| No. | R₁ | R₄ | R₅ |
| 1 | —C₁₀H₂₁ | 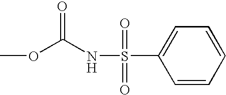 | H |
| 2 | —C₁₂H₂₅ | 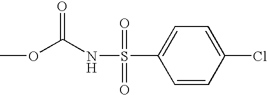 | 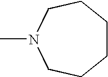 |
| 3 | —C₁₂H₂₅ | 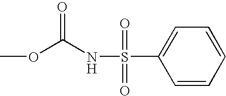 | 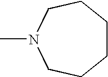 |
| 4 | —C₁₂H₂₅ | 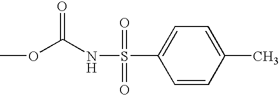 | 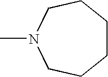 |
| 5 | —C₁₂H₂₅ | 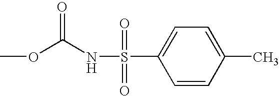 | 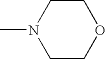 |
| 6 | —C₁₂H₂₅ | 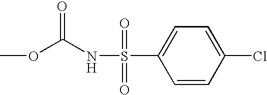 | 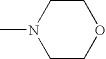 |
| 7 | —C₁₂H₂₅ | 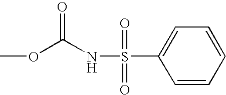 | 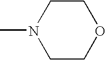 |
| 8 | —C₁₂H₂₅ | 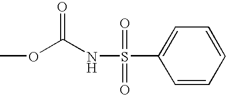 | 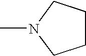 |
| 9 | —C₁₂H₂₅ | 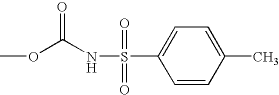 | H |
| 10* | —C₁₂H₂₅ | 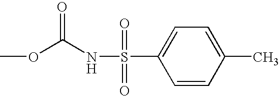 | 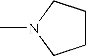 |
| 11* | —C₁₂H₂₅ | 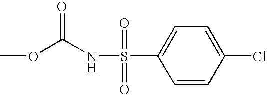 | 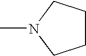 |
| 12 | —C₇H₁₅ | 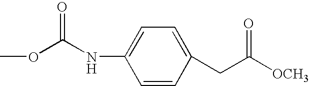 | 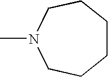 |
| 13 | —C₇H₁₅ | 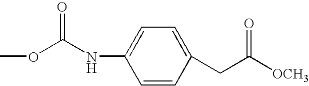 | 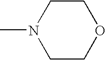 |
| 14* | —C₇H₁₅ | 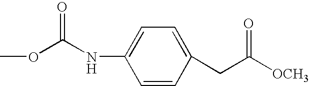 | 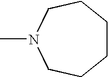 |
| 15 | —C₁₂H₂₅ | 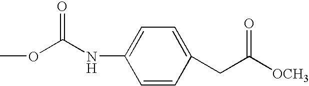 | 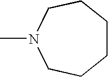 |
| 16 | —C₁₂H₂₅ | 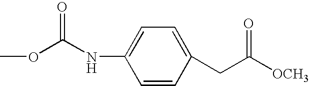 | 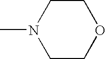 |
| 17 | —C₁₂H₂₅ | 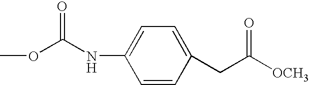 | 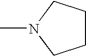 |
| 18 | —C₁₂H₂₅ | 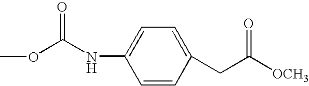 | 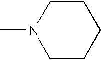 |
| 19 | —C₇H₁₅ | 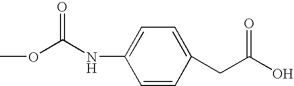 | 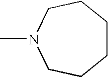 |
| 20 | —C₁₀H₂₁ | 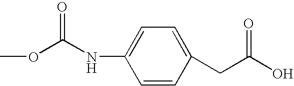 | 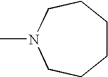 |
| 21** | —C₇H₁₅ | 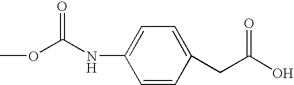 | 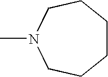 |
| 22** | —C₁₀H₂₁ | 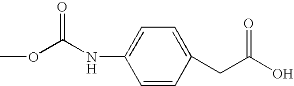 | 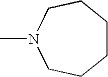 |
| 23 | —C₁₂H₂₅ | 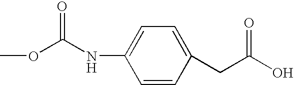 | 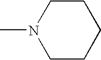 |
| 24 | —C₁₂H₂₅ | 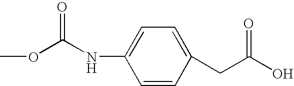 | 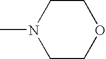 |
| 25 | —C₁₂H₂₅ | 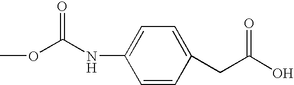 | 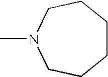 |
| 26 | —C₁₂H₂₅ | 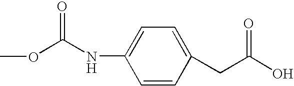 | 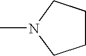 |
| 27 | —C₁₂H₂₅ | 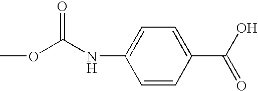 | 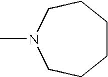 |
| 28** | —C₁₀H₂₁ | 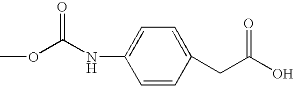 | 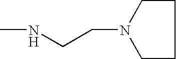 |
| 29** | —C₁₀H₂₁ | 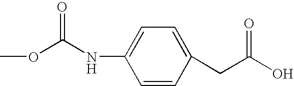 | 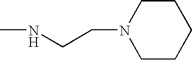 |
| 30** | —C₁₀H₂₁ | 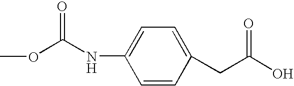 | 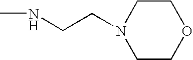 |
| 31** | —C₁₀H₂₁ | 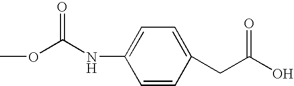 | 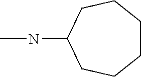 |
| 32 | —C₁₂H₂₅ | 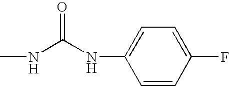 | H |
| 33 | —C₁₂H₂₅ | 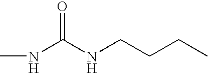 | H |
| 34 | —C₁₂H₂₅ | 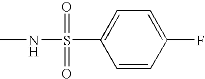 | H |
| 35 | —C₁₂H₂₅ | 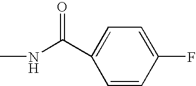 | H |
| 36 | —C₁₂H₂₅ | 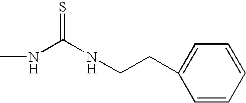 | H |
| 37 | —C₁₂H₂₅ | 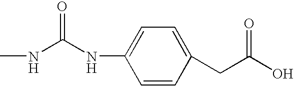 | H |
| 38 | —C₁₂H₂₅ | 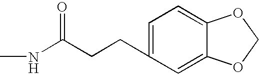 | H |
| 39 | —C₁₂H₂₅ | —OH | 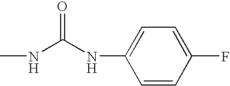 |
| 40 | —C₁₂H₂₅ | —OH | 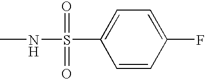 |
| 41 | —C₁₂H₂₅ | —OH | 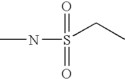 |
| 42 | —C₁₂H₂₅ | —OH | 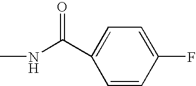 |
| 43 | —C₁₂H₂₅ | —OH | 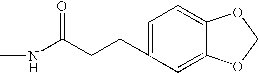 |
| 44 | —C₁₂H₂₅ | —OH | 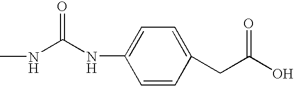 |
| 45 | —C₁₂H₂₅ | —OH | 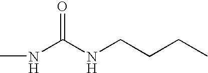 |
| 46 | —C₁₂H₂₅ | —OH | 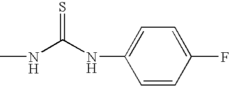 |
| 47 | —C₁₂H₂₅ | —OH | 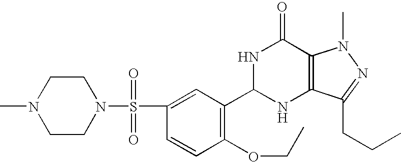 |
| 65. | —C₇H₁₅ | —OH | 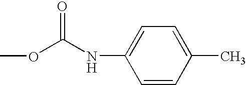 |
| 66 | —C₁₂H₂₅ | —OH | 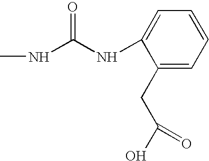 |
| 67 | —C₁₂H₂₅ | —OH | 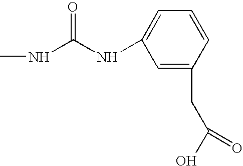 |
| 68 | —C₁₂H₂₅ | —OH | 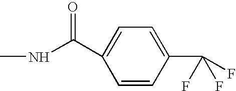 |
| 69 | —C₁₂H₂₅ | —OH | 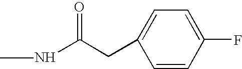 |
| 70 | —C₁₂H₂₅ | —OH | 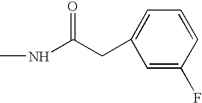 |
| 71 | —C₁₂H₂₅ | —OH | 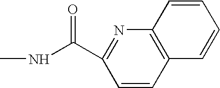 |
| 72 | —C₁₂H₂₅ | —OH | 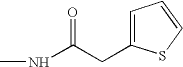 |
| 73 | —C₁₂H₂₅ | —OH | 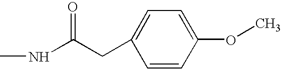 |
| 74 | —C₁₂H₂₅ | —OH | 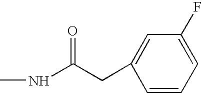 |
| 75 | —C₁₂H₂₅ | —OH | 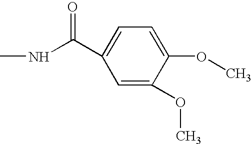 |
| 76 | —C₁₂H₂₅ | —OH | 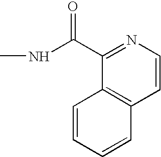 |
| 77 | —C₁₂H₂₅ | —OH | 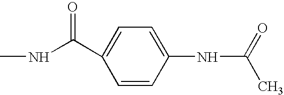 |
| 78 | —C₁₂H₂₅ | —OH | 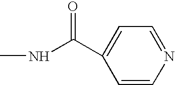 |
| 79 | —C₁₂H₂₅ | —OH | 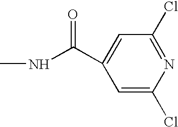 |
| 8013420 | —C₁₂H₂₅ | —OH | 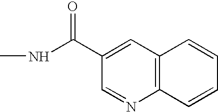 |
| 81 | —C₁₂H₂₅ | —OH | 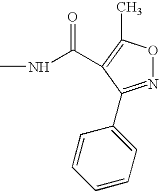 |
| 82 | —C₁₂H₂₅ | —OH | 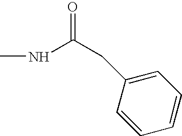 |
| 83 | —C₁₂H₂₅ | —OH | 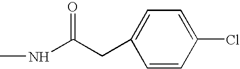 |
| 84 | —C₁₂H₂₅ | —OH | 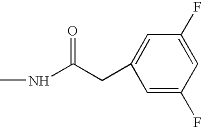 |
| 85 | —C₁₂H₂₅ | —OH | 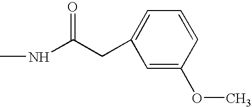 |
| 86 | —C₁₂H₂₅ | —OH | 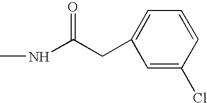 |
| 87 | —C₁₂H₂₅ | —OH | 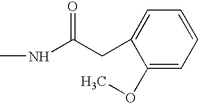 |
| 88 | —C₁₂H₂₅ | —OH | 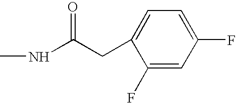 |
| 89 | —C₁₂H₂₅ | —OH | 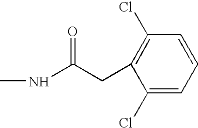 |
| 90 | —C₁₂H₂₅ | —OH | 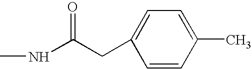 |
| 91 | —C₁₂H₂₅ | —OH | 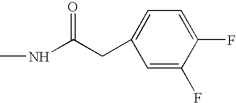 |
| 92 | —C₁₂H₂₅ | —OH | 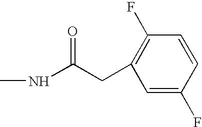 |
| 93 | —C₁₂H₂₅ | —OH | 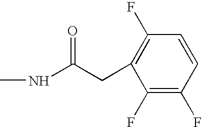 |
| 94 | —C₁₂H₂₅ | —OH | 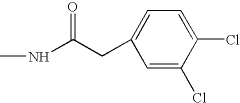 |
| 95 | —C₁₂H₂₅ | —OH | 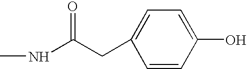 |
| 96 | —C₁₂H₂₅ | —OH | 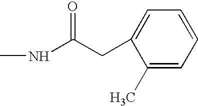 |
| 97 | —C₁₂H₂₅ | —OH | 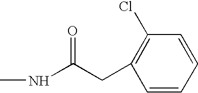 |
| 98 | —C₁₂H₂₅ | —OH | 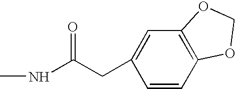 |
| 99 | —C₁₂H₂₅ | —OH | 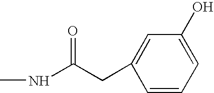 |
| 100 | —C₁₂H₂₅ | —OH | 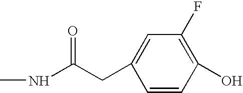 |
| 101 | —C₁₂H₂₅ | —OH | 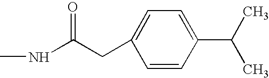 |
| 102 | —C₁₂H₂₅ | —OH | 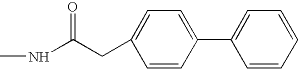 |
| 103 | —C₁₂H₂₅ | —OH | 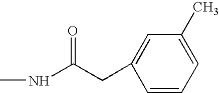 |
| 104 | —C₁₂H₂₅ | —OH | 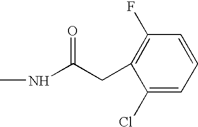 |
| 105 | —C₁₂H₂₅ | —OH | 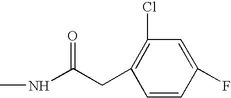 |
| 106 | —C₁₂H₂₅ | —OH | 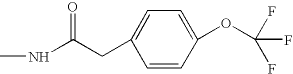 |
| 107 | —C₁₂H₂₅ | —OH | 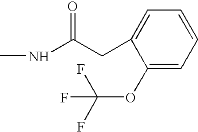 |
| 108 | —C₁₂H₂₅ | —OMe | 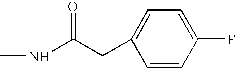 |
| 109 | —C₂H₄OC₄H₉ | —OH | 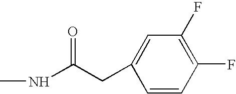 |
| 110 | —C₂H₄OC₄H₉ | —OH | 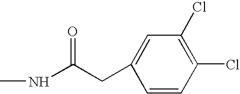 |
| 111 | —C₂H₄OC₄H₉ | —OH | 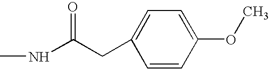 |
| 112 | —C₂H₄OC₄H₉ | —OH | 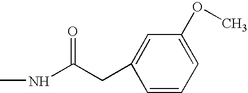 |
| 113 | —C₂H₄OC₄H₉ | —OH | 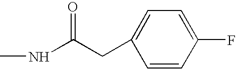 |

*hydrochloride salt
**tris(hydroxymethyl) aminomethane salt

TABLE II

| | |
|---|---|
| | Formula I |
| 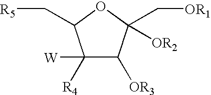 | |

wherein W is H and R₂ & R₃ and R_{c} (when R₄ is OR_{c} ) and
R_{z} (when R₅ is OR_{z} ) together form isopropylidene radical

| Compound No. | R₁ |
|---|---|
| 48 | 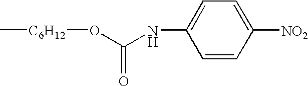 |
| 49 | 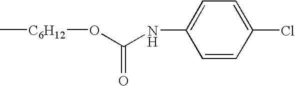 |

TABLE III

| | |
|---|---|
| | Formula I |
| 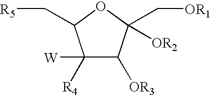 | |

wherein R₂ & R₃ together are forming
isopropylidene radical and R₅ is hydrogen

| Compound No. | R₁ | R₄ | W |
|---|---|---|---|
| 52. | C₁₂H₂₅ | —OH | CH₃ |
| 53. | C₁₂H₂₅ | 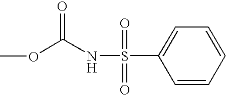 | CH₃ |
| 54. | C₁₂H₂₅ | 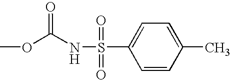 | CH₃ |
| 55. | C₁₂H₂₅ | 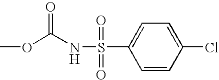 | CH₃ |
| 56. | C₁₂H₂₅ | 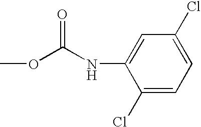 | CH₃ |
| 57. | C₁₂H₂₅ | 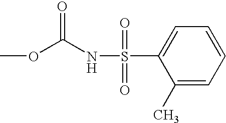 | CH₃ |
| 58. | C₁₂H₂₅ | 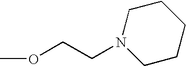 | C₇H₁₅ |
| 59. | C₁₂H₂₅ | 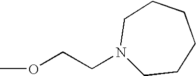 | C₇H₁₅ |
| 60. | C₁₂H₂₅ | 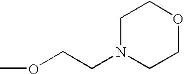 | C₇H₁₅ |
| 61. | C₁₂H₂₅ | 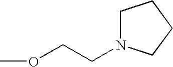 | C₇H₁₅ |
| 62. | C₁₂H₂₅ | —O—C₇H₁₅ | C₇H₁₅ |
| 63. | C₁₂H₂₅ | 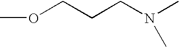 | C₇H₁₅ |
| 64. | C₁₂H₂₅ | 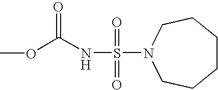 | C₇H₁₅ |
| 114. | C₇H₁₅ | 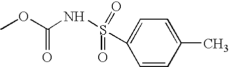 | CH₃ |
| 115. | C₇H₁₅ | 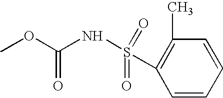 | CH₃ |
| 116. | C₇H₁₅ | 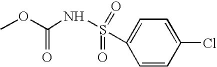 | CH₃ |
| 117. | C₇H₁₅ | 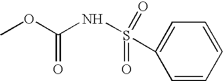 | CH₃ |
| 118 | C₇H₁₅ | 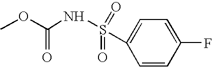 | CH₃ |

The examples mentioned below demonstrate the general synthetic procedure as well as the specific preparation for the preparation for the preferred compound. The examples are given to illustrate particular aspects of the invention and do not limit the scope of the present invention.

### EXAMPLES

### Example 1

### Synthesis of 4-methoxy-3-(1-methyl-7-oxo-3-propyl-6,7-dihydro-1H-pyrazalo[4,3-d]pyrimidin-5-yl)-benzenesulfonyl chloride

The compound 1-methyl-5-(2-methoxy-phenyl)-3-propyl-1,6-dihydro-pyrazolo[4,3-d]pyrimidin-7-one was added in small portions to a cooled solution of chlorosulfonic acid. The temperature of the reaction mixture was maintained at about 10-15° C. and stirred for 5-6 hours. Thionyl chloride was slowly added and the reaction mixture was stirred for an additional 20 hours. Reaction mixture was poured onto crushed ice followed by the addition of dichloromethane and stirred for 15 minutes. The reaction mixture was filtered through celite pad. The organic layer was collected and the solvent was evaporated off. The solvent was dried over anhydrous sodium sulfate. The residue thus obtained was triturated with hexane and filtered to obtain the title compound.

### Example 1A

### Synthesis of 1-O-(2-butoxyethyl)-2,3-O-isopropylidene-6-tosyl-α-L-sorbofuranoside

### Step a: Synthesis of Methanesulphonic Acid 2-butoxy-ethyl Ester

Triethylamine (3.5 ml) and methanesulphonylchloride (1.4 ml) were added to a solution of 2-butoxyethanol (2 gm) in dichloromethane (40 ml) at 0° C. and stirred for 1 hour at the same temperature. The reaction mixture was taken in water and extracted with dichloromethane, the combined organic layers were dried over anhydrous sodium sulfate. The dichloromethane was evaporated under the reduced pressure to get the desired crude title compound (3.8 gm).

### Step b: Synthesis of 1-O-(2-butoxyethyl)-2,3;4,6-di-O-isopropylidene-α-L-sorbofuranoside

Sodium hydride (406 mg) was added to a solution of 2,3;4,6-di-O-isopropylidene-α-L-sorbofuranoside (4 gm) in dimethylformamide (50 ml) at 0° C. and stirred for about 10 mins. To the reaction mixture was added methanesulphonic acid 2-butoxy-ethyl ester (3 gm) obtained from step a above at the same temperature and further stirred for 2 hrs at room temperature. The reaction mixture was taken in water and extracted with ethyl acetate, the combined organic layers were dried over anhydrous sodium sulfate. The solvent was evaporated under reduced pressure to yield crude product, which was purified by column chromatography, using 20% ethylacetate-hexane as eluant to yield the title compound (3 gm).

### Step c: Synthesis of 1-O-(2-butoxyethyl)-2,3-O-isopropylidene-α-L-sorbofuranoside

HClO₄ (1.7 gm) was added to a solution of 1-O-(2-butoxyethyl)-2,3;4,6-di-O-isopropylidene-α-L-sorbofuranoside (3 gm) obtained from step b above in tetrahydrofuran (20 ml) at 0° C., and stirred for 4 hrs at the same temperature. Excess HClO₄ was neutralized by addition of dilute sodium hydroxide solution. The reaction mixture was extracted with ethyl acetate and the combined organic layers were dried over anhydrous sodium sulfate. The solvent was evaporated under reduced pressure to yield crude product, which was purified by column chromatography, using 30% ethyl acetate-hexane as eluant to yield the title compound (2.6 gm).

### Step d: Synthesis of 1-O-(2-butoxyethyl)-2,3-O-isopropylidene-6-tosyl-α-L-sorbofuranoside

Triethylamine (1.7 ml) and p-Toluenesulfonylchloride (1.7 gm) were added to a solution of 1-O-(2-butoxyethyl)-2,3-O-isopropylidene-α-L-sorbofuranoside (2.6 gm) obtained from step c above in dichloromethane (60 ml) at room temperature and stirred for 12 hrs.

Then reaction mixture was taken in water and extracted with dichloromethane. The combined organic layers were dried over anhydrous sodium sulfate. The solvent was evaporated under reduced pressure to yield crude product, which was purified by column chromatography, using 30% ethyl acetate-hexane as eluant to yield the title compound (2.0 gm).

NMR.(400 MHz, CDCl₃): δ 7.82 (d, 2H, 8 Hz), 7.33 (d, 2H, 8 Hz), 4.41 (s, 1H), 4.35 (s, 1H), 4.32-4.33 (m, 1H), 4.21-4.22 (m, 2H), 3.65-3.75 (m, 5H), 3.37-3.39 (m, 4H), 2.44 (s, 3H), 1.50-1.54 (m, 2H), 1.46 (s, 3H), 1.35 (s, 3H), 0.88-0.99 (m, 3H)

LCMS: (m/z) 497 (M+Na)

### Scheme I:

### Example 2

### Synthesis of 1-O-Decyl-2,3-O-isopropylidene-4-O-{[(phenyl-sulfonyl)-amino]-carbonyl}-6-deoxy-6-(4-morpholinyl)-α-L-sorbofuranoside (Compound No. 1)

Benzenesulfonyl isocyanate (0.057 mL) was added to a solution of 1-O-Decyl-2,3-O-isopropylidene-6-deoxy-6-(4-morpholinyl)-α-L sorbofuranoside (150 mg) in dichloromethane (10 mL) at 0° C., stirred for 1 hour at this temperature and followed by stirring at room temperature for 3 hours. The solvent was evaporated under reduced pressure and the residue was purified over silica gel column using 30% ethyl acetate-hexane as eluent to yield the title compound (210 mg).

¹H NMR (300 MHz, CDCl₃): δ 8.04 (2H, d, J=9 Hz), 7.61 (3H, m), 4.98 (1H, s), 4.45 (2H, s), 3.62-3.46 (5H, m), 1.59 (2H, m), 1.48 (3H, s), 1.36 (3H, s), 1.27 (14H, m), 1.06 (3H, d, J=6 Hz), 0.87 (3H, d, J=6 Hz)

LCMS (m/e): 550 (M⁺+Na)

Analogs of 1-O-Decyl-2,3-O-isopropylidene-4-O-{[(phenyl-sulfonyl)-amino]-carbonyl}-6-deoxy-6-(4-morpholinyl)-α-L-sorbofuranoside (Compound No. 1) listed below can be prepared by replacing benzene sulfonyl isocyanate with the appropriate isocyanate, as applicable in each case:
1-O-Dodecyl-2,3-O-isopropylidene-4-O-{[(4-chloro-phenyl)-sulfonylamino]-carbonyl}-6-deoxy-6-(1-azepanyl)-α-L-sorbofuranoside (Compound No. 2),
1-O-Dodecyl-2,3-O-isopropylidene-4-O-{[phenyl-sulfonylamino]-carbonyl}-6-deoxy-6-(1-azepanyl)-α-L-sorbofuranoside (Compound No. 3),
1-O-Dodecyl-2,3-O-isopropylidene-4-O-{[(4-methyl-phenyl)-sulfonylamino]-carbonyl}-6-deoxy-6-(1-azepanyl)-α-L-sorbofuranoside (Compound No. 4),
1-O-Dodecyl-2,3-O-isopropylidene-4-O-{[(4-methyl-phenyl)-sulfonylamino]-carbonyl}-6-deoxy-6-(4-morpholinyl)-O-L-sorbofuranoside (Compound No. 5),
1-O-Dodecyl-2,3-O-isopropylidene-4-O-{[(4-chloro-phenyl)-sulfonylamino]-carbonyl}-6-deoxy-6-(4-morpholinyl)-α-L-sorbofuranoside (Compound No. 6),
1-O-Dodecyl-2,3-O-isopropylidene-4-O-{[(phenyl-sulfonyl)-amino]-carbonyl}-6-deoxy-6-(4-morpholinyl)-α-L-sorbofuranoside (Compound No. 7),
1-O-Dodecyl-2,3-O-isopropylidene-4-O-{[(phenylsulfonyl)-amino]-carbonyl}-6-deoxy-6-(1-pyrrolidinyl)-α-L-sorbofuranoside (Compound No. 8),
1-O-Dodecyl-2,3-O-isopropylidene-4-O-{[(4-methyl-phenyl)-sulfonylamino]-carbonyl}-6-deoxy-α-L-sorbofuranoside (Compound No. 9),
Hydrochloride salt of 1-O-Dodecyl-2,3-O-isopropylidene-4-O-{[(4-methyl-phenyl)-sulfonylamino]-carbonyl}-6-deoxy-6-(1-pyrrolidinyl)-α-L-sorbofuranoside. (Compound No. 10),
1-O-Dodecyl-2,3-O-isopropylidene-4-O-{[(4-chloro-phenyl)-sulfonylamino]-carbonyl}-6-deoxy-6-(1-pyrrolidinyl)-α-L-sorbofuranoside. (Compound No. 11).

### Example 3

### Synthesis of 1-O-Heptyl-2,3-O-isopropylidene-4-O-{[4-(2-methoxy-2-oxo-ethyl)-phenyl]-amino}-carbonyl-6-deoxy-6-(1-azepanyl)-α-L-sorbofuranoside (Compound No. 12)

Methyl 4-isocyanatophenyl acetate (0.57 g) was added dropwise with continuous stirring to a solution of 1-O-heptyl-2,3-O-isopropylidene-6-deoxy-6-(1-azepanyl)-α-L-sorbofuranoside (1.0 g) (prepared as described in U.S. Pat. No. 5,637,570) in dichloromethane (20 mL) at 0-5° C. The reaction mixture was allowed to warm to room temperature and after 24 hours, dichloromethane was removed under reduced pressure to obtain crude product. The crude residue thus obtained was purified by column chromatography using 30% ethyl acetate-hexane as eluent to yield the title compound (1.40 g).

¹HNMR (CDCl₃, 300 MHz): δ 7.32-7.35 (2H, m), 7.26-7.12 (2H, m), 6.78 (1H, bs, NH), 5.23 (1H, bs), 4.55-4.50 (2H, m), 3.67-3.62 (4H, m), 3.57-3.47 (5H, m), 2.80 (2H, d, 5.9 Hz), 2.72-2.67 (4H, m), 1.77 (1H, bs), 1.591.48 (10H, m), 1.38 (3H, s), 1.24 (10H, bs), 0.84 (3H, t, 6.3 Hz).

Analogs of 1-O-Heptyl-2,3-O-isopropylidene-4-O-{[4-(2-ethoxy-2-oxo-ethyl)-phenyl]-amino}-carbonyl-6-deoxy-6-(1-azepanyl)-α-L-sorbofuranoside (Compound No. 12) described below can be prepared by replacing methyl 4-isocyanatophenylacetate with the appropriate isocyanate, as applicable in each case.
1-O-Heptyl-2,3-O-isopropylidene-4-O-{[4-(2-methoxy-2-oxo-ethyl)-phenyl]-amino}-carbonyl-6-deoxy-6-(4-morpholinyl)-α-L-sorbofuranoside (Compound No. 13),
Hydrochloride salt of 1-O-heptyl-2,3-O-isopropylidene-4-O-{[4-(2-methoxy-2-oxo-ethyl)-phenyl]-amino}-carbonyl-6-deoxy-6-(1-azepanyl)-α-L-sorbofuranoside (Compound No. 14),
1-O-Dodecyl-2,3-O-isopropylidene-4-O-{[4-(2-methoxy-2-oxo-ethyl)-phenyl]-amino}-carbonyl-6-deoxy-6-(1-azepanyl)-α-L-sorbofuranoside (Compound No. 15),
1-O-Dodecyl-2,3-O-isopropylidene-4-O-{[4-(2-methoxy-2-oxo-ethyl)-phenyl]-amino}-carbonyl-6-deoxy-6-(4-morpholinyl)-α-L-sorbofuranoside (Compound No. 16),
1-O-Dodecyl-2,3-O-isopropylidene-4-O-{[4-(2-methoxy-2-oxo-ethyl)-phenyl]-amino}-carbonyl-6-deoxy-6-(1-pyrrolidinyl)-α-L-sorbofuranoside (Compound No. 17),
1-O-Dodecyl-2,3-O-isopropylidene-4-O-{[4-(2-methoxy-2-oxo-ethyl)-phenyl]-amino}-carbonyl-6-deoxy-6-(1-piperidinyl)-α-L-sorbofuranoside (Compound No. 18).

### Example 4

### Synthesis of 1-O-Heptyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 19)

1N sodium hydroxide (50 mL) was added to a solution of 1-O-Heptyl-2,3-O-isopropylidene-4-O-{[4-(2-ethoxy-2-oxo-ethyl)-phenyl]-amino}-carbonyl-6-deoxy-6-(1-azepanyl)-α-L-sorbofuranoside (1.20 g) (obtained from Example 2) in methanol (20 mL), and the reaction mixture was stirred at 50° C. After 5 hours, methanol was removed under reduced pressure and the reaction mixture was treated with dilute HCl until ˜pH 5 was obtained. The solid thus separated was extracted with ethyl acetate. The organic extracts were washed with water and brine and dried over anhydrous sodium sulfate. The residue thus obtained was purified by column chromatography using 30% ethyl acetate-hexane as eluent to furnish the title compound (0.95 g).

¹HNMR (CDCl₃, 300 MHz): δ 7.26-7.00 (4H, m), 6.5 (1H, bs), 5.15 (1H, bs), 4.60 (1H, bs), 3.69-3.63 (1H, m), 3.58 (3H, m), 3.26-3.21 (1H, m), 3.10-3.04 (3H, bs), 2.89 (3H, m), 2.78 (3H, m), 1.76 (4H, bs), 1.62-1.52 (4H, m), 1.48-1.38 (95H, m), 1.38-1.35 (3H, m), 1.31-1.25 (8H, bs), 0.86 (3H, t, 7.7 Hz).

Analogs of 1-O-Heptyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 19) described below can be prepared by hydrolyzing the respective esters to their corresponding acids.
1-O-Decyl-2,3-O-isopropylidene-4-O-{[(4-[2-hydroxy-2-oxo-ethyl]-phenyl)-amino]-carbonyl}-6-deoxy-6-(1-azepanyl)-α-L-sorbofuranoside (Compound No. 20),
Tris salt of 1-O-Heptyl-2,3-O-isopropylidene-4-O-{[4-(2-hydroxy-2-ethyl)-phenyl]-amino}-carbonyl-6-deoxy-6-(1-azepanyl)-α-L-sorbofuranoside (Compound No. 21),
Tris salt of-1-O-decyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 22),
1-O-Dodecyl-2,3-O-isopropylidene-4-O-{[4-(2-hydroxy-2-oxo-ethyl)-phenyl]-amino}-carbonyl-6-deoxy-6-(1-piperidinyl)-α-L-sorbofuranoside (Compound No. 23),
1-O-Dodecyl-2,3-O-isopropylidene-4-O-{[4-(2-hydroxy-2-oxo-ethyl)-phenyl]-amino}-carbonyl-6-deoxy-6-(4-morpholinyl)-α-L-sorbofuranoside (Compound No. 24),
1-O-Dodecyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 25),
1-O-Dodecyl-2,3-O-isopropylidene-4-O-{[4-(2-hydroxy-2-oxo-ethyl)-phenyl]-amino}-carbonyl-6-deoxy-6-(1-pyrrolidinyl)-α-L-sorbofuranoside (Compound No. 26),
1-O-Dodecyl-2,3-O-isopropylidene-4-O-[{4-(2-hydroxy-2-oxy-ethyl)-phenyl}-amino]-carbonyl-6-deoxy-6-(1-azepanyl)-α-L-sorbofuranoside (Compound No. 27).

### Example 5

### Synthesis of Tris salt of-1-O-Decyl-2,3-O-isopropylidene-4-O-[(4-{2-hydroxy-2-oxo-ethyl}-phenyl)-amino]-carbonyl-6-deoxy-6-[2-(1-pyrrolidinyl)-ethyl]-amino-α-L-sorbofuranoside (Compound No. 28)

### Step a: Synthesis of 1-O-Decyl-2,3-O-isopropylidene-4-O-[(4-{2-methoxy-2-oxo-ethyl}phenyl)-amino]-carbonyl-6-deoxy-6-[2-(1-pyrrolidinyl)-ethyl]-amino-α-L-sorbofuranoside

Methyl 4-isocyanatophenyl acetate (0.50 g) was added to a solution of 1-O-Decyl-2,3-O-isopropylidene-6-deoxy-6-[2-(1-pyrrolidinyl)-ethyl]-amino-α-L-sorbofuranoside (1.0 g) (prepared as described in U.S. Pat. No. 5,637,570) in dichloromethane (20 mL) at 0-5° C., with continuous stirring. The reaction mixture was allowed to warm to room temperature. After 6 hours, dichloromethane (30 mL) was added to the reaction mixture and organic layer was washed with water and brine, and then dried over anhydrous sodium sulfate. The solvent was removed under reduced pressure and the residue thus obtained was purified by column chromatography using 50% ethyl acetate-methanol as eluent to yield the title compound (1.16 g).

### Step b: Synthesis of 1-O-Decyl-2,3-O-isopropylidene-4-O-[(4-{2-hydroxy-2-oxo-ethyl}-phenyl)-amino]-carbonyl-6-deoxy-6-[2-(1-pyrrolidinyl)-ethyl]amino-α-L-sorbofuranoside

0.5 N aqueous sodium hydroxide solution (30 mL) was added to a solution of 1-O-Decyl-2,3-O-isopropylidene-4-O-[(4-{2-methoxy-2-oxo-ethyl}-phenyl)-amino]-carbonyl-6-deoxy-6-[2-(1-pyrrolidinyl)-ethyl]-amino-α-L-sorbofuranoside (0.6 g) (obtained from step a above) in methanol (5 mL), and the reaction mixture was stirred at 50° C. After 3 hours, the solvent was evaporated and residue was dissolved in water. The aqueous solution was acidified to ˜pH 5 with concentrated HCl and extracted with ethylacetate. The organic extracts were washed with water and brine, and dried over anhydrous sodium sulfate. The solvent was removed at reduced pressure to yield the title compound (0.34 g).

### Step c: Synthesis of Tris salt of-1-O-Decyl-2,3-O-isopropylidene-4-O-[(4-{2-hydroxy-2-oxo-ethyl}-phenyl)-amino]-carbonyl-6-deoxy-6-[2-(1-pyrrolidinyl)-ethyl]amino-α-L-sorbofuranoside

Tris (hydroxymethyl)aminomethane (0.057 g) was added to a solution of 1-O-Decyl-2,3-O-isopropylidene-4-O-[(4-{2-hydroxy-2-oxo-ethyl}-phenyl)-amino]-carbonyl-6-deoxy-6-[2-(1-pyrrolidinyl)-ethyl]amino-α-L-sorbofaranoside (0.3 g) (obtained from step b above) in ethanol (20 mL) at room temperature and the reaction mixture was stirred for one hour. Ethanol was removed at reduced pressure to yield the title compound as a hygroscopic solid (34 mg).

¹H NMR (DMSO, 300 MHz): δ 7.34 (2H, d, 8.2 Hz), 7.13 (2H, d, 8.2 Hz), 4.35 (1H, s), 4.31 (1H, bs), 4.06 (1H, bs), 3.73-3.12 (16H, m), 1.87 (4H, bs), 1.50-1.03 (22H, m), 0.85 (3H, bs)

Analogs of Tris salt of-1-O-Decyl-2,3-O-isopropylidene-4-O-[(4-{2-hydroxy-2-oxo-ethyl}-phenyl)-amino]-carbonyl-6-deoxy-6-[2-(1-pyrrolidinyl)-ethyl]amino-α-L-sorbofuranoside (Compound No. 28) described below can be prepared by replacing 2-(1-pyrrolidinyl)-ethylamine in step a with the appropriate amine, as applicable in each case.
Tris salt of 1-O-Decyl-2,3-O-isopropylidene-4-O-[{4-(2-hydroxy-2-oxo-ethyl)-phenyl}-amino]-carbonyl-6-deoxy-6-[2-(1-piperidinyl)-ethyl]amino-α-L-sorbofuranoside (Compound No. 29),
Tris salt of 1-O-Decyl-2,3-O-isopropylidene-4-O-{[4-(2-hydroxy-2-oxo-ethyl)-phenyl]-amino}-carbonyl-6-deoxy-6-[2-(4-morpholinyl)-ethyl]-amino-α-L-sorbofuranoside (Compound No. 30),
Tris salt of 1-O-Decyl-2,3-O-isopropylidene-4-O-{[4-(2-hydroxy-2-oxo-ethyl)-phenyl]-amino}-carbonyl-6-deoxy-6-[2-(1-cycloheptyl-amino)-ethyl]-amino-α-L-sorbofuranoside (Compound No. 31).

### Scheme II

### Example 6

### Synthesis of (4ξ)-1-O-Dodecyl-2,3-O-isopropylidene-4,6-di-deoxy-4-[{[(4-fluoro-phenyl)-amino]-carbonyl}-amino]-α-L-erythro-hex-2-ulofuranoside (Compound No. 32)

### Step a: Synthesis of (4ξ)-1-O-Dodecyl-2,3-O-isopropylidene-4-oxo-6-deoxy-α-L-erythro-hex-2-ulofuranoside

Pyridinium dichromate (1.52 g) and acetic anhydride (1.15 mL) was added to a solution of 1-O-Dodecyl-2,3-O-isopropylidene-6-deoxy-α-L-sorbofuranoside (1.5 g) (prepared as described in U.S. Pat. No. 5,637,570) in dichloromethane (10 mL) at room temperature. The reaction mixture then was refluxed for 2-3 hours. The solvent was evaporated under reduced pressure and the resulting residue was dissolved in ethyl acetate and filtered over silica gel. The filtrate was evaporated under reduced pressure to yield the title compound as yellow oil (1.3 g).

¹H NMR (300 MHz, CDCl₃): δ 4.59 (1H, q, 6 Hz), 4.32 (1H, s), 3.53-3.48 (4H, m), 1.59-1.52 (5H, m), 1.43 (3H, s), 1.38-1.26 (21H, m), 0.90-0.86 (3H, m).

LCMS (m/e): 571 (M⁺+Na)

### Step b: Synthesis of (4ξ)-1-O-Dodecyl-2,3-O-isopropylidene-4,6-dideoxy-4-hydroxy-imino-α-L-sorbofuranoside

Hydroxylamine hydrochloride (0.74 g) was added to a solution of (4ξ)-1-O-Dodecyl-2,3-O-isopropylidene-4-oxo-6-deoxy-α-L-erythro-hex-2-ulofuranoside (1.3 g) (obtained from step a above) in pyridine-ethanol (1:1, 3 mL) at room temperature. The reaction mixture was refluxed at about 75° C. for 3 hours, after which the solvent was evaporated under reduced pressure and the residue was dissolved in water and extracted with ethyl acetate. The combined organic extracts were washed with water and brine and dried over anhydrous sodium sulfate. The solvent was evaporated under reduced pressure to yield crude oil, and purified by column chromatography, using 10% ethyl acetate-hexane as eluent to yield the title compound (1 g).

¹H NMR (300 MHz, CDCl₃): δ 7.41 (1H, d, 16 Hz), 5.29-5.14 (1H, m), 5.01-4.90 (1H, m), 3.58-3.49 (4H, m), 1.56-1.38 (13H, m), 1.26 (16H, m), 0.88-0.86 (3H, m).

LCMS (m/e): 424 (M⁺+K)

### Step c: Synthesis of (4ξ)-1-O-Dodecyl-2,3-O-isopropylidene-4,6-dideoxy-4-amino-α-L-erythro-hex-2-ulofuranoside

Lithium aluminum hydride (0.29 g) was added portion-wise to a solution of (4ξ)-1-O-Dodecyl-2,3-O-isopropylidene-4,6-dideoxy-4-hydroxy-imino-α-L-sorbofuranoside (1 g) (obtained from step b above) in dry tetrahydrofuran with vigorous stirring at room temperature and further stirred overnight at room temperature. The reaction mixture then was quenched with a few drops of 10% aqueous sodium hydroxide solution, diluted with ethyl acetate, and the resulting residue was filtered over celite. The filtrate was evaporated under reduced pressure to yield the title compound as yellow oil (800 mg).

### Step d: Synthesis of (4ξ)-1-O-Dodecyl-2,3-O-isopropylidene-4,6-di-deoxy-4-[{[(4-fluoro-phenyl)-amino]-carbonyl}-amino]-α-L-erythro-hex-2-ulofuranoside (Compound No. 32)

4-fluorophenyl isocyanate (0.03 mL) was added to a solution of (4ξ)-1-O-Dodecyl-2,3-O-isopropylidene-4,6-dideoxy-4-amino-α-L-erythro-hex-2-ulofuranoside (100 mg) (obtained from step c above) in dichloromethane (3 mL) at 0° C. and stirred for 2 hours. The reaction mixture then was concentrated and the resulting crude oil was purified by column chromatography using 15% ethyl acetate-hexane as eluent to yield the title compound (90 mg).

¹H NMR (300 MHz, CDCl₃): δ7.31-7.28 (2H, m), 7.00 (2H, t, 8.5 Hz), 6.39 (1H, s), 5.04 (1H, d, 9 Hz), 4.55 (1H, d, 4.5 Hz), 4.02-3.88 (2H, m), 3.60-3.47 (4H, m), 1.62-1.50 (5H, m), 1.44-1.25 (27H, m), 0.89-0.85 (3H, m).

LCMS (m/e): 531 (M⁺+Na)

### Example 7

### Synthesis of (4ξ)-1-O-Dodecyl-2,3-O-isopropylidene-4,6-dideoxy-4-{[(4-fluoro-phenyl)-carbonyl]-amino}-α-L-erythro-hex-2-ulofuranoside (Compound No. 35)

4-fluoro-benzoyl chloride was added to a solution having 100 mg of a compound obtained from step c of Example 6 (i.e., (4ξ)-1-O-Dodecyl-2,3-O-isopropylidene-4,6-dideoxy-4-amino-α-L-erythro-hex-2-ulofuranoside), in dichloromethane (3 mL) at 0° C. The reaction mixture was stirred for 2-3 hours and then taken into water and extracted with dichloromethane. The combined organic extracts were washed with aqueous sodium bicarbonate, water and brine and dried over anhydrous sodium sulfate. Solvent was evaporated under reduced pressure to obtain crude yellow oil, which was purified by column chromatography using 15% ethyl acetate-hexane as eluent to furnish the title compound (75 mg).

¹H NMR (300 MHz, CDCl₃): δ 7.83-7.79 (2H, m), 7.14 (2H, t, 9 Hz), 6.37 (1H, d, 9 Hz), 4.60 (1H, d, 3 Hz), 4.30 (1H, m), 4.06 (1H, m), 3.61-3.50 (4H, m), 1.59-1.49 (5H, m), 1.41-1.36 (6H, m), 1.26 (18H, m), 0.90-0.85 (3H, m).

LCMS (m/e): 494 (M⁺+1)

### Example 8

### Synthesis of (4ξ)-1-O-Dodecyl-2,3-O-isopropylidene-4,6-di-deoxy-4-{[(butyl-amino)-carbonyl}-amino}-α-L-erythro-hex-2-ulofuranose (Compound No. 33)

N-butyl isocyanate (0.03 mL) was added to a solution of (4ξ)-1-O-Dodecyl-2,3-O-isopropylidene-4,6-dideoxy-4-amino-α-L-erythro-hex-2-ulofuranoside (100 mg) (obtained from step c of Example 6) in dichloromethane (3 mL) at 0° C. The reaction mixture was stirred for 3 hours and solvent was evaporated under reduced pressure. The crude residue thus obtained was purified by column chromatography using 20% ethyl acetate-hexane as eluent to yield the title compound (85 mg).

¹H NMR (300 MHz, CDCl₃): δ 4.56-4.51 (2H, m), 4.25 (1H, bs), 3.91 (2H, m), 3.60-3.47 (4H, m), 3.19-3.15 (2H, m), 1.58-1.46 (8H, m), 1.38-1.30 (4H, m), 1.26 (21H, m), 0.95-0.86 (6H, m).

LCMS (m/e): 471 (M⁺+1)

### Example 9

### Synthesis of (4ξ)-1-O-Dodecyl-2,3-O-isopropylidene-4,6-dideoxy-4-{[(4-fluoro-phenyl)-sulfonyl]-amino}-α-L-erythro-hex-2-ulofuranoside (Compound No. 34)

Triethylamine (0.04 mL) and 4-fluorobenzene sulfonyl chloride (53 mg) were added to a solution of (4ξ)-1-O-Dodecyl-2,3-O-isopropylidene-4,6-dideoxy-4-amino-α-L-erythro-hex-2-ulofuranoside (100 mg) (obtained from step c of Example 6) in dichloromethane (3 mL) at 0° C. and stirred for 3 hours. The reaction mixture was taken into water and extracted with ethyl acetate. The organic layer was washed with water and brine and dried over anhydrous sodium sulfate. The solvent was evaporated under reduced pressure and the resulting crude oil was purified by column chromatography using 15% ethyl acetate-hexane as eluent to yield the title compound (80 mg).

¹H NMR (300 MHz, CDCl₃): δ 7.94-7.90 (2H, m), 7.18 (2H, t, 9 Hz), 5.07 (1H, d, 9 Hz), 4.02 (1H, d, 6 Hz), 3.89 (1H, m), 3.48-3.40 (4H, m), 3.22 (1H, m), 1.56-1.49 (5H, m), 1.27-1.22 (24H, m), 0.90-0.86 (3H, m).

LCMS (m/e): 547 (M⁺+18)

### Example 10

### Synthesis of (4ξ)-1-O-Dodecyl-2,3-O-isopropylidene-4,6-dideoxy-4-{[3-(1,3-benzodionol-5-yl)-propanoyl]-amino}-α-L-erythro-hex-2-ulofuranoside (Compound No. 38)

N-methylmorpholine (0.03 mL, 0.29 mmol) and 1-hydroxy benzotriazole (40 mg, 0.29 mmol) were added to a solution of (4ξ)-1-O-Dodecyl-2,3-O-isopropylidene-4,6-dideoxy-4-amino-α-L-erythro-hex-2-ulofuranoside (100 mg) (obtained from step c of Example 6) and 3-benzo[1,3]-dioxol-5-yl-propionic acid (52 mg, 0.26 mmol) in dimethylformamide (3 mL) at 0° C. After 0.5 hours, N-(dimethylaminopropyl)-N-ethyl carbodiimide hydrochloride (132 mg, 0.67 mmol) was added to the reaction mixture and stirred overnight at room temperature. The reaction mixture was quenched with water, extracted with ethyl acetate, and the combined organic extracts were washed with water and brine and dried over anhydrous sodium sulfate. The solvent was evaporated under reduced pressure to yield crude yellow oil which was purified by column chromatography using 20% ethyl acetate-hexane as eluent to furnish the title compound (80 mg).

¹H NMR (300 MHz, CDCl₃): δ 6.71-6.64 (3H, m), 5.92 (2H, s), 5.62 (1H, d, 9 Hz), 4.45 (1H, d, 4.5 Hz), 4.09 (1H, m), 3.90 (1H, m), 3.58-3.46 (4H, m), 2.91-2.86 (2H, m), 2.51-2.46 (2H, m), 1.57-1.50 (5H, m), 1.25-1.10 (24H, m), 0.89-0.85 (3H, m).

LCMS (m/e): 570 (M⁺+Na)

### Example 11

### Synthesis of (4ξ)-1-O-Dodecyl-2,3-O-isopropylidene-4,6-di-deoxy-4-[{[(4-[2-hydroxy-2-oxo-ethyl]-phenyl)-amino]-carbonyl}-amino]-α-L-erythro-hex-2-ulofuranoside (Compound No. 37)

### Step a: Synthesis of (4ξ)-1-O-Dodecyl-2,3-O-isopropylidene-4,6-di-deoxy-4-[{[(4-[2-methoxy-2-oxo-ethyl]-phenyl)-amino]-carbonyl}-amino]-α-L-erythro-hex-2-ulofuranoside

(4-phenoxycarbonylamino-phenyl)-acetic acid methyl ester (77 mg) was added to a solution of (4ξ)-1-O-Dodecyl-2,3-O-isopropylidene-4,6-dideoxy-4-amino-α-L-erythro-hex-2-ulofuranoside (100 mg) (obtained from step c of Example 6) in dimethyl sulfoxide (3 mL) and triethylamine (0.04 mL) at 0° C. The reaction mixture was stirred at room temperature for 2 hrs, taken into water and extracted with ethyl acetate. The combined organic extracts were washed with water and brine and dried over anhydrous sodium sulfate. Solvent was evaporated under reduced pressure and the residue thus obtained was purified by column chromatography using 15% ethyl acetate-hexane as eluent to yield the title compound (95 mg).

¹HNMR (300 MHz, CDCl₃): δ 7.28-7.19 (4H, m), 6.40 (1H, bs), 5.08 (1H, d, 9 Hz), 4.56 (1H, d, 6 Hz), 4.02-3.92 (2H, m), 3.68 (3H, s), 3.61-3.49 (6H, m), 1.59 (2H, m), 1.51 (3H, s), 1.38-1.25 (24H, m), 0.88-0.85 (3H, m).

LCMS (m/e): 585 (M⁺+Na)

### Step b: Synthesis of (4ξ)-1-O-Dodecyl-2,3-O-isopropylidene-4,6-di-deoxy-4-[{[(4-[2-hydroxy-2-oxo-ethyl]-phenyl)-amino]-carbonyl}-amino]-α-L-erythro-hex-2-ulofuranoside

Lithium hydroxide monohydrate (7 mg, 0.17 mmol) was added to a solution of (4ξ)-1-O-Dodecyl-2,3-O-isopropylidene-4,6-di-deoxy-4-[{[(4-[2-methoxy-2-oxo-ethyl]-phenyl)-amino]-carbonyl}-amino]-α-L-erythro-hex-2-ulofuranoside (95 mg, 0.17 mmol) (obtained from step a above) in tetrahydrofuran:methanol:water (3:1:1, 5 mL) at 0° C. The reaction mixture was stirred for 2 hours. The solvent was evaporated under reduced pressure and the resulting crude mass was taken into water and extracted with ethyl acetate. The aqueous layer was acidified with aqueous sodium hydrogen sulfate and then extracted with ethyl acetate. The combined organic extracts were washed with water and brine and dried over anhydrous sodium sulfate. The solvent was evaporated under reduced pressure to yield the title compound (65 mg).

¹HNMR (300 MHz, CDCl₃): δ 8.67 (1H, s), 7.30 (2H, d, 9 Hz), 7.10 (2H, d, 9 Hz), 6.17 (1H, d, 9 Hz), 4.44 (1H, s), 3.88-3.80 (2H, m), 3.53-3.45 (6H, m), 1.48-1.32 (5H, m), 1.23-1.16 (24H, m), 0.86-0.82 (3H, m).

LCMS (m/e): 571 (M⁺+Na)

### Example 12

### Synthesis of (4ξ)-1-O-Dodecyl-2,3-O-isopropylidene-4,6-di-deoxy-4-[{[(2-phenylethyl)-amino]-thiocarbonyl}-amino]-α-L-erythro-hex-2-ulofuranoside (Compound No. 36)

Triethylamine (0.04 mL) and 2-phenylethylisothiocyanate (0.04 mL) was added to a solution of (4ξ)-1-O-Dodecyl-2,3-O-isopropylidene-4,6-dideoxy-4-amino-α-L-erythro-hex-2-ulofuranoside (100 mg) (obtained from step c of Example 6) in dichloromethane (3 mL) at room temperature and then the reaction mixture was refluxed for 3 hours. The solvent was evaporated under reduced pressure and the resulting crude oil was purified by column chromatography using 20% ethyl acetate-hexane as eluent to yield the title compound (95 mg).

¹H NMR (300 MHz, CDCl₃): δ 7.36-7.21 (5H, m), 5.74 (1H, d, 9 Hz), 4.56 (2H, m), 3.94-3.89 (1H, m), 3.69-3.48 (6H, m), 2.95-2.90 (2H, m), 1.54 (5H, m), 1.38-1.26 (21H, m), 0.90-0.85 (3H, m).

LCMS (m/e): 535 (M⁺+1)

### Scheme III

### Example 13

### Synthesis of 1-O-Dodecyl-2,3-O-isopropylidene-6-deoxy-6-{[(4-fluorophenyl)-amino]-carbonyl}-amino-α-L-sorbofuranoside (Compound No. 39)

### Step a: Synthesis of 1-O-Dodecyl-2,3-O-isopropylidene-6-deoxy-6-benzylamino-α-L-sorbofuranoside

Benzylamine (3 mL) was added to a solution of 1-O-Dodecyl-2,3-O-isopropylidene-6-tosyl-α-L-sorbofuranoside (5 g) and the reaction mixture was heated for about 2 hours at 110° C. The benzylamine was distilled out under reduced pressure and the residue thus obtained was purified over a silica gel column using 25% ethyl acetate-hexane as a eluent to yield the title compound (4.17 g).

¹H NMR (300 MHz, CDCl₃): δ 7.20-7.35 (5H, m, Aromatic), 4.42 (1H, s), 4.33 (1H, q, J=6 Hz), 4.22 (1H, d, J=3 Hz), 3.79 (2H, ABq), 3.64 (2H, dd), 3.45-3.52 (2H, m), 3.22 (1H, dd), 2.99 (1H, dd), 1.51-1.55 (5H, m), 1.37 (3H, s), 1.29 (18H, bs), 0.87 (3H, t, J=3 Hz).

LCMS (m/e): 478.25 (M⁺+1)

### Step b: Synthesis of 1-O-Dodecyl-2,3-O-isopropylidene-6-deoxy-6-amino-α-L-sorbofuranoside

10% Pd/C (2 g) was added to a solution of 1-O-Dodecyl-2,3-O-isopropylidene6-deoxy-6-benzylamino-α-L-sorbofuranoside (4.1 g) (obtained from step a above) in methanol (20 mL). The reaction mixture was shaken using a Parr apparatus at 60 psi for 12 hours at room temperature. The reaction mixture was filtered over celite and the filtrate was concentrated under reduced pressure to yield the title compound (2.5 g).

¹H NMR (300 MHz, CDCl₃): δ 4.42 (1H, s), 4.20-4.24 (2H, m), 3.60-3.71 (4H, m), 3.21-3.22 (1H, dd, 15 Hz, 3 Hz), 3.08-3.10 (1H, dd, 15 Hz, 6 Hz), 1.57-1.61 (5H, m), 1.36 (3H, s), 1.25 (18H, bs), 0.87 (3H, t, 6 Hz).

LCMS (m/e): 388.24 (M⁺+1)

### Step c: Synthesis of 1-O-Dodecyl-2,3-O-isopropylidene-6-deoxy-6-{[(4-fluorophenyl)-amino]-carbonyl}-amino-α-L-sorbofuranoside (Compound No. 39)

4-fluorophenyl isocyanate (35.4 mg) was added to a solution of 1-O-Dodecyl-2,3-O-isopropylidene-6-deoxy-6-amino-α-L-sorbofuranoside (100 mg) (obtained from step b above) in dichloromethane (10 mL) at 0° C. and the reaction mixture was allowed to warm to room temperature and stirred for 2 hours. The reaction mixture was concentrated under reduced pressure and the residue was purified over silica gel (100-200 mesh) column using 40% ethyl acetate-hexane as eluent to yield the title compound (130 mg).

¹H NMR (300 MHz, CDCl₃): δ 7.2 (2H, m), 6.99 (2H, t, 9 Hz), 4.49 (1H, bs), 4.46 (1H, s), 4.11-4.33 (3H, m), 3.44-3.74 (9H, m), 1.41-1.55 (5H, m), 1.35 (3H, s), 1.25 (18H, bs), 0.85 (3H, t, 6 Hz).

LCMS (m/e): 525.32 (M⁺+1)

### Example 14

### Synthesis of 1-O-Dodecyl-2,3-O-isopropylidene-6-deoxy-6-({[(4-fluoro-phenyl)-amino]-thiocarbonyl}-amino)-α-L-sorbofuranoside (Compound No. 46)

4-fluorophenyl isothiocyanate (39.5 mg) and triethylamine (0.01 mL) was added to a solution of 1-O-Dodecyl-2,3-O-isopropylidene-6-deoxy-6-amino-α-L-sorbofuranoside (100 mg) obtained from step b of Example 13 in dichloromethane (5 mL). The reaction mixture was stirred at room temperature for one hour. The reaction mixture was concentrated under reduced pressure and the residue was purified over a silica gel column using 15% ethyl acetate-hexane as eluent to yield the title compound (130 mg).

¹H NMR (300 MHz, CDCl₃): δ 7.26-7.08 (4H, m), 4.77 (2H, m), 4.47-4.42 (1H, m), 4.06 (1H, s), 3.72-3.69 (1H, m), 3.54-3.46 (4H, m), 1.55-1.11 (26H, m), 0.88 (3H, t, 6 Hz).

LCMS (m/e): 541 (M⁺+1)

### Example 15

### Synthesis of 1-O-Dodecyl-2,3-O-isopropylidene-6-deoxy-6-[(4-fluorophenyl)-sulfonyl]-amino-α-L-sorbofuranoside (Compound No. 40)

Triethylamine (0.036 mL) and 4-fluorobenzene sulfonylchloride (50 mg) was added to a solution of 1-O-Dodecyl-2,3-O-isopropylidene-6-deoxy-6-amino-α-L-sorbofuranoside (100 mg) obtained from step b of Example 13 in dichloromethane (5 mL) at 0° C. The reaction mixture was stirred for 3 hours at room temperature. The reaction mixture then was taken into distilled water and extracted with dichloromethane. The combined organic layer was washed with water and brine and dried over anhydrous sodium sulfate. The solvent was evaporated under reduced pressure and the residue was purified over a silica gel column using 10% ethyl acetate-hexane as eluent to yield the title compound (75 mg).

¹H NMR (300 MHz, CDCl₃) δ: 7.87-7.92 (2H, m), 7.16-7.26 (2H, m), 4.37 (1H, s), 4.24-4.28 (1H, m), 3.99-4.04 (1H, dd, 8 Hz, 3z), 3.65-3.70 (2H, m), 3.48-3.53 (2H, d, 6 Hz), 3.23-3.28 (2H, t, 6 Hz), 1.45-1.58 (5H, m), 1.31 (3H, s), 1.25 (18H, bs), 0.88 (3H, t, 6 Hz).

LCMS (m/e): 546.27 (M⁺+1)

### Example 16

### Synthesis of 1-O-Dodecyl-2,3-O-isopropylidene-6-deoxy-6-{(4-fluoro-phenyl)-carbonyl}-amino-α-L-sorbofuranoside (Compound No. 42)

4-fluorobenzoyl chloride (0.03 mL) was added to a solution of 1-O-Dodecyl-2,3-O-isopropylidene-6-deoxy-6-amino-α-L-sorbofuranoside (100 mg) obtained from step b of Example 13 in dichloromethane (2 mL) at 0° C. The reaction mixture was stirred for one hour at room temperature. The reaction mixture then was concentrated under reduced pressure and the residue was purified over a silica gel (100-200 mesh) column using 15% ethyl acetate-hexane as eluent to yield the title compound (58 mg).

¹H NMR (300 MHz, CDCl₃): δ 7.77-7.81 (2H, m), 7.08-7.26 (2H, t, 9 Hz), 6.47 (1H, bs), 4.40-4.46 (2H, m), 4.03-4.09 (2H, m), 3.88-3.90 (1H, m), 3.74-3.77 (1H, d, 9 Hz), 3.53-3.62 (4H, m), 1.50-1.58 (5H, m), 1.35 (3H, s), 1.25 (18H, bs), 0.88 (3H, t, 6 Hz).

LCMS (m/e): 510.39 (M⁺+1)

Analogs of 1-O-Dodecyl-2,3-O-isopropylidene-6-deoxy-6-{(4-fluoro-phenyl)-carbonyl}-amino-α-L-sorbofuranoside (Compound No. 42) described below can be prepared by replacing 4-fluorobenzoyl chloride with the appropriate chlorides, as applicable in each case.
1-O-Dodecyl-2,3-O-isopropylidene-6-deoxy-6-{[(4-trifluoromethyl)benzoyl]amino}-α-L-sorbofuranoside (Compound No. 68),
1-O-Dodecyl-2,3-O-isopropylidene-6-deoxy-6-{[(4-fluorophenyl)acetyl}amino]-α-L-sorbofuranoside (Compound No. 69),
1-O-Dodecyl-2,3-O-isopropylidene-6-deoxy-6-{(3-fluorobenzoyl)amino}-α-L-sorbofuranoside (Compound No. 70),
1-O-Dodecyl-2,3-O-isopropylidene-6-deoxy-6-{(quinolin-2-ylcarbonyl)amino}-α-L-sorbofuranoside (Compound No. 71),
1-O-Dodecyl-2,3-O-isopropylidene-6-deoxy-6-{(2-thienylacetyl)amino}-α-L-sorbofuranoside (Compound No. 72),
1-O-Dodecyl-2,3-O-isopropylidene-6-deoxy-6-{[(4-methoxyphenyl)acetyl]amino}-α-L-sorbofuranoside (Compound No. 73),
1-O-Dodecyl-2,3-O-isopropylidene-6-deoxy-6-{[(3-fluorophenyl)acetyl]amino}-α-L-sorbofuranoside (Compound No. 74),
1-O-Dodecyl-2,3-O-isopropylidene-6-deoxy-6-{(3,4-dimethoxybenzoyl)amino}-α-L-sorbofuranoside (Compound No. 75),
1-O-Dodecyl-2,3-O-isopropylidene-6-deoxy-6-{(isoquinolin-1-ylcarbonyl)amino}-α-L-sorbofuranoside (Compound No. 76),
1-O-Dodecyl-2,3-O-isopropylidene-6-deoxy-6-{[4-(acetylamino)benzoyl]amino}-α-L-sorbofuranoside (Compound No. 77),
1-O-Dodecyl-2,3-O-isopropylidene-6-deoxy-6-{[(pyridin-4-yl)-carbonyl]-amino}-α-L-sorbofuranoside (Compound No. 78),
1-O-Dodecyl-2,3-O-isopropylidene-6-deoxy-6-{[(2,6-dichloropyridin-4-yl)-carbonyl]-amino}-α-L-sorbofuranoside (Compound No. 79),
1-O-Dodecyl-2,3-O-isopropylidene-6-deoxy-6-{[(quinolin-3yl)-carbonyl]-amino}-α-L-sorbofuranoside (Compound No. 80),
1-O-Dodecyl-2,3-O-isopropylidene-6-deoxy-6-{[(5-methyl-3-phenylisoxazol-4-yl)-carbonyl]-amino}-α-L-sorbofuranoside (Compound No. 81),
1-O-Dodecyl-2,3-O-isopropylidene-6-deoxy-6-{(phenyl)acetyl}-amino-α-L-sorbofuranoside (Compound No. 82),
1-O-Dodecyl-2,3-O-isopropylidene-6-deoxy-6-{[(4-chlorophenyl)acetyl]amino-α-L-sorbofuranoside (Compound No. 83),
1-O-Dodecyl-2,3-O-isopropylidene-6-deoxy-6-{[(3,5-difluorophenyl)acetyl]amino}-α-L-sorbofuranoside (Compound No. 84),
1-O-Dodecyl-2,3-O-isopropylidene-6-deoxy-6-{[(3-methoxyphenyl)acetyl]amino}-α-L-sorbofuranoside (Compound No. 85),
1-O-Dodecyl-2,3-O-isopropylidene-6-deoxy-6-{[(3-chlorophenyl)acetyl]amino}-α-L-sorbofuranoside (Compound No. 86), 1-O-Dodecyl-2,3-O-isopropylidene-6-deoxy-6-{[(2-methoxyphenyl)acetyl]amino}-α-L-sorbofuranoside (Compound No. 87), 1-O-Dodecyl-2,3-O-isopropylidene-6-deoxy-6-{[(2,4-difluorophenyl)acetyl]amino}-α-L-sorbofuranoside (Compound No. 88),
1-O-Dodecyl-2,3-O-isopropylidene-6-deoxy-6-{[2,6-chlorophenyl)acetyl]amino}-α-L-sorbofuranoside (Compound No. 89),
1-O-Dodecyl-2,3-O-isopropylidene-6-deoxy-6-{[(4-methylphenyl)acetyl]amino}-α-L-sorbofuranoside (Compound No. 90),
1-O-Dodecyl-2,3-O-isopropylidene-6-deoxy-6-{[(3,4-difluorophenyl)acetyl]amino}-α-L-sorbofuranoside (Compound No. 91),
1-O-Dodecyl-2,3-O-isopropylidene-6-deoxy-6-{[(2,5-difluorophenyl)acetyl]amino}-α-L-sorbofuranoside (Compound No. 92),
1-O-Dodecyl-2,3-O-isopropylidene-6-deoxy-6-{[(2,4,5-trifluorophenyl)acetyl]amino}-α-L-sorbofuranoside (Compound No. 93),
1-O-Dodecyl-2,3-O-isopropylidene-6-deoxy-6-{[(3,4-dichlorophenyl)acetyl]amino}-α-L-sorbofuranoside (Compound No. 94),
1-O-Dodecyl-2,3-O-isopropylidene-6-deoxy-6-{[(4-hydroxyphenyl)acetyl]amino}-α-L-sorbofuranoside (Compound No. 95),
1-O-Dodecyl-2,3-O-isopropylidene-6-deoxy-6-{[(2-methylphenyl)acetyl]amino}-α-L-sorbofuranoside (Compound No. 96),
1-O-Dodecyl-2,3-O-isopropylidene-6-deoxy-6-{[(2-chlorophenyl)acetyl]amino}-α-L-sorbofuranoside (Compound No. 97),
1-O-Dodecyl-2,3-O-isopropylidene-6-deoxy-6-[(1,3-benzodioxol-5-ylacetyl)amino]-L-sorbofuranoside (Compound No. 98),
1-O-Dodecyl-2,3-O-isopropylidene-6-deoxy-6-{[(3-hydroxyphenyl)acetyl]amino}-α-L-sorbofuranoside (Compound No. 99),
1-O-Dodecyl-2,3-O-isopropylidene-6-deoxy-6-{[(4-hydroxy-3-fluorophenyl)acetyl]amino}-α-L-sorbofuranoside (Compound No. 100),
1-O-Dodecyl-2,3-O-isopropylidene-6-deoxy-6-{[(4-isopropylphenyl)acetyl]amino}-α-L-sorbofuranoside (Compound No. 101),
1-O-Dodecyl-2,3-O-isopropylidene-6-deoxy-6-{[biphenyl-4-ylacetyl]-amino}-α-L-sorbofuranoside (Compound No. 102),
1-O-Dodecyl-2,3-O-isopropylidene-6-deoxy-6-{[(3-methylphenyl)acetyl]amino}-α-L-sorbofuranoside (Compound No. 103),
1-O-Dodecyl-2,3-O-isopropylidene-6-deoxy-6-{[(2-fluoro-6-chlorophenyl)acetyl]amino}-α-L-sorbofuranoside (Compound No. 104),
1-O-Dodecyl-2,3-O-isopropylidene-6-deoxy-6-{[(2-chloro-4-fluorophenyl)acetyl]amino}-α-L-sorbofuranoside (Compound No. 105),
1-O-Dodecyl-2,3-O-isopropylidene-6-deoxy-6-{[(4-trifluoromethoxyphenyl)acetyl]amino}-α-L-sorbofuranoside (Compound No. 106), or
1-O-Dodecyl-2,3-O-isopropylidene-6-deoxy-6-{[(2-trifluoromethoxyphenyl)acetyl]amino}-α-L-sorbofuranoside (Compound No. 107).

### Example 17

### Synthesis of 1-O-Dodecyl-2,3-O-isopropylidene-6-deoxy-6-{[3-(1,3-benzodionol-5-yl)-propanoyl]-amino}-α-L-sorbofuranoside (Compound No. 43)

3-(3,4-methylenedioxyphenyl)-propionic acid (50 mg), followed by N-methylmorpholine (62 mg) and 1-hydroxybenzotriazole (38 mg) were added to a solution of 1-O-Dodecyl-2,3-O-isopropylidene-6-deoxy-6-amino-α-L-sorbofuranoside (100 mg) obtained from step b of Example 13 in N,N-dimethylformamide (3 mL) at 0° C. The reaction mixture was stirred for 30 min at 0° C. N-(dimethylaminopropyl)-N-ethyl carbodiimide hydrochloride (56 mg) was added to the reaction mixture and the reaction mixture was stirred for 24 hours at room temperature. The reaction mixture was taken in distilled water and extracted with ethyl acetate. The combined organic layer was washed with distilled water and brine and dried over anhydrous sodium sulfate. The solvent was evaporated under reduced pressure and the residue was purified over silica gel column using 30% ethyl acetate-hexane as eluent to yield the title compound (87 mg).

¹H NMR (300 MHz, CDCl₃): δ 6.62-6.74 (3H, m), 5.92 (2H, s), 4.43 (2H, s), 4.20 (1H, m), 3.95-4.06 (2H, m), 3.51-3.71 (5H, m), 2.88 (2H, 2H, 6 Hz), 2.43 (2H, t, 6 Hz), 1.58 (2H, m), 1.50 (3H, s), 1.35 (3H, s), 1.25 (18H, bs), 0.88 (3H, t, 6 Hz).

LCMS (m/e): 564.37 (M⁺+1)

### Example 18

### Synthesis of 1-O-Dodecyl-2,3-O-isopropylidene-6-deoxy-6-{[(butyl-amino)-carbonyl]-amino}-α-L-sorbofuranoside (Compound No. 45)

N-butyl isocyanate (0.03 mL) was added to a solution of 1-O-Dodecyl-2,3-O-isopropylidene-6-deoxy-6-amino-α-L-sorbofuranoside (100 mg) obtained from step b of Example 13 in dichloromethane (5 mL) at 0° C. The reaction mixture was warmed to room temperature stirred for one hour. The reaction mixture was concentrated under reduced pressure and the residue was purified over silica gel column using 15% ethyl acetate-hexane as eluent to yield the title compound (80 mg).

¹HNMR (300 MHz, CDCl₃): δ 4.45 (1H, s), 4.25 (1H, m), 4.06-4.05 (1H, d, 3 Hz), 3.71-3.51 (5H, m), 3.25 (1H, m), 3.14 (2H, t, 6 Hz), 1.57-1.55 (2H, m), 1.51-1.24 (28H, m), 0.94-0.84 (6H, m).

LCMS (m/e): 509.4 (M⁺+23)

### Example 19

### Synthesis of 1-O-Dodecyl-2,3-O-isopropylidene-6-deoxy-6-{[{(4-[2-hydroxy-2-oxo-ethyl]-phenyl)-amino}-carbonyl]-amino}-α-L-sorbofuranoside (Compound No. 44)

### Step a: Synthesis of 1-O-Dodecyl-2,3-O-isopropylidene-6-deoxy-6-{[{(4-[2-ethoxy-2-oxo-ethyl]-phenyl)-amino}-carbonyl]-amino}-α-L-sorbofuranoside

Triethylamine (0.071 mL) and (4-phenoxycarbonylamino-phenyl)-acetic acid methyl ester (147 mg) was added to a solution of 1-O-Dodecyl-2,3-O-isopropylidene-6-deoxy-6-amino-α-L-sorbofuranoside (200 mg) obtained from step b of Example 13 in dry tetrahydrofuran (5 mL) at room temperature. The reaction mixture was stirred for 3 hours at room temperature and then heated to and maintained at 50° C. overnight. The reaction mixture was taken into distilled water and extracted with ethyl acetate. Combined organic layer was washed with water and brine and dried over anhydrous sodium sulfate. The solvent was evaporated under reduced pressure and the residue was purified over a silica gel column using 20% ethyl acetate-hexane as eluent to yield the title compound (270 mg).

¹H NMR (300 MHz, CDCl₃): δ 7.18-7.26 (4H, m), 5.21 (1H, bs), 4.46 (1H, s), 4.30 (2H, bs), 4.09-4.12 (1H, d, 6 Hz), 3.69-3.73 (4H, m), 3.53-3.63 (6H, m), 3.42 (1H, bs), 1.48-1.66 (5H, m), 1.35 (3H, s), 1.25 (18H, bs), 0.86 (3H, m).

LCMS (m/e): 579.37 (M⁺+1)

### Step b: Synthesis of 1-O-Dodecyl-2,3-O-isopropylidene-6-deoxy-6-{[{(4-[2-hydroxy-2-oxo-ethyl]-phenyl)-amino}-carbonyl]-amino}-α-L-sorbofuranoside

Lithium hydroxide monohydrate (30 mg) was added to a solution of 1-O-Dodecyl-2,3-O-isopropylidene-6-deoxy-6-{[{(4-[2-methoxy-2-oxo-ethyl]-phenyl)-amino}-carbonyl]-amino}-α-L-sorbofuranoside (270 mg) obtained from step a of Example 19, in tetrahydrofuran (6 mL), methanol (2 mL) and distilled water (2 mL) at room temperature and stirred overnight. The reaction mixture was concentrated under reduced pressure, the residue was taken in distilled water and acidified with dilute aqueous sodium hydrogen solution. The aqueous layer was extracted with ethyl acetate and the combined organic layer was washed with water and brine and dried over anhydrous sodium sulfate. The solvent was evaporated under reduced pressure to yield the title compound (200 mg).

¹H NMR (300 MHz, D₂O): δ 7.28-7.31 (2H, d, 9 Hz), 7.11-7.13 (2H, d, 6 Hz), 4.32 (1H, s), 4.14 (1H, m), 3.99 (1H, s), 3.18-3.56 (8H, m), 1.47-1.49 (2H, m), 1.39 (3H, s), 1.29 (1H, s), 1.24 (18H, bs), 0.85 (3H, t, 6 Hz).

LCMS (m/e): 565.42 (M⁺+1)

Analogs of 1-O-Dodecyl-2,3-O-isopropylidene-6-deoxy-6-{[{(4-[2-hydroxy-2-oxo-ethyl]-phenyl)-amino}-carbonyl]-amino}-α-L-sorbofuranoside (Compound No. 44) described below can be prepared by replacing (4-phenoxycarbonylamino-phenyl)-acetic acid methyl ester with the appropriate esters, as applicable in each case.
1-O-Dodecyl-2,3-O-isopropylidene-6-deoxy-6-[({[2-(carboxymethyl)phenyl]amino}-carbonyl)amino]-α-L-sorbofuranoside (Compound No. 66), or
1-O-Dodecyl-2,3-O-isopropylidene-6-deoxy-6-[({[3-(carboxymethyl)phenyl]amino}-carbonyl)amino]-α-L-sorbofuranoside (Compound No. 67).

### Example 20

### Synthesis of 1-O-Dodecyl-2,3-O-isopropylidene-6-deoxy-6-(ethylsulfonyl)-amino-α-L-sorbofuranoside (Compound No. 41)

Triethylamine (0.036 mL, 0.258 mmol) and ethanesulfonyl chloride (0.032 mg, 0.258 mmol) was added to a solution of 1-O-Dodecyl-2,3-O-isopropylidene-6-deoxy-6-amino-α-L-sorbofuranoside (100 mg) obtained from step b of Example 13 in dichloromethane (5 mL) at 0° C. The reaction mixture was stirred for 3 hours at room temperature and then taken into distilled water and extracted with dichloromethane. The combined organic layer was washed with water and brine and dried over anhydrous sodium sulfate. The solvent was evaporated under reduced pressure and the residue was purified over silica gel column using 25% ethyl acetate-hexane as eluent to yield title compound (100 mg).

¹H NMR (300 MHz, CDCl₃): δ 4.54 (1H, bs), 4.42-4.37 (2H, m), 4.12-4.09 (1H, d, 9 Hz), 3.80-3.72 (1H, m), 3.57-3.39 (6H, m), 3.11-3.03 (2H, q, 9 Hz), 1.57-1.26 (26H, m), 0.88 (3H, t, 6 Hz).

LCMS (m/e): 480.39 (M⁺+1)

### Example 21

### Synthesis of 1-O-Dodecyl-2,3-O-isopropylidene-4-O-methyl-6-deoxy-6-{[(4-fluorophenyl)acetyl]amino}-α-L-sorbofaranoside (Compound No. 108) (RBx14599)

### Step a: Synthesis of 1-O-Dodecyl-2,3-O-isopropylidene-6-deoxy-6-aza-α-L-sorbofuranoside

Sodium azide (2.6 gm) was added to a solution of 1-O-Dodecyl-2,3-O-isopropylidene-6-tosyl-α-L-sorbofuranoside (4.4 gm) in dimethylformamide (30 ml) at room temperature and the reaction mixture was heated for about 10 hours at 110° C. The dimethylformamide was distilled out under reduced pressure, the residue was taken in distilled water and extracted with ethyl acetate. The combined organic layer was dried over anhydrous sodium sulfate. The solvent was evaporated under reduced pressure to yield the title compound (4 g).

### Step b: Synthesis of 1-O-Dodecyl-2,3-O-isopropylidene-4-O-methoxy-6-deoxy-6-aza-α-L-sorbofuranoside

Sodium hydride (9 mg) was added to a solution of 1-O-Dodecyl-2,3-O-isopropylidene-6-deoxy-6-aza-α-L-sorbofuranoside (130 mg) obtained from step a above in tetrahydrofuran (5 ml) at 0° C. and stirred for 20 mins. At the same temperature methyliodide (0.1 ml) was added and further stirred for about 1 hr at room temperature. The tetrahydrofuran was distilled out under reduced pressure; the residue was taken in distilled water and extracted with ethyl acetate. The combined organic layers was dried over anhydrous sodium sulfate. The solvent was evaporated under reduced pressure to yield the title compound (100 mg)

### Step c: Synthesis of 1-O-Dodecyl-2,3-O-isopropylidene-4-O-methoxy-6-deoxy-6-amino-α-L-sorbofuranoside

20% Pd/C (20 mg) was added to a solution of 1-O-Dodecyl-2,3-O-isopropylidene-4-O-methoxy-6-deoxy-6-aza-α-L-sorbofuranoside (100 mg) obtained from step b above in methanol (10 mL). The reaction mixture was shaken under hydrogen atmosphere, using a Parr apparatus at 60 psi for 2 hours at room temperature. The reaction mixture was filtered over celite and the filtrate was concentrated under reduced pressure and the residue was purified over a silica gel column using 10% methanol-dichloromethane as eluent to yield the title compound (55 mg).

### d. Synthesis of 1-O-Dodecyl-2,3-O-isopropylidene-4-O-methyl-6-deoxy-6-{[(4-fluorophenyl)acetyl]amino}-α-L-sorbofuranoside (Compound No. 108)

4-Fluorophenylaceticacid (21 mg), 1-hydroxybezotriazole (20 mg) and N-methylmorpholine (0.1 ml) was added to a solution of 1-O-Dodecyl-2,3-O-isopropylidene-4-O-methyl-6-deoxy-6-amino-α-L-sorbofuranoside (55 mg) obtained from step c above in dimethylformamide (5 mL) at 0° C. and after 20 minutes at the same temperature was added EDCI.HCl (28 mg). The reaction mixture was allowed to warm to room temperature and stirred for 12 hours. The reaction mixture was concentrated under reduced pressure the residue was taken in distilled water and extracted with ethyl acetate. The combined organic layers were dried over anhydrous sodium sulfate. The solvent was evaporated under reduced pressure and the residue was purified over preparative TLC (thickness 2 mm) using 40% ethylacetate-hexane as eluant to yield the title compound (35 mg).

¹H NMR (400 MHz, CDCl₃): δ 7.21-7.26 (m, 2H), 7.00-7.05 (m, 2H), 5.84 (bs, 1H, NH), 4.49 (s, 1H), 4.31-4.33 (m, 1H) 3.63-3.65 (m, 1H), 3.43-3.59 (m, 8H), 3.44 (s, 3H), 1.55-1.58 (m, 2H), 1.26-1.48 (m, 24H), 0.86-0.89 (m, 3H).

LCMS; (m/z), 538 (M+1), 560 (M+Na)

### Example 22

### Synthesis of 1-O-(2-butoxyethyl)-2,3-O-isopropylidene-6-deoxy-6-{[(4-fluorophenyl)acetyl]-amino}-α-L-sorbofuranoside (Compound No. 113)

### Step a: Synthesis of 1-O-(2-butoxyethyl)-2,3-O-isopropylidene6-deoxy-6-aza-α-L-sorbofuranoside

Sodium azide (1.4 gm) was added to a solution of 1-O-(2-butoxyethyl)-2,3-O-isopropylidene-6-tosyl-α-L-sorbofuranoside (2.0 gm) in dimethylformamide (20 ml) at room temperature and the reaction mixture was heated for about 12 hours at 110° C. The dimethylformamide was distilled out under reduced pressure, the residue was taken in distilled water and extracted with ethyl acetate. The combined organic layer was washed with water and brine and dried over anhydrous sodium sulfate. The solvent was evaporated under reduced pressure the residue thus obtained was purified over a silica gel column using 20% ethyl acetate-hexane as a eluant to yield the title compound (1.1 g)

### Step b: Synthesis of 1-O-(2-butoxyethyl)-2,3-O-isopropylidene-4-O-methoxy-6-deoxy-6-amino-α-L-sorbofuranoside

10% Pd/C (100 mg) was added to a solution of 1-O-(2-butoxyethyl)-2,3-O-isopropylidene6-deoxy-6-benzylamino-α-L-sorbofuranoside (1 g) obtained from step a above, in methanol (30 mL). The reaction mixture was shaken under hydrogen atmosphere using a Parr apparatus at 60 psi for 2 hours at room temperature. The reaction mixture was filtered over celite and the filtrate was concentrated under reduced pressure and purified over a silica gel column using 20% ethyl acetate-hexane as eluent to yield the title compound (800 mg).

### Step c: Synthesis of 1-O-(2-butoxyethyl)-2,3-O-isopropylidene-6-deoxy-6-{[(4-fluorophenyl)acetyl]-amino}-α-L-sorbofuranoside (Compound No. 113)

4-fluorophenylaceticacid (48 mg), 1-hydroxybezotriazole (47 mg) and N-methylmorpholine (0.05 ml) was added to a solution of 1-O-(2-butoxyethyl)-2,3-O-isopropylidene-6-deoxy-6-amino-α-L-sorbofuranoside (100 mg) obtained from step b above in dimethylformamide (5 mL) at 0° C. and after 20 minutes at the same temperature was added EDCI.HCl (66 mg). The reaction mixture was allowed to warm to room temperature and stirred for 12 hours. The reaction mixture was concentrated under reduced pressure the residue was taken in distilled water and extracted with ethyl acetate. The combined organic layer was washed with water and brine and dried over anhydrous sodium sulfate. The solvent was evaporated under reduced pressure and the residue was purified over preparative TLC (thickness 2 mm) using 50% ethyl acetate-hexane as eluant to yield the title compound (125 mg).

¹H NMR (400 MHz, CDCl₃): [symbol] m, 2H), 7.01-7.05 (m, 2H), 5.82 (bs, 1H, NH), 5.45 (s, 1H), 4.20-4.27 (m, 1H), 3.95-3.97 (m, 2H), 3.65-3.73 (m, 11H), 1.25-1.61 (m, 10H), 0.87-0.91 (m, 3H).

LCMS: (m/z), 456 (M+1), 478 (M+Na)

Analogs of 1-O-(2-butoxyethyl)-2,3-O-isopropylidene-6-deoxy-6-{[(4-fluorophenyl)acetyl]-amino}-α-L-sorbofuranoside (Compound No. 113) described below can be prepared by replacing 4-fluorophenylacetic acid with the appropriate acids, as applicable in each case.
1-O-(2-butoxyethyl)-2,3-O-isopropylidene-6-deoxy-6-{[(3,4-difluorophenyl)acetyl]amino}-α-L-sorbofuranose (Compound No. 109),
1-O-(2-butoxyethyl)-2,3-O-isopropylidene-6-deoxy-6-{[(3,4 dichlorophenyl)acetyl]amino}-α-L-sorbofuranose (Compound No. 110),
1-O-(2-butoxyethyl)-2,3-O-isopropylidene-6-deoxy-6-{[(4-methoxyphenyl)acetyl]amino}-α-L-sorbofuranose (Compound No. 111), or
1-O-(2-butoxyethyl)-2,3-O-isopropylidene-6-deoxy-6-{[(3-methoxyphenyl)acetyl]amino}-α-L-sorbofuranose (Compound No. 112).

### Scheme IV

### Example 23

### Synthesis of Hydrochloride salt of 1-O-Dodecyl-2,3-O-isopropylidene-6-deoxy-6-{1-[4-({4-methoxy-3-[5-{1-methyl-3-propyl-7-oxo-1,6-dihydro-pyrazolo[4,3-d]-pyrimidinyl}]-phenyl}-sulfonyl)-piperazinyl]}-α-L-sorbofuranoside (Compound No. 47)

### Step a: Synthesis of 1-O-Dodecyl-2,3-O-isopropylidene-6-deoxy-6-(4-benzylpiperazin-1-yl)-α-L-sorbofuranoside

A mixture of 1-O-Dodecyl-2,3-O-isopropylidene-6-tosyl-α-L-sorbofuranoside (prepared as described in U.S. Pat. No. 5,637,570) (500 mg) and 1-benzylpiperazine (206 mg) was heated with stirring for 7-8 hours. The reaction mixture was cooled and triturated with ether. A white solid precipitated, which was filtered and the filtrate was washed with water, saturated aqueous sodium bicarbonate solution and brine. The organic layer was dried over anhydrous sodium sulfate and concentrated in vacuo. The residue was purified over a silica gel column using 30% ethyl acetate-hexane as eluent to yield the title compound (550 mg).

### Step b: Synthesis of 1-O-Dodecyl-2,3-O-isopropylidene-6-deoxy-6-(piperazin-1-yl)-α-L-sorbofuranoside

10% Palladium/carbon (275 mg) and ammonium formate (1.0 g) was added with stirring to solution of the 1-O-Dodecyl-2,3-O-isopropylidene-6-deoxy-6-(4-benzylpiperazin-1-yl)-α-L-sorbofuranoside (550 mg) obtained from step a above in methanol (20 mL) and the reaction mixture was refluxed. After 1 hour, the reaction mixture was cooled and filtered through a bed of celite. It was repeatedly washed with hot dichloromethane and then washed with water. The organic layer was dried over anhydrous sodium sulfate and concentrated in vacuo. The residue thus obtained was purified using column chromatography using 2.5% methanol-dichloromethane as eluent to furnish the title compound (450 mg).

### Step c: Synthesis of 1-O-Dodecyl-2,3-O-isopropylidene-6-deoxy-6-{1-[4-({4-methoxy-3-[5-{1-methyl-3-propyl-7-oxo-1,6-dihydro-pyrazolo[4,3-d]-pyrimidinyl}]-phenyl}-sulfonyl)-piperazinyl]}-α-L-sorbofuranoside

4-methoxy-3-(1-methyl-7-oxo-3-propyl-6,7-dihydro-1H-pyrazolo[4,3-d]pyrimidin-5-yl)-benzenesulfonyl chloride was mixed with 1-O-Dodecyl-2,3-O-isopropylidene-6-deoxy-6-(piperazin-1-yl)-α-L-sorbofuranoside (488 mg) obtained in step b above and dissolved in a dichloromethane:pyridine (5:1 mL) mixture at 0° C. After 1 hour, the solvent mixture was removed under reduced pressure and the residue was taken in dichloromethane. The organic layer was washed with saturated aqueous sodium bicarbonate solution and water and then dried over anhydrous sodium sulfate. The solvent was evaporated under reduced pressure and the residue was purified over a silica gel column using 2% methanol-dichloromethane as eluent to yield the title compound (350 mg).

¹H NMR (CDCl₃, 300 MHz): δ 8.82 (1H, d, 2 Hz), 7.81 (1H, dd, 2&6.6 Hz), 7.15 (1H, d, 8.7 Hz), 4.78 (1H, bs), 4.37 (4H, m), 4.27 (5H, s), 4.1 (1H, s), 3.61 (1H, m), 3.55-3.48 (3H, m), 3.08 (4H, bs), 2.92 (2H, t, 7.4 Hz), 2.85 (2H, m), 2.8-2.1 (7H, m), 1.85 (2H, q, 7.4 Hz), 1.68 (3H, m), 1.44 (3H, s), 1.31-1.24 (2.4H, m), 1.02 (3H, m), 0.87 (3H, t, 5.8 Hz).

### Step d: Synthesis of Hydrochloride salt of 1-O-Dodecyl-2,3-O-isopropylidene-6-deoxy-6-{1-[4-({4-methoxy-3-[5-{1-methyl-3-propyl-7-oxo-1,6-dihydro-pyrazolo[4,3-d]-pyrimidinyl}]-phenyl}-sulfonyl)-piperazinyl]}-oz-L-sorbofuranoside

Ethereal HCl was added to 1-O-Dodecyl-2,3-O-isopropylidene-6-deoxy-6-{1-[4-({4-methoxy-3-[5-{1-methyl-3-propyl-7-oxo-1,6-dihydro-pyrazolo[4,3-d]-pyrimidinyl}]-phenyl}-sulfonyl)-piperazinyl]}-α-L-sorbofuranoside (100 mg) obtained from step c above, dissolved in minimum amount of ether (5 mL) at 0° C. and stirred for about 40 minutes. The volatiles were removed under reduced pressure and the residue triturated with hexane to yield a white solid that was filtered and dried in vacuo to produce the title compound (100 mg).

### Scheme V

### Example 24

### Synthesis of 1-O-[(4-nitro-phenyl-amino-carbonyloxy)-hexyl]-2,3;4,6-di-O-isopropylidene-α-L-sorbofuranoside (Compound No. 48)

### Step a: Synthesis of 1-O-[(6-hydroxyhexyl]-2,3;4,6-di-O-isopropylidene-α-L-sorbofuranoside

Potassium hydroxide (12.9 g), followed by 6-chloro-1-hexanol (11.56 g) and a pinch of tetrabutylammonium iodide was added to a solution of a 2,3;4,6-di-O-isopropylidene-α-L-sorbofuranoside (20 g)(prepared as described in U.S. Pat. No. 6,329,344) in dimethylsulfoxide (80 mL) at room temperature. The reaction mixture was stirred at 50° C. and after 5 hours, the reaction mixture was cooled and water was added. The aqueous layer was extracted with ethyl acetate and the organic extract was washed with water and brine, and dried over anhydrous sodium sulfate. The solvent was removed under reduced pressure and product was purified over a silica gel column using 10% ethyl acetate-hexane as eluent to yield the title compound (22.5 g).

### Step b: Synthesis of 1-O-[(4-nitro-phenyl-amino-carbonyloxy)-hexyl]-2,3;4,6-di-O-isopropylidene-α-L-sorbofuranoside

P-nitrophenyl isocyanate (0.51 g) was added to a solution of 1-O-[(6-hydroxyhexyl]-2,3;4,6-di-O-isopropylidene-α-L-sorbofuranoside obtained from step a of Example 22 (1.0 g) in dichloromethane (3 mL) at 0° C. with continuous stirring. The reaction mixture was warmed to room temperature and stirred for 24 hours. The solvent was removed at reduced pressure and the residue was purified by column chromatography using 20% ethyl acetate-hexane as eluent to furnish the title compound (1.24 g)

¹HNMR (CDCl₃, 300 MHz): δ 8.20 (2H, d, 8.9 Hz), 7.57 (2H, d, 8.9 Hz), 4.46 (1H, s), 4.30 (1H, s), 4.20 (2H, t, 6.2 Hz), 4.09-4.02 (3H, m), 3.77-3.39 (4H, m), 1.69-1.23 (20H, m)

LCMS (m/e): 542.0 (M+NH₄), 547 (M+Na)

Analogs of 1-O-[(4-nitro-phenyl-amino-carbonyloxy)-hexyl]-2,3;4,6-di-O-isopropylidene-α-L-sorbofuranoside (Compound No. 48) described below can be prepared by replacing p-nitrophenyl isocyanate with the appropriate isocyanate, as applicable in each case.
1-O-[6-{(4-Chloro-phenyl)-amino-carbonyloxy}-hexyl]-2,3;4,6-di-O-isopropylidene-α-L-sorbofuranoside (Compound No. 49),
1-O-[6-{(4-Methoxy-phenyl)-amino-carbonyloxy}-hexyl]-2,3;4,6-di-O-isopropylidene-α-L-sorbofuranoside (Compound No. 50),
1-O-{6-[(4-Methyl-phenyl)-amino-carbonyloxy]-hexyl}-2,3;4,6-di-O-isopropylidene-α-L-sorbofuranoside (Compound No. 51).

### Scheme VI

### Example 25

### Synthesis of (4ξ)-1-O-Dodecyl-2,3-O-isopropylidene-4-C-methyl-4-O-{[(phenyl sulfonyl)-amino]-carbonyl}-6-deoxy-α-L-erythro-hex-2-ulofuranoside (Compound No. 53)

### Step a: Synthesis of (4ξ)-1-O-Dodecyl-2,3-O-isopropylidene-4-C-methyl-6-deoxy-α-L-erythro-hex-2-ulofuranoside

Methyl magnesium chloride in tetrahydrofuran (100 mL) was added to a solution of (4ξ)-1-O-Dodecyl-2,3-O-isopropylidene-4-oxo-6-deoxy-α-L-erythro-hex-2-ulofuranoside (2.60 g) prepared in step a of Example 6 at 0° C. in tetrahydrofuran (100 mL). The reaction mixture was warmed to room temperature and stirred for 2 hrs. The reaction mixture then was quenched with water (5 mL) and concentrated. The reaction mixture was extracted with ethyl acetate and the organic extracts were washed with water, brine and dried over sodium sulfate. The solvent was evaporated and the residue purified over a silica gel column using 5% ethyl acetate-hexane as eluent to yield the title compound as viscous oil (1.93 g).

¹H NMR (CDCl₃): δ 4.12 (s, 1H), 3.91 (q, J=6.0 Hz, 1H), 3.57-3.46 (m, 4H), 2.64 (s, 1H, —OH), 1.57 (s, 6H), 1.41 (s, 3H), 1.26-1.15 (m, 2H), 0.88 (t, J=6.0 Hz, 3H).

LCMS (m/e): 403 (M⁺+18).

### Step b: Synthesis of (4ξ)-1-O-Dodecyl-2,3-O-isopropylidene-4-C-methyl-4-O-{[(phenyl sulfonyl)-amino]-carbonyl}-6-deoxy-α-L-erythro-hex-2-ulofuranoside

Benzenesulfonyl isocyanate (0.13 mL) was added slowly to a stirred solution of (4ξ)-1-O-Dodecyl-2,3-O-isopropylidene-4-C-methyl-6-deoxy-α-L-erythro-hex-2-ulofuranoside (0.12 g) obtained from step a above in dichloromethane (2 mL) and refluxed for 12 hours. The reaction mixture was concentrated and residue purified over a silica gel column to yield the title compound as pale brown solid (0.17 g).

¹H NMR (CDCl₃) δ: 8.00 (d, J=6.0 Hz, 2H), 7.62 (m, 1H), 7.54 (m, 2H), 4.57 (s, 1H), 4.00 (q, J=6.0 Hz, 1H), 3.50-3.39 (m, 4H), 1.52 (d, J=6.0 Hz, 3H), 1.34-1.17 (m, 29H), 0.88 (t, J=6.0 Hz, 3H);

LCMS (m/e): 587 (M⁺+18).

Analogs of (4ξ)-1-O-Dodecyl-2,3-O-isopropylidene-4-C-methyl-4-O-{[(phenyl sulfonyl)-amino]-carbonyl}-6-deoxy-α-L-erythro-hex-2-ulofuranoside (Compound No. 53) described below can be prepared by replacing sulfonyl isocyanate with the appropriate isocyanate, as applicable in each case.
(4ξ)-1-O-Dodecyl-2,3-O-isopropylidene-4-C-methyl-4-O-[{[(4-methyl-phenyl)-sulfonyl]-amino}-carbonyl]-6-deoxy-α-L-erythro-hex-2-ulofuranoside (Compound No. 54),
(4ξ)-1-O-Dodecyl-2,3-O-isopropylidene-4-C-methyl-4-O-[{[(4-chloro-phenyl)-sulfonyl]-amino}-carbonyl]-6-deoxy-α-L-erythro-hex-2-ulofuranose (Compound No. 55),
(4ξ)-1-O-Dodecyl-2,3-O-isopropylidene-4-C-methyl-4-O-{[{2,5-dichloro-phenyl)-sulfonyl}-amino]-carbonyl}-6-deoxy-α-L-erythro-hex-2-ulofuranose (Compound No. 56),
(4ξ)-1-O-Dodecyl-2,3-O-isopropylidene-4-C-methyl-4-[{[(2-methyl-phenyl)-sulfonyl]-amino}-carbonyl]-6-deoxy-α-L-erythro-hex-2-ulofuranose (Compound No. 57),
(4ξ)-1-O-Dodecyl-2,3-O-isopropylidene-4-C-heptyl-4-O-[2-(1-piperidinyl)-ethyl]-6-deoxy-α-L-erythro-hex-2-ulofuranoside (Compound No. 58),
(4ξ)-1-O-Dodecyl-2,3-O-isopropylidene-4-C-heptyl-4-O-[2-(1-azepanyl)-ethyl]-6-deoxy-α-L-erythro-hex-2-ulofuranoside (Compound No. 59),
(4ξ)-1-O-Dodecyl-2,3-O-isopropylidene-4-C-heptyl-4-O-[2-(1-morpholinyl)-ethyl]-6-deoxy-α-L-erythro-hex-2-ulofuranoside (Compound No. 60),
(4ξ)-1-O-Dodecyl-2,3-O-isopropylidene-4-C-heptyl-4-O-[2-(1-pyrrolidinyl)-ethyl]-6-deoxy-α-L-erythro-hex-2-ulofuranoside (Compound No. 61),
(4ξ)-1-O-Dodecyl-2,3-O-isopropylidene-4-C-heptyl-4-O-heptyl-6-deoxy-α-L-erythro-hex-2-ulofuranoside (Compound No. 62),
(4ξ)-1-O-Dodecyl-2,3-O-isopropylidene-4-C-heptyl-4-O-[2-(1-dimethylamino)-propyl]-6-deoxy-α-L-erythro-hex-2-ulofuranoside (Compound No. 63),
(4ξ)-1-O-Dodecyl-2,3-O-isopropylidene-4-C-methyl-4-O-[{[(2-azepanyl)-sulfonyl]-amino}-carbonyl]-6-deoxy-α-L-erythro-hex-2-ulofuranoside (Compound No. 64).

### Scheme VII

### Example 26

### Synthesis of 1-O-Heptyl-2,3-O-isopropylidene-6-O-{[(4-meth 1-phenyl)-amino]-carbonyl}-α-L-sorbofuranoside (Compound No. 65)

P-methylphenyl isocyanate (0.08 mL) was added to a solution of 1-O-Heptyl-2,3-O-isopropylidene-α-L-sorbofuranoside (100 mg) in dichloromethane (10 mL) at room temperature and stirred overnight at room temperature. The reaction mixture was quenched with water and extracted with dichloromethane. The combined organic layer was washed with water and brine and dried over anhydrous sodium sulfate. The solvent was evaporated under reduced pressure and the residue was purified over silica gel column using 30% ethyl acetate-hexane as eluent to yield the title compound (130 mg).

¹H NMR (300 MHz, CDCl₃): δ 7.26-7.09 (4H, m), 6.64 (1H, s), 4.51 (2H, m), 4.44 (1H, s), 4.13-4.12 (2H, m), 3.85-3.76 (2H, dd, J=15 Hz), 3.60-3.53 (3H, m), 2.31 (3H, s), 1.58 (3H, s), 1.52 (3H, s), 1.34-1.25 (10H, m), 0.86 (3H, s).

LCMS (m/e): 474 (M⁺+Na).

### Pharmacological Activity

The compounds of the present invention are tested in one or more of the assays described herein. Standard assays are used to evaluate activity of compounds in present invention on inflammatory cells. Attenuation of agonist-induced release of lipid mediator of neutrophil chemotaxis, leukotriene B4 (LTB4), is used to evaluate inhibitory effect on neutrophils.

### A23187 Induced LTB₄ Release

Venous blood was collected from healthy human donors using heparin as an anti-coagulant. Neutrophils were isolated from freshly drawn blood after dextran sedimentation and ficoll separation (Eur J. Biochem. 169, 175, 1987). 180 μl of the of neutrophil suspension (0.2×10⁶ cells/ml) was taken and added 19 μL of Hank's Buffer salt solution along with 1 μL of the test drug (200 times concentrated) in a 24 well plate and incubated at 37° C. for 1 hour. 3 minutes before the end of test compound incubation, 0.25 mM Ca⁺⁺/Mg⁺⁺ were added. Then, 0.3 μg/ml A23187 (Sigma Chem, USA) was added and incubated for further 10 min at 37° C. The reaction was stopped by adding 80 μL of cold methanol and centrifuged to remove cell debris (J Pharmacol Exp Ther. 297:267, 2001). The samples were analysed for LTB₄ release using LTB₄ ELISA kits (Assay Design Inc., USA). The amount of LTB₄ released was quantified and percent inhibition of LTB₄ release was calculated with respect to the difference between the A23187 stimulated and negative control cells, to compute IC₅₀ values. In vitro data obtained on the compounds 1, 22-25, 28-46, 52-57, and 65-118 showed IC₅₀ values of from >30 μM to about 1.3 μM, for example, from about 25 μM to about 1.3 μM, for example, from about 10 μM to about 1.3 μM, for example, from about 3 μM to about 1.3 μM.

### Assay for 5-Lipoxygenase Activity

In a 96 well UV-plate, 100 μl of phosphate buffer saline (PBS) containing DTT (200 μM), ATP (100 μM) and calcium chloride (100 μM) was added. To each well 0.5 μl of test drug (200 times concentrated) or vehicle was added, followed by 4 μl of recombinant 5-Lox (3 units/μl) and was incubated at 37° C. for 5 min. The reaction was initiated by adding 1 μl of 1 mM freshly prepared arachidonic acid and increase in absorbance was monitored at 236 nm for 10 min. (J. Biol. Chem. 261:11512, 1986). A plot of absorbance verses time curve was prepared and area under curve (AUC) was computed for each well. Percent inhibition of AUC for different treatments was calculated with respect to the difference between the Arachidonic acid stimulated and negative control values, to compute IC₅₀ values. Compounds 69, 70, 78, 94, 106 and 116-118 were examined, giving IC₅₀ values of from about 5.4 μM to about 0.10 μM, for example, from about 1.7 μM to about 0.10 μM, for example, from about 0.75 μM to about 0.10 μM, for example, from about 0.30 to about 0.10 μM.

1. A compound having a structure of Formula I, 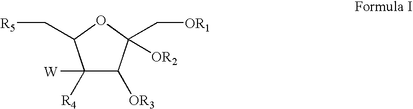
wherein
W is hydrogen or alkyl;
R₁ is allyl, alkenyl, alkynyl, cycloalkyl, aryl, heterocyclyl, heteroaryl, heterocyclylalkyl, heteroarylalkyl, aralkyl or —(CH₂)ₙO(C═O)NHRₓ,
wherein n is an integer 2-10, and
Rₓ is allyl, alkenyl, alkynyl, cycloalkyl, aryl, aralkyl, heteroaryl, heterocyclyl, heterocyclylalkyl or heteroarylalkyl;
R₂ ad R₃ together form a five-membered acetal, wherein the carbon joining the two oxygen atoms is substituted with Rₗ and Rₘ,
wherein Rₗ and Rₘ is hydrogen, alkyl, alkenyl, alkynyl, cycloalkyl, aryl or aralkyl; Rₗ and Rₘ together join to form a cyclic ring; or Rₗ and Rₘ together join to form an oxo,
wherein the ring optionally contains one or more heteroatoms selected from O, N or S, and the ring optionally is substituted with one or more of alkyl, alkenyl, alkynyl, acyl, substituted amino, cycloalkyl, carboxy, oxo, hydroxy alkoxy, aryloxy, halogen, aryl, aralkyl, heteroaryl, heterocyclyl, heteroarylalkyl, heterocyclylalkyl, or C(═O)QR₇,
wherein Q is O or NH, and R₇ is alkyl, alkenyl, alkynyl, aryl, aralkyl, cycloalkyl, or heteroarylalkyl; or when Q is NH, R₇ also is heteroaryl, heterocyclyl or heterocyclylalkyl; or
R₂ and R₃, instead of forming an acetal, optionally and independently is lower (C₁-C₄)-alkyl, (CH₂)ₖ-aryl, —C(═R_{y})NHRₓ or acyl,
wherein k is an integer from 1-4,
R_{y} is O or S, and
Rₓ is alkyl, alkenyl, alkynyl, cycloalkyl, aryl, aralkyl, heteroaryl, heterocyclyl, heterocyclylalkyl or heteroarylalkyl; and
R₅ is hydrogen, alkyl, cycloalkyl, heteroaryl, heterocyclyl, —NRₚRⱼ, or OR_{z}; or when R₄ is (wherein w, Rₐ, R_{b} and R_{c} same as defined earlier);
Rₚ and Rⱼ independently is hydrogen, alkyl, cycloalkyl, aryl, heteroaryl, heterocyclyl, heteroarylalkyl, heterocyclylalkyl, or aralkyl, or Rₚ and Rⱼ together join to form a cyclic ring, which optionally is benzofused, containing 0-4 heteroatom selected from 0-4 heteroatoms selected from O, S or N wherein the ring is substituted with one or more of alkyl, alkenyl, alkynyl, amino, substituted amino, cycloalkyl, carboxy, oxo, hydroxy, alkoxy, aryloxy, halogen, aryl, aralkyl, heteroaryl, heterocyclyl, heteroarylalkyl or heterocyclylalkyl;
wherein R_{z} is alkyl, alkenyl, alkynyl, cycloalkyl, aryl, aralkyl, heteroaryl, heterocyclyl, heteroarylalkyl, heterocyclylalkyl, acyl or —C(═O)NR_{f}R_{q},
wherein R_{f} and R_{q} are each independently hydrogen, alkyl, alkenyl, alkynyl, cycloalkyl, aryl, aralkyl, heterocyclyl, heteroaryl, heteroarylalkyl, heterocyclylalkyl or S(O)₂R₆; or R_{f} and R_{q} together form a ring,
wherein R₆ is alkyl, alkenyl, alkynyl, cycloalkyl, aryl, aralkyl, heteroaryl, heterocyclyl, heterocyclyalkyl, heteroaryl alkyl or substituted amino;
Rₛ is alkyl, alkenyl, alkynyl, cycloalkyl, aryl, aralkyl, heterocyclylalkyl or heteroarylalkyl,
Y is —C(═O), —C(═S) or SO₂),
R_{d} is alkyl, alkenyl, alkynyl, cycloalkyl, aryl, aralkyl, heteroaryl, heterocyclyl, heteroarylalkyl or heterocyclylalkyl,
T is O, S, —N(CN), —N(NO₂), or —CH(NO₂),
Rₜ is H, OH or Rₓ,
Rₓ is alkyl alkenyl, alkynyl, cycloalkyl, aryl, aralkyl, heteroaryl, heterocyclyl, heterocyclylalkyl or heteroarylalkyl,
w is 1-4, and
Rₐ and R_{b} are each independently hydrogen or R_{d}, or Rₐ and R_{b}, together with the nitrogen atom carrying them, is the N-terminus of an amino acid or di-tetrapeptide,
wherein R_{f} and R_{d} are each independently is hydrogen, alkyl, alkenyl, alkynyl cycloalkyl, aryl, aralkyl, heterocyclyl, heteroaryl, heteroarylalkyl, heterocyclylalkyl or S(O)₂R₆; or R_{f} and R_{q} together form a ring,
wherein R_{d} is alkyl, alkenyl, alkynyl, cycloalkyl, aryl, aralkyl, heteroaryl, heterocyclyl, heterocyclylalkyl, heteroarylalkyl or substituted amino;
and wherein when W is hydrogen, then R₄ is hydrogen, OR_{c}, —NHC(═O)OR₅, —NHYR_{d}, —NHC(=T)NRₜRₓ, or —(CH₂)_{w}(C═O)NRₐR_{b},
wherein R_{c} is hydrogen, alkyl, cycloalkyl, aryl, heteroaryl, heterocyclyl, acyl or —C(═O)NR_{f}R_{q},
Rₛ is alkyl, alkynyl, alkynyl, cycloalkyl, aryl, aralkyl, heterocyclylalkyl or heteroarylalkyl,
Y is —C(═O ), —C(═S) or SO₂),
R_{d} is alkyl, alkenyl, alkynyl, cycloalkyl, aryl, aralkyl, heteroaryl, heterocyclyl, heteroarylalkyl or heterocyclylalkyl,
T is O, S, —N(CN), —N(NO₂), or —CH(NO₂),
Rₜ is H, OH or Rₓ,
Rₓ is alkyl, alkenyl, alkynyl, cycloalkyl, aryl, aralkyl, heteroaryl, heterocyclyl, heterocyclylalkyl or heteroarylalkyl,
w is 1-4, and
Rₐ and R_{b} are each independently hydrogen or R_{d}, or Rₐ and R_{b}, together with the nitrogen atom carrying them, can be the N-terminus of an amino acid or di-tetrapeptide,
wherein R_{f} and R_{q} are each independently hydrogen, alkyl, alkenyl, alkynyl, cycloalkyl, aryl, aralkyl, heterocyclyl, heteroaryl, heteroarylalkyl, heterocyclylalkyl or S(O)₂R₆; or R_{f} and R_{q} together form a ring,
wherein R₆ is alkyl, alkenyl, alkynyl, cycloalkyl, aryl, aralkyl, heteroaryl, heterocyclyl, heterocyclylalkyl, heteroarylalkyl or substituted ammo; or
when W is alkyl, then R₄ is —OR_{z},
wherein R_{z} is alkyl, alkenyl, alkynyl, cycloalkyl, aryl, aralkyl, heteroaryl, heterocyclyl, heteroarylalkyl, heterocyclylalkyl, acyl or —C(═O)NR_{f}R_{q},
wherein R_{f} and R_{q} are each independently hydrogen, alkyl, alkenyl, alkynyl, cycloalkyl, aryl, aralkyl, heterocyclyl, heteroaryl, heteroarylalkyl, heterocyclylalkyl or S(O)₂R₆; or R_{f} and R_{q} together form a ring,
wherein R₆ is alkyl, alkenyl, alkynyl, cycloalkyl, aryl, aralkyl, heteroaryl, heterocyclyl, heterocyclylalkyl, heteroarylalkyl or substituted amino.

2. The compound of claim 1, wherein R₁ is alkyl or —(CH₂)ₙO(C═O)NHRₓ.

3-4. (canceled)

5. The compound of claim 1, wherein A wherein R₄ is OH, OR_{c} or H, then R₅ is NHYR_{d} or —NHC(=T)NRₜRₓ.

6. The compound of claim 1, wherein W is hydrogen, then R₄ is hydrogen, OR_{c}, —NHYR_{d}, or —NHC(=T)NRₜRₓ,

7-8. (canceled)

9. The compound of claim 1, wherein Rₗ and Rₘ joins a cyclic ring and the cyclic ring optionally is substituted with one or more of alkyl, alkenyl, alkynyl, acyl, substituted amino, cycloalkyl, carboxy, oxo, hydroxy, alkoxy, aryloxy, halogen, aryl, aralkyl, heteroaryl, heterocyclyl, heteroarylalkyl, heterocyclylalkyl, or —C(═O)QR₇.

10. (canceled)

11. A compound, wherein the compound is:
1-O-decyl-2,3-O-isopropylidene-4-O-{[(phenyl-sulfonyl)-amino]-carbonyl}-6-deoxy-α-L-sorbofuranoside (Compound No. 1);
1-O-Dodecyl-2,3-O-isopropylidene-4-O-{[(4-chloro-phenyl)-sulfonylamino]carbonyl}-6-deoxy-6-(1-azepanyl)-α-1-sorhofuratioside (Compound No. 2);
1-O-Dodecyl-2,3-O-isopropylidene-4-O-{[phenyl-sulfonylamimo]-carbonyl}-6-deoxy-6-(1-azepanyl)-α-L-sorbofuranoside (Compound No. 3);
1-O-Dodecyl-2,3-O-isopropylidene-4-O-{[(4-methyl-phenyl)-sulfonylamino]-carbonyl)}-6-deoxy-6-(1-azepanyl-1)-α-L-sorbofuranoside (Compound No. 4);
1-O-Dodecyl-2,3-O-isopropylidene-4-O-{[(4-methyl-phenyl)-sulfonylamino]-carbonyl}-6-deoxy-6-(4-morpholinyl)-α-L-sorbofuranoside (Compound No. 5);
1-O-Dodecyl-2,3-O-isopropylidene-4-O-{[(4-chloro-phenyl)-sulfonylamino]carbonyl}-6-deoxy-6-(4-morpholinyl)-α-L-sorbofuranoside (Compound No. 6);
1-O-Dodecyl-2,3-O-isopropylidene-4-O-{[(phenyl-sulfonyl)-amino]carbonyl}-6-deoxy-6-(4-morpholinyl)-α-L-sorbofuranoside (Compound No. 7);
1-O-Dodecyl-2,3-O-isopropylidene-4-O-{[(phenylsulfonyl)-amino]-carbonyl}-6-deoxy-6-(1-pyrrolidinyl)-α-L-sorbofuranoside (Compound No. 8);
1-O-Dodecyl-2,3-O-isopropylidene-4-O-{[(4-methyl-phenyl)-sulfonylamino]-carbonyl}-6-deoxy-α-sorbofuranoside (Compound No. 9);
Hydrochloride salt of 1-O-Dodecyl-2,3-O-isopropylidene-4-O-{[(4-methyl-phenyl)-sulfonylamino]-carbonyl}-6-deoxy-6-(1-pyrrolidyl)-α-L-sorbofuranoside (Compound No. 10);
Hydrochloride salt of 1-O-Dodecyl-2,3-O-isopropylidene-4-O-{[(4-chloro-phenyl)-sulfonylamino]-carbonyl}-6-deoxy-6-(1-pyrrolidinyl)-α-L-sorbofuranoside (Compound No. 11);
1-O-Heptyl-2,3-O-isopropylidene-4-O-{[4-(2-methoxy-2-oxo-ethyl)-phenyl]-amino}-carbonyl-6-deoxy-6-(1-azepanyl)-α-sorbofuranoside (Compound No. 12);
1-O-Heptyl-2,3-O-isopropylidene-4-O-{[4-(2-methoxy-2-oxo-ethyl)-phenyl]-amino}carbonyl-6-deoxy-6-(4-morpholinyl)-α-L-sorbofuranoside (Compound No. 13);
Hydrochloride salt of 1-O-heptyl-2,3-O-isopropylidene-4-O-{[4-(2-methoxy-2-oxo-ethyl)-phenyl]-amino}-carbonyl-6-deoxy-6-(1-azepanyl)-α-L-sorbofuranoside (Compound No. 14);
1-O-Dodecyl-2,3-O-isopropylidene-4-O-{[4-(2-methoxy-2-oxo-ethyl)-phenyl]-amino}-carbonyl-6-deoxy-6-(1-azepanyl)-α-L-sorbofuranoside (Compound No. 15);
1-O-Dodecyl-2,3-O-isopropylidene-4-O-{[4-(2f-ethoxy-2-oxo-ethyl)-phenyl]-amino}-carbonyl-6-deoxy-6-(4-morpholinyl)-α-L-sorbofuranoside (Compound No. 16);
1-O-Dodecyl-2,3-O-isopropylidene-4-O-{[4-(2-methoxy-2-oxo-ethyl)-phenyl]-amino}carbonyl-6-deoxy-6-(1-pyrrolidinyl)-α-L-sorbofuranoside (Compound No. 17);
1-O-Dodecyl-2,3-O-isopropylidene-4-O-{[4-(2-methoxy-2-oxo-ethyl)-phenyl]-amino}-carbonyl-6-deoxy-6-(1-piperidinyl)-α-L-sorbofuranoside (Compound No 18);
1-O-Heptyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 19);
1-O-Decyl-2,3-O-isopropylidene-4-O-{[(4-[2-hydroxy-2-oxo-ethyl]-phenyl)-amino]-carbonyl}-6-deoxy-6-(1-azepanyl)-α-L-sorbofuranoside (Compound No. 20);
Tris salt of 1-O-Heptyl-2,3-O-isopropylidene-4-O-{[4-(2-hydroxy-2-oxo-ethyl)-phenyl]-amino}-1-carbonyl-6-deoxy-6-(1-azepanyl)-α-L-sorbofuranoside (Compound No. 21);
Tris salt of-1-O-decyl-2,3-O-isopropylidene-4-O-{[4-(2-hydroxy-2-oxo-ethyl-phenyl]-amino}-carbonyl-6-deoxy-6-(1-azepanyl)-α-L-sorbofuranoside (Compound No. 22);
1-O-Dodecyl-2,3-O-isopropylidene-4-O-{[4-(2-hydroxy-2-oxo-ethyl)-phenyl]-amino}-carbonyl-6-deoxy-6-(1-piperidinyl)-α-L-sorbofuranoside (Compound No. 23);
1-O-Dodecyl-2,3-O-isopropylidene-4-O-{[4-(2-hydroxy-2-oxo-ethyl)-phenyl]-amino}-carbonyl-6-deoxy-6-(4-morpholinyl)-α-L-sorbofuranoside (Compound No. 24);
1-O-Dodecyl-2,3-O-isopropylidene-4-O-{[4-(2-hydroxy-2-oxo-ethyl)-phenyl]-amino}-carbonyl-6-deoxy-6-(1-azepanyl)-α-L-sorbofuranoside (Compound No. 25);
1-O-Dodecyl-2,3-O-isopropylidene-4-O-{[4-(2-hydroxy-2-oxo-ethyl)-phenyl]-amino}-carbonyl-6-deoxy-6-(1-pyrrolidinyl)-α-L-sorbofuranoside (Compound No. 26);
1-O-Dodecyl-2,3-O-isopropylidene-4-O-[4-(2-hydroxy-2-oxo-phenyl)-amino]-carbonyl-6-deoxy-6-(1-azepanyl)-α-L-sorbofuranoside (Compound No. 27);
Tris salt of 1-O-Decyl-2,3-O-isopropylidene-4-O-[(4-{2-hydroxy-2-oxo-ethyl}-phenyl)-amino]-carbonyl-6-deoxy-6-[2-(1-pyrrolidinyl)-ethyl]-amino-α-L-sorbofuranoside (Compound No. 28);
Tris salt of 1-O-Decyl-2,3-O-isopropylidene-4-O-[{4-(2-hydroxy-2-oxo-ethyl)-phenyl}-amino]-carbonyl-6-deoxy-6-[2-(1-piperidinyl)-ethyl]amino-α-L-sorbofuranoside. (Compound No. 29);
Tris salt of 1-O-Decyl-2,3-O-isopropylidene-4-O-{[4-(2-hydroxy-2-oxo-ethyl)-phenyl]-amino}-carbonyl-6-deoxy-6-[2-(4-morpholinyl)-ethyl]-amino-α-L-sorbofuranoside (Compound No. 30);
Tris salt of 1-O-Decyl-2,3-O-isopropylidene-4-O-{[4-(2-hydroxy-2-oxo-ethyl)-phenyl]-amino}-carbonyl-6-deoxy-6-[2-(1-cycloheptyl-amino)-ethyl]-amino-α-L-sorbofuranoside (Compound No. 31);
(4ξ)-1-O-Dodecyl-2,3-O-isopropylidene-4,6-dideoxy-4-[{[(4-fluoro-phenyl)-amino]-carbonyl}-amino]-α-L-erythro-hex-2-ulofuranoside (Compound No. 32);
(4ξ)-1-O-Dodecyl-2,3-O-isopropylidene-4,6-dideoxy-4-{[(butyl-amino)-carbonyl]-amino}-α-L-erythro-hex-2-ulofuranoside (Compound No. 33);
(4ξ)-1-O-Dodecyl-2,3-O-isopropylidene-4,6-dideoxy-4-([(4-fluoro-phenyl)-sulfonyl]amino)-α-L-erythro-hex-2-ulofuranoside (Compound No. 34);
(4ξ)-1-O-Dodecyl-2,3-O-isopropylidene-4,6-dideoxy-4-{[(4-fluoro-phenyl)-carbonyl]-amino}-α-1-erythro-hex-2-ulofuranoside (Compound No. 35);
(4ξ)-1-O-Dodecyl-2,3-O-isopropylidene-4,6-dideoxy-4-[{[(2-phenylethyl)-amino]-thiocarbonyl}-amino]-α-L-erythro-hex-2-ulofuranoside (Compound. No. 36);
(4ξ)-1-O-Dodecyl-2,3-O-isopropylidene-4,6-dideoxy-4-[{[(4-[2-hydroxy-2-oxo-ethyl]-phenyl)-amino]-carbonyl}-amino]-α-L-erythro-hex-2-ulofuranoside (Compound No. 37);
(4ξ)-1-Dodecyl-2,3-O-isopropylidene-4,6-dideoxy-4-{[3-(1,3-benzodionol-5-yl)-propanoyl]-amino}-α-L-erythro-hex-2-ulofuranoside (Compound No. 31);
1-O-Dodecyl-2,3-O-isopropylidene-6-deoxy-6-{[(4-fluorophenyl)-amino]-carbonyl}-amino-α-L-sorbofuranoside (Compound No. 39);
1-O-Dodecyl-2,3-O-isopropylidene-6-deoxy-6-[(4-fluorophenyl)-sulfonyl]-amino-α-L-sorbofuranoside (Compound No. 40);
1-O-Dodecyl-2,3-O-isopropylidene-6-deoxy-6-(ethylsulfonyl)-amino-α-L-sorbofuranoside (Compound No 41);
1-O-Dodecyl-2,3-O-isopropylidene-6-deoxy-6-{(4-fluoro-phenyl)-carbonyl}-amino-α-L-sorbofuranoside (Compound No. 42);
1-O-Dodecyl-2,3-O-isopropylidene-6-deoxy-6-{[3-(1,3-benzodioxol-5-yl)-propanoyl]-amino}-α-L-sorbofuranoside (Compound No. 43);
1-O-Dodecyl-2,3-O-isopropylidene-6-deoxy-6-{[{(4-[2-hydroxy-2-oxo-ethyl]-phenyl)-amino}-carbonyl]-amino}-α-L-sorbofuranoside (Compound No. 44);
1-O-Dodecyl-2,3-O-isopropylidene-6-deoxy-6-{[(butyl-amino)-carbonyl]-amino}-α-L-sorbofuranoside (Compound No. 45);
1-O-Dodecyl-2,3-O-isopropylidene-6-deoxy-6-({[(4-fluoro-phenyl)-amino]-thiocarbonyl}-amino)-α-L-sorbofuranoside (Compound No. 46);
Hydrochloride salt of 1-O—)-Dodecyl-2,3-O-isopropylidene-6-deoxy-6-{1-[4-({4-ethoxy-3-[5-{1-methyl-3-propyl-7-oxo-1,6-dihydro-pyrazolo[4,3-d]-pyrimidinyl}]-phenyl}-sulfonyl)-piperazinyl]}-α-L-sorbofuranoside (Compound No. 47)
1-O-[6-{(4-Nitro-phenyl)-amino-carbonyloxy}-hexyl]-2,3;4,6-di-O-isopropylidene-α-L-sorbofuranoside (Compound No. 48);
1-O-[6-{(4-Chloro-phenyl)-amino-carbonyloxy}-hexyl]-2,3;4,6-di-O-isopropylidene-α-L-sorbofuranoside (Compound No. 49);
1-O-[6-{(4-Methoxyl phenylamino-carbonyloxy)-hexyl]-2,3;4,6-di-O-isopropylidene-α-L-sorbofuranoside (Compound No. 50);
1-O-{6-[(4-Methyl-phenyl)-amino-carbonyloxy}-hexyl]-2,3;4,6-di-O-isopropylidene-α-L-sorbofuranoside (Compound No. 51);
(4ξ)-1-O-Dodecyl-2,3-O-isopropylidene-4-C-methyl-6-deoxy-α-L-erythro-hex-2-ulofuranoside (Compound No. 52);
(4ξ)-1-O-Dodecyl-2,3-O-isopropylidene-4-C-methyl-4-O-{[(phenylsulfonyl)-amino]-carbonyl}-6-deoxy-α-L-erythro-hex-2-ulofuranoside (Compound No. 53);
(4ξ)-1-O-Dodecyl-2,3-O-isopropylidene-4-O-[{[(4-methyl-phenyl)-sulfonyl]-amino}-carbonyl]-6-deoxy-α-L-erythro-hex-2-ulofuranoside (Compound No. 54);
(4ξ)-1-O-Dodecyl-2,3-O-isopropylidene-4-C-methyl-4-O-[{[(4-chloro-phenyl)-sulfonyl]-amino}-carbonyl]-6-deoxy-α-L-erythro-hex-2-ulofuranoside (Compound No. 55);
(4ξ)-1-Dodecyl-2,3-O-isopropylidene-4-C-methyl-4-O-{[{2,5-dichloro-phenyl-sulfonyl}-amino]-carbonyl}-6-deoxy-α-L-erythro-hex-2-ulofuranoside (Compound No. 56);
(4ξ)-1-O-Dodecyl-2,3-O-isopropylidene-4-C-methyl-4-O-[{[(2-methyl-phenyl)-sulfonyl]-amino}-carbonyl]-6-deoxy-α-L-erythro-hex-2-ulofuranoside (Compound No. 57);
(4ξ)-1-O-Dodecyl-2,3-O-isopropylidene-4-C-heptyl-4-O-[2-(1-piperidinyl)-ethyl]-6-deoxy-α-L-erythro-hex-2-ulofuranoside (Compound No. 58);
(4ξ)-1-O-Dodecyl-2,3-O-isopropylidene-4-C-heptyl-4-O-[2-(1-azepanyl)-ethyl]-6-deoxy-α-L-erythro-hex-2-ulofuranoside (Compound No. 59);
(4ξ)-1-O-Dodecyl-2,3-O-isopropylidene-4-C-heptyl-4-O-[2-(1-morpholinyl)-ethyl]-6-deoxy-α-L-erythro-hex-2-ulofuranoside (Compound No. 60);
(4ξ)-1-O-Dodecyl-2,3-O-isopropylidene-4-C-heptyl-4-O-[2-(1-pyrrolidinyl)-ethyl]-6-deoxy-α-L-erythro-hex-2-ulofuranoside (Compound No. 61);
(4ξ)-1-O-Dodecyl-2,3-O-isopropylidene-4-C-heptyl-4-O-heptyl-6-deoxy-α-L-erythro-hex-2-ulofuranoside (Compound No. 62);
(4ξ)-1-O-Dodecyl-2,3-O-isopropylidene-4-C-methyl-4-O-[2-(1-dimethylamino)-propyl]-6-deoxy-α-L-erythro-hex-2-ulofuranoside (Compound No. 63);
(4ξ)-1-O-Dodecyl-2,3-O-isopropylidene-4-C-methyl-4-O-[{[(2-azepanyl)-sulfonyl]-amino}-carbonyl]-6-deoxy-α-L-erythro-hex-2-ulofuranoside (Compound No. 64);
1-O-Heptyl-2,3-O-isopropylidene-6-O-{[(4-methyl-phenyl)-amino]-carbonyl}-α-L-sorbofuranoside (Compound No. 65),
1-O-Dodecyl-2,3-O-isopropylidene-6-deoxy-6-[({[2-(carboxymethyl)phenyl]amino}-carbonyl)amino]-α-L-sorbofuranoside (Compound No. 66);
1-O-Dodecyl-2,3-O-isopropylidene-6-deoxy-6-[({[3-(carboxymethyl)phenyl]amino}-carbonyl)amino]-α-L-sorbofuranoside (Compound No. 67);
1-O-Dodecyl-2,3-O-isopropylidene-6-deoxy-6-{[(4-trifluoromethyl)benzoyl]amino}-α-L-sorbofuranoside (Compound No. 68);
1-O-Dodecyl-2,3-O-isopropylidene-6-deoxy-6-{[(4-fluorophenyl)acetyl}amino]-α-L-sorbofuranoside (Compound No. 69);
1-O-Dodecyl-2,3-O-isopropylidene-6-deoxy-6-{(3-fluorobenzoyl) amino}-α-L-sorbofuranoside (Compound No. 70);
1-O-Dodecyl-2,3-O-isopropylidene-6-deoxy-6-{(quinolin-2-ylcarbonyl)amino}-α-L-sorbofuranoside (Compound No. 71);
1-O-Dodecyl-2,3-O-isopropylidene-6-deoxy-6-{(2-thienylacetyl)amino}-α-L-sorbofuranoside (Compound No. 72);
1-O-Dodecyl-2,3-O-isopropylidene-6-deoxy-6-{[(4-methoxyphenyl)acetyl]amino}-α-1-sorbofuranoside (Compound No. 73);
1-O-Dodecyl-2,3-O-isopropylidene-6-deoxy-6-{[(3-fluorophenyl)acetyl]amino}-α-L-sorbofuranoside (Compound No. 74);
1-O-Dodecyl-2,3-O-isopropylidene-6-deoxy-6-{(3,4-dimethoxybenzoyl)amino}-α-L-sorbofuranoside (Compound No. 75);
1-O-Dodecyl-2,3-O-isopropylidene-6-deoxy-6-{(3,6-isoquinolin-1-ylcarbonyl)amino}-α-L-sorbofuranoside (Compound No. 76);
1-O-Dodecyl-2,3-O-isopropylidene-6-deoxy-6-{[4-(acetylamino)benzoyl]amino}-α-L-sorbofuranoside (Compound No. 77);
1-O-Dodecyl-2,3-O-isopropylidene-6-deoxy-6-{[(pyridin-4-yl)-carbonyl]-amino}-α-L-sorbofuranoside (Compound No. 78);
1-O-Dodecyl-2,3-O-isopropylidene-6-deoxy-6-{[(2,6-dichloropyridin-4-yl)-carbonyl]-amino}-α-L-sorbofuranoside (Compound No. 79);
1-O-Dodecyl-2,3-O-isopropylidene-6-deoxy-6-{[(quinolin-3yl)-carbonyl]-amino}-α-L-sorbofuranoside (Compound No. 80);
1-O-Dodecyl-2,3-O-isopropylidene-6-deoxy-6-{[(5-methyl-3-phenylisoxazol-4-yl)-carbonyl]-amino}-α-L-sorbofuranoside (Compound No. 81);
1-O-Dodecyl-2,3-O-isopropylidene-6-deoxy-6-{(phenyl)acetyl}-amino-α-L-sorbofuranoside (Compound No. 82);
1-Dodecyl-2,3-O-isopropylidene-6-deoxy-6{[(4-chlorophenyl)acetyl]amino-α-L -sorbofuranoside (Compound No. 83);
1-O-Dodecyl-2,3-O-isopropylidene-6-deoxy-6-{[(3,5-difluorophenyl)acetyl]amino}-α-L-sorbofuranoside (Compound No. 84);
1-O-Dodecyl-2,3-O-isopropylidene-6-deoxy-6-{[(3-methoxyphenyl)acetyl]amino}-α-L-sorbofuranoside (Compound No. 85);
1-O-Dodecyl-2,3-O-isopropylidene-6-deoxy-6-{[(3-chlorophenyl)acetyl]amino}-α-L-sorbofuranoside (Compound No. 86);
1-O-Dodecyl-2,3-O-isopropylidene-6-deoxy-6-{[(2-methoxyphenyl)acetyl]amino}-α-L-sorbofuranoside (Compound No. 87);
1-O-Dodecyl-2,3-O-isopropylidene-6-deoxy-6-{[(2,4-difluorophenyl)acetyl]amino}-α-L-sorbofuranoside (Compound No. 88);
1-O-Dodecyl-2,3-O-isopropylidene-6-deoxy-6-{[(2,6-chlorophenyl)acetyl]amino}-α-L-sorbofuranoside (Compound No. 89);
1-O-Dodecyl-2,3-O-isopropylidene-6-deoxy-6-{[(4-methylphenyl)acetyl]amino}-α-L-sorbofuranoside (Compound No. 90);
1-O-Dodecyl-2,3-O-isopropylidene-6-deoxy-6-{[(3,4-difluorophenyl)acetyl]amino}-α-L-sorbofuranoside (Compound No. 91);
1-O-Dodecyl-2,3-O-isopropylidene-6-deoxy-6-{[(2,5-difluorophenyl)acetyl]amino}-α-L-sorbofuranoside (Compound No. 92);
1-O-Dodecyl-2,3-O-isopropylidene-6-deoxy-6-{[(2,4,5-trifluorophenyl)acetyl]amino}-α-L-sorbofuranoside (Compound No. 93);
1-O-Dodecyl-2,3-O-isopropylidene-6-deoxy-6-{[(3,4-dichlorophenyl)acetyl]amino}-α-L-sorbofuranoside (Compound No. 94);
1-O-Dodecyl-2,3-O-isopropylidene-6-deoxy-6-{[(4-hydroxyphenyl)acetyl]amino}-α-L-sorbofuranoside (Compound No. 95);
1-O-Dodecyl-2,3-O-isopropylidene-6-deoxy-6-{[(2-methylphenyl)acetyl]amino}-α-L-sorbofuranoside (Compound No. 96);
1-O-Dodecyl-2,3-isopropylidene-6-deoxy-6-{[(2-chlorophenyl)acetyl]amino}-α-L-sorbofuranoside (Compound No. 97);
1-O-Dodecyl-2,3-O-isopropylidene-6-deoxy-6-[(1,3-benzodioxol-5-ylacetyl)amino]-L-sorbofuranoside (Compound No. 98);
1-O-Dodecyl-2,3-O-isopropylidene-6-deoxy-6-{[3-hydroxyphenyl)acetyl]amino}-α-L-sorbofuranoside (Compound No. 99);
1-Dodecyl-2,3-O-isopropylidene-6-deoxy-6-{[(4-hydroxy-3-fluorophenyl)acetyl]amino}-α-L-sorbofuranoside (Compound No. 100);
1-O-Dodecyl-2,3-O-isopropylidene-6-deoxy-6{[(4-isopropylphenyl)acetyl]amino}-α-L-sorbofuranoside (Compound No. 101);
1-O-Dodecyl-2,3-O-isopropylidene-6-deoxy-6-{[biphenyl-4-ylacetyl]amino}-α-L-sorbofuranoside (Compound No. 102);
1-O-Dodecyl-2,3-O-isopropylidene-6-deoxy-6-{[(3-methylphenyl)acetyl]amino}-α-L-sorbofuranoside (Compound No. 103);
1-O-Dodecyl-2,3-O-isopropylidene-6-deoxy-6-{[(2-fluoro-6-chlorophenyl)acetyl]amino}-α-L-sorbofuranoside (Compound No. 104);
1-O-Dodecyl-2,3-O-isopropylidene-6-deoxy-6-{[(2-chloro-4-fluorophenyl)acetyl]amino}-α-L-sorbofuranoside (Compound No. 105);
1-O-Dodecyl-2,3-O-isopropylidene-6-deoxy-6{[(4-trifluoromethoxyphenyl)acetyl]amino}α-L-sorbofuranoside (Compound No. 106);
1-O-Dodecyl-2,3-O-isopropylidene-6-deoxy-6-{[(2-trifluoromethoxyphenyl)acetyl]amino}-α-L-sorbofuranoside (Compound No. 107);
1-O-Dodecyl-2,3-O-isopropylidene-4-O-ethyl-6-deoxy-6-{[(4-fluorophenyl)acetyl]amino}-α-L-sorbofuranoside (Compound No. 108);
1-O-(2-butoxyethyl)-2,3-O-isopropylidene-6-deoxy-6-{[(3,4-difluorophenyl)-acetyl]amino}-α-L-sorbofuranose (Compound No. 109);
1-O-(2-butoxyethyl)-2,3-O-isopropylidene-6-deoxy-6-{[(3,4-dichlorophenyl)acetyl]amino}-α-L-sorbofuranose (Compound No. 110);
1-O-(2-butoxyethyl)-2,3-O-isopropylidene-6-deoxy-6-{[(4-methoxyphenyl)acetyl]amino}α-L-sorbofuranose (Compound No. 111);
1-O-(2-butoxyethyl-2,3-isopropylidene-6-deoxy-6-{[(3-methoxyphenyl)acetyl]amino}-α-L-sorbofuranose (Compound No. 112);
1-O-(2-butoxyethyl)-2,3-O-isopropylidene-6-deoxy-6-{[(4-fluorophenyl)acetyl]amino}-α-L-sorbofuranoside (Compound No. 113);
(4ε)-1-O-Heptyl-2,3-O-isopropylidene-4-C-methyl-4-O-[{[(4-methylphenyl)sulphonyl]amino}-carbonyl]-6-deoxy-α-L-erythro-hex-2-ulofuranoside (Compound. No. 114);
(4ε)-1-O-Heptyl-2,3-O-isopropylidene-4-C-methyl-4-O-[{[(2-methylphenyl)sulphonyl]amino}-carbonyl]-6-deoxy-α-L-erythro-hex-2-ulofuranoside (Compound No. 115);
(4ε)-1-O-Heptyl-2,3-O-isopropylidene-4-C-methyl-4-O-[{[(4-chlorophenyl)sulphonyl]amino}-carbonyl]-6-deoxy-α-L-erythro-hex-2-ulofuranoside (Compound No. 116);
(4ε)-1-O-Heptyl-2,3-O-isopropylidene-4-C-methyl-4-O-[{[phenylsulphonyl]amino}carbonyl]-6-deoxy-α-L-erythro-hex-2-ulofuranoside (Compound No. 117); or
(4ε)-1-O-Heptyl-2,3-O-isopropylidene-4-C-methyl-4-O-[{[(4-fluorophenyl)sulphonyl]amino}-carbonyl]-6-deoxy-α-L-erythro-hex-2-ulofuranoside (Compound No. 118).

12. A pharmaceutical composition comprising a compound of claim 1 and at least one pharmaceutically acceptable excipient.

13. A method of inhibiting or preventing auto immune disease, comprising administering a therapeutically effective amount of the composition of claim 12 to a patient in need thereof.

14. A method of treating bronchial asthma comprising administering a therapeutically effective amount of the composition of claim 12 to a patient in need thereof.

15. A method of treating chronic obstructive pulmonary disorder, comprising administering the pharmaceutical composition of claim 12 to a patient in need thereof.

16. A method of treating rheumatoid arthritis, comprising administering a therapeutically effective amount of the composition of claim 12 to a patient in need thereof.

17. A method of treating Type I diabetes, comprising administering a therapeutically effective amount of the composition of claim 12 to a patient in need thereof.

18. A method of treating multiple sclerosis, comprising administering a therapeutically effective amount of the composition of claim 12 to a patient in need thereof.

19. A method of treating allograft rejection, comprising administering a therapeutically effective amount of the composition of claim 12 to a patient in need thereof.

20. A method of treating psoriasis, comprising administering a therapeutically effective amount of the composition of claim 12 to a patient in need thereof.

21. A method of treating inflammatory bowel disease, comprising administering the pharmaceutical composition of claim 12 to a patient in need thereof.

22. A method of treating ulcerative colitis, comprising administering the pharmaceutical composition of claim 12 to a patient in need thereof.
